(19) United States
(12) Patent Application Publication
(10) Pub. No.: US 2012/0202985 A1
(43) Pub. Date: Aug. 9, 2012

(54) Hyper IgE Animal Model with Enhanced Immunoglobulin Heavy Chain Class Switching to C-epsilon

(75) Inventors: Shahram Misaghi, Clayton, CA (US); Ali Zarrin, Brisbane, CA (US)

(73) Assignee: Genentech Inc., South San Francisco, CA (US)

(21) Appl. No.: 13/255,226

(22) PCT Filed: Feb. 26, 2010
(86) PCT No.: PCT/US2010/025507

§ 371 Date: Apr. 25, 2012

Related U.S. Application Data
(60) Provisional application No. 61/156,299, filed on Feb. 27, 2009.

(51) Int. Cl. A01K 67/027; G01N 33/566; C08B 37/00; C12N 5/10; C12N 15/85; G01N 21/64

(52) U.S. Cl. 536/123.1; 435/320.1; 435/455; 800/9; 435/7.21; 435/325; 800/25; 435/7.92

(57) ABSTRACT

Described herein are recombinant non-human cells and animals having an alteration of the Sε region such that there is an elevated IgE level. Also described herein is an alteration in the IgH locus allows for enhanced class switch recombination (CSR) such that the desired heavy chain isotype is expressed at an elevated level relative to an unmodified cell.

### CROSS-REFERENCE TO RELATED APPLICATIONS

This application is made under 35 U.S.C. §371 based on International Application PCT/US2010/025507 filed on Feb. 26, 2010 and claims the benefit of priority under 35 U.S.C. §119(e) to U.S. Provisional Ser. No. 61/156,299 filed on Feb. 27, 2009, all of which are hereby incorporated in their entirety by reference.

### SEQUENCE LISTING

A sequence listing comprising SEQ ID NOS: 1-20 is attached hereto as Table 1. Each sequence provided in the sequence listing is incorporated herein by reference, in its entirety, for all purposes.

### TECHNICAL FIELD

This disclosure relates to a recombinant mouse and methods for testing allergy treatments.

### BACKGROUND

Asthma is a debilitating disease affecting one fifth of the population of the developed world. Severe asthma is a major cause of hospitalization and health care costs. In clinical practice, asthma is classified as atopic or nonatopic, according to the presence or absence of circulating IgE directed against local aeroallergens detected by skin prick test (SPT) or in vitro techniques (RAST or ELISA). These IgE antibodies interact with the high-affinity IgE receptor (FceRI) on mast cells, which may result in immediate hypersensitivity on allergen provocation and acute exacerbation of disease.

About one third of adult patients with asthma are classified as nonatopic. They tend to have more severe disease, often associated with chronic rhinosinusitis, but apart from their lack of acute reactivity to allergens, their disease is clinically similar.

An allergy is an immunological reaction, generally of the immediate hypersensitivity type, to a particular type of antigen termed an allergen. Such reactions underlie attacks of anaphylaxis, allergic rhinitis (hay fever), hives, and allergic asthma, and may be triggered by common allergens such as ragweed, pollen, bee or wasp venom, animal dander, mold, or a component of house dust (such as mites).

There is a close concordance between asthma, allergic rhinitis and atopic dermatitis; the presence of one of these entities increases the relative risk of the other two by 3- to 30-fold over the lifetime of the subject. All three of these diseases are associated with high levels of nonspecific and antigen-specific serum immunoglobulin E (IgE).

In humans, immediate hypersensitivity (1H) is mediated by antibodies of the IgE isotype anchored to the surfaces of mast cells and basophils in the skin and elsewhere. Binding of antigen to these cell-bound IgE molecules triggers release of mediators such as histamine from the cells, which mediators induce the clinical phenomena such as tissue swelling, itching, or bronchial smooth muscle contraction that typify an allergic reaction.

IgE antibodies specific for a given allergen are produced and secreted by B lymphocytes upon contact with that allergen. Initially, B lymphocytes (or B cells) express antibodies of the IgM isotype, with each B cell committed to producing antibody specific for a particular antigenic determinant. Contact with both an allergen bearing that antigenic determinant, and certain factors produced by T lymphocytes, will induce the B cell to undergo what is termed an antibody heavy chain class switch, in which the antigen-specific portion of the antibody produced by the B cell remains the same, but it is attached to the ε-heavy chain (to yield IgE antibody) rather than the μ-heavy chain of the IgM isotype. Such a class switch is apparently permanent for a given B cell, which thereafter secretes IgE antibody specific for the allergen whenever stimulated to do so.

Ovalbumin (OVA)-induced asthma in mice is one of the most commonly used models of human asthma. Th2 type cells are believed to be critical in pathogenesis of OVA-induced asthma. While we know that Th2 lymphocytes play an important role in the initiation, progression and persistence of allergic asthma, there is a lot to be understood about the immunoregulatory mechanisms. This model fails to mimic human disease associated with hyper-IgE.

Allergic asthma models have also been described in large animal models, e.g., cats, dogs, pigs, sheep, and monkeys. Among these species, the feline one is of particular interest because cats spontaneously develop idiopathic asthma. However, large animal models are expensive and time consuming and have limited availability of immunological and/or molecular tools.

US 6118044 (2000) provides transgenic mice which constitutively express an antibody-type molecule encoded by a transgene and which has an IgE heavy chain constant region and is specific for a pre-defined antigen (i.e., TNP). It does not provide a polyclonal response to an unknown or non-specific antigen.

Currently there is no good model of chronic airway disease as they lack many key pathological features of human asthma such as mast cell infiltration of smooth muscle. In addition, almost all of them resolve spontaneously over time because mice don't get asthma. Thus, it would be desirable to have a non-human animal model that will allow the generation of an elevated IgE response relative to a native non-human animal wherein the antibody repertoire is polyclonal.

Currently, common treatments for allergy include avoidance of the suspected allergen; injections of the allergen as immunotherapy to stimulate certain protective mechanisms and thereby eventually desensitize the host to the allergen; drugs such as corticosteroids, which interfere with the release of the mediators of allergy from mast cells; and drugs such as antihistamines, which block the biological action of the released mediators. However, there is no good animal model for testing allergy therapies, especially therapeutic agents that will block IgE isotype switching in B cells without adverse effects.

Thus, it is desirable to have cells and/or animals that have the ability to generate an elevated IgE response. In other words, selectively enhancing the rate of chain switch recombination for IgE production would be desirable.

### BRIEF SUMMARY OF THE INVENTION

Despite ongoing research, there still remains a need for an in vivo model for IgE involvement in asthma, allergies and other immunologic pathologies that provides a polyclonal response to a non-specific antigen, i.e., an antigen that is not predefined. Also lacking is an in vivo animal model for hyper-IgE generation wherein there is an elevated serum IgE response.

Provided herein is a recombinant non-human animal, and a method for using it, that is useful as a reliable model for the search for, and/or evaluation of, anti-allergic drugs.

An animal model that has a genomic structure within the immunoglobulin locus that is substantially similar to the wild-type (i.e., native or unmodified) immunoglobulin locus and retains the potential to provide a full repertoire of immunoglobulins in response to antigen challenge would allow the search for, and/or evaluation of, anti-allergic drugs that inhibit IgE isotype switching in B cells.

The present disclosure provides an animal model for testing allergy therapies. The animal model provides a wide diversity of antibody production in response to antigenic challenge, producing the full diversity of antibody isotypes and full complement of specificities to epitopes on the antigen. The animal model further provides a means to further understand the physiological importance of IgE to allergy and asthma.

In a first embodiment there is provided a targeting vector comprising:
a) a fragment of DNA homologous to the 5′ end of the switch region to be altered (the 5′ arm/acceptor) is selected from the group consisting of at least 1500 nucleotides, at least 1800 nucleotides, at least 2000 nucleotides, at least 2200 nucleotides and at least 2400 nucleotides corresponding to Nucleotides 25470628 to 25468161 of NCBI accession number NT_{—}166318 (Mus musculus chromosome 12 genomic contig, strain C57BU6J) (SEQ ID NO:5);
b) a selectable gene marker;
c) a desired/donor DNA sequence encoding a donor switch region; and
d) a second fragment of DNA homologous to the 3′ end of the switch region to be altered (the 3′ arm/acceptor) is selected from the group consisting of at least 1500 nucleotides, at least 1800 nucleotides, at least 2000 nucleotides, at least 2200 nucleotides, at least 2400 nucleotides and at least 2800 nucleotides corresponding to Nucleotides 25470628 to 2546816 of NCBI accession number NT_{—}166318 (Mus musculus chromosome 12 genomic contig, strain C57BU6J 1) (SEQ ID NO:8).

In one aspect the targeting vector has a 5′ arm comprising SEQ ID NO:4 or 5. In an embodiment the 5′ arm comprises residues 25-2471, inclusive, of SEQ ID NO:4. In a further aspect, the 5′ arm is homologous to a region 3′ of the endogenous Iε and 5′ of the endogenous SE. In a second aspect, the targeting vector has a 3′ arm comprising SEQ ID NO:7 or 8. In an embodiment the 3′ arm comprises residues 2-2495, inclusive, of SEQ ID NO:7. In a third aspect, the targeting vector has a selectable gene marker that is selected from the group consisting of Neomycin and tymidine kinase. In a further aspect, the selectable gene marker is Neomycin. In a fourth aspect, the targeting vector has the selectable gene marker flanked by loxp sites. In a fifth aspect, the targeting vector has a desired switch region that is selected from the group consisting of human and mouse. In a sixth aspect, the desired switch region is selected from Sμ, Sγ1, Sγ2a, Sγ2b and Sγ3. In a seventh aspect, the desired switch region is the HindIII/NheI fragment containing most of mouse Sμ region. In an eighth aspect, the desired switch region comprises Nucleotides 25617172 to 25615761 of NCBI accession number NT_{—}166318 (Mus musculus chromosome 12 genomic contig, strain C57BU6J) (SEQ ID NO:6). In a ninth aspect the Sμ region comprises a 4.9 kb NheI-HindIII fragment was subcloned from a plasmid containing a genomic fragment isolated from BAC clone RP23-354L16.

In second embodiment there is provided a method for producing an altered embryonic stem cell in vitro, comprising the steps of:
a) Altering the genomic DNA in said cell to enhance the probability of class switch recombination (CSR) to express the Cε selected from
b) increasing the Sε length by adding at least one additional Sε copy in tandem with the endogenous Sε region;
c) Sε region substitution; and
d) Selecting the cell for correctly altered genomic DNA.

In one aspect, the alteration is a substitution of a switch region selected from Sμ, Sγ1, Sγ2a, Sγ2b and Sγ3 for the Sε region. In a further aspect, the alteration is a substitution of a Sμ region for the Sε region. In a further aspect, the alteration is a substitution of any acceptor S region (Sγ1, Sγ2a, Sγ2b and Sγ3, Sa) with Sm or vice versa.

In a third embodiment there is provided a method for producing an altered embryonic stem cell (ESC) in vitro, comprising the steps of:
a) Using the vector according to claim 1 to exchange the Sμ for the Sε region
b) Selecting the cell for correctly altered genomic DNA.

In one aspect, the method provides an alteration that is a substitution of a switch region selected from Sμ, Sγ1, Sγ2a, Sγ2b and Sγ3 for the Sε region. In a further aspect, the method provides an alteration that is a substitution of a Sμ region for the Sε region. In another aspect, the method provides that the ESC are from a mouse strain selected from BALB/c or C57BU6.

In a fourth embodiment there is provided a non-human animal wherein:
a) At least one allele of the IgH locus has been altered to enhance the rate of IgE expression/production/secretion/relative to a non-altered allele; and
b) Has an IgE profile selected from the group consisting of:
   i. The IgE fraction of all serum antibodies is greater than 0.04%;
   ii. The IgE serum concentration is above 4,000 ng/ml;
   iii. The IgG/IgE ratio is less than 10.

In a first aspect, the non-human mammal has an IgE serum level greater than 4,000 μg/ml, greater than 10,000 μg/ml, greater than 15,000 μg/ml, greater than 30,000 μg/ml, greater than 90,000 μg/ml, greater than 10 μg/ml, greater than 20 μg/ml, greater than 30 μg/ml, greater than 40 μg/ml, greater than 50 μg/ml, greater than 60 μg/ml, greater than 70 μg/ml, greater than 80 μg/ml, greater than 90 μg/ml or greater than 100 μg/ml. In a second aspect, the non-human mammal has an IgG/IgE ratio that is between 0.1 and 10. In a third aspect, the non-human mammal having an unchallenged (i.e., resting) IgE serum concentration of between 100 ng/mL and 10000 ng/mL. In a fourth aspect, the non-human mammal has a challenged (i.e., activated or stimulated) IgE serum concentration of between 1000 ng/mL and 1000000 ng/mL. In a fifth aspect, the animal model is a nonhuman vertebrate. In a sixth aspect, the animal model is a mouse, rat, guinea pig, rabbit, or primate. In a seventh aspect, the genome of the non-animal described herein has had the Sε region of the IgH locus altered to express/produce more IgE. In an eighth aspect, the non-human animal/mammal model has an alteration that is achieved by gene targeting. In a ninth aspect, the non-human mammal has an IgE fraction of at least 0.04%, 0.1%, 0.2%, 0.3%, 0.4%, 0.5%, 0.6%, 0.7%, 0.8%, 0.9% or 1.0%.

In a fifth embodiment there is provided a method of testing an allergy therapy using the animal model comprising exposing said animal to an allergen prior to, simultaneous with or after the administration of said method of treatment for allergic disorders and evaluating the IgE response. In a first aspect of the method the IgE levels in response to antigen challenge is less than without the allergy therapy. In a second aspect, the test animal and the control animal are littermates.

In a sixth embodiment there is provided a use of a compound identified by the method of testing an allergy therapy as a medicament for the treatment of an allergy.

In a seventh embodiment there is provided a cell line obtainable from the animal model described herein.

In a eighth embodiment there is provided a cell isolated from an animal model described herein.

In a ninth embodiment there is provided a process for making a non-human animal model, said process comprising:
a) microinjecting linearized fragments of plasmids encoding SEQ ID NO:6 (Sμ) into a fertilized egg of a mouse such that the fragment is incorporated in the genomic DNA upstream from and operably linked to the Cε-encoding region,
b) transferring said fertilized egg to the oviduct of a female mouse which has previously been treated to induce pseudopregnancy, and c) allowing said egg to develop in the uterus of the female mouse.

In a tenth embodiment there is provided a recombinant mouse comprising, in its germline, a modified genome wherein said modification comprises at least one allele of the IgH locus altered to enhance the rate of IgE production. In a first aspect, the recombinant mouse has an alteration that comprises replacing the Sε with the Sμ region or a functional portion thereof. In a second aspect, the Sμ functional portion is between at least 1 kb and 10 kb in length.

Other objects, features and advantages of the present invention will become apparent from the following detailed description. It should be understood, however, that the detailed description and specific examples, while indicating preferred embodiments of the invention, are given by way of illustration only, since various changes and modifications within the scope and spirit of the invention will become apparent to one skilled in the art from this detailed description.

### BRIEF DESCRIPTION OF THE DRAWINGS

FIG. 1 is a schematic of the genetic alterations of the mouse IgH locus. (A) Genomic organization of the variable region up to Cm in germ line configuration. (B) V(D)J recombination assembles the functional coding variable region generating a large pool of low affinity IgM producing B cells. (C) Activation of B cells accompanied by induction of AID and germline transcription results in SHM, where point mutations are introduced into assembled V region (asterisks). AID-mediated DSBs (lightning symbol) in Sm and a downstream S region (e.g., Sg1) are joined to generate new isotypes (e.g., IgG1) transcript. In addition, an excised circular fragment is generated by joining the intervening sequence.

FIG. 2A illustrates a schematic of the gene targeting strategy and recombination sites for modification of the mouse Sε region. The structure of the targeted allele after Cre_loxP recombination is illustrated at the bottom. Restriction enzyme cleavage sites are designated. R1 indicates the splice site for EcoRI. All other restriction enzymes have their full name.

FIG. 2B is a schematic of the targeting vector, pSW312. See Example 3.

FIG. 3 is a schematic of the overall mouse IgH locus before and after replacement of the Sε region with a donor switch region. In this diagram the donor switch region is Sμ. In the upper panel is the unmodified IgH locus; the lower panel illustrates a modified IgE locus as described herein.

FIG. 4A is a schematic of the unmodified (i.e., wild-type) genomic locus and illustrates the relative locations of the restriction sites, probe and switch region. The 5′ homology arm is represented by the black box. The 3′ homology arm is represented by the gray box.

FIG. 4B is a schematic of the modified genomic locus and illustrates the relative locations of the restriction sites, probe and switch region with Sε replaced with Sm. The 5′ homology arm is represented by the black box. The 3′ homology arm is represented by the gray box.

FIG. 4C is a Southern blot confirming the replacement of the Sε with Sp. While wild-type B6 samples show only one band of relevant size indicating existence of a single genomic Is region, targeted embryonic stem cell samples show the wild-type and the targeted Sε sites (where Sε is replaced with Sμ) manifested as two bands with distinct size differences. This shows successful targeting and replacement of the intended switch region.

FIG. 4D s a Southern blot confirming the replacement of the Sε with Sμ. While wild-type B6 samples show only one band of relevant size indicating existence of a single genomic Iε region, targeted embryonic stem cell samples show the wild-type and the targeted Sε sites (where Sε is replaced with Sμ) manifested as two bands with distinct size differences. This shows successful targeting and replacement of the intended switch region.

FIGS. 5-11 show nucleotides described herein. The nucleotide base codes are: A or a is adenine; C or c is cytosine; G or g is guanine; T or t is thymine; M or m is adenine or cytosine; S or s is cytosine or guanine; and N or n is adenine or cytosine or guanine or thymine.

FIG. 5 shows two nucleotide sequences from the Mouse. The motif GGGCTGGGCTG (SEQ ID NO:1 shown in FIG. 5A) is found in Sm and Sε and a second motif GAGCTGACT is slightly modified in the Sε region as GAGCTGAGCT (has an added G relative to the Sm motif) (SEQ ID NO:2 shown in FIG. 5B).

FIG. 6 shows a 2055 bp (SEQ ID NO:3) deleted from the BamHI/PVuI fragment of the IgH locus.

FIG. 7 shows A) SEQ ID NO:4, the 2471 bp 5′arm (for 129 mice) and B) SEQ ID NO:5, the 2467 bp 5′ arm (for C57BI/6J strain) corresponding to Nucleotides 25470628 to 25468161 of NCBI NT_{—}166318.

FIG. 8 shows SEQ ID NO:6 corresponding to nucleotides 25617172 to 25615761 of NCBI Accession Number NT_{—}166318 (Mus musculus chromosome 12 genomic contig, strain C57BU6J)(in caps) (1141 bp).

FIG. 9 shows A) SEQ ID NO:7, the 3′ arm (129 mouse sequence) and B) SEQ ID NO:8, the 3′ arm (C57BI/6J sequence) corresponding to 25466106 to 25463273 of NCBI NT_{—}166318.

FIG. 10 shows A) the 3.7 kb upstream of BamHI (used to design 5′ probe) (SEQ ID NO:9): and B) the PVUI to ECOR1 fragment (used for 3′ probe design) (SEQ ID NO:10).

FIG. 11 shows probes used in Example 2. A) SEQ ID NO:11, I-mu Forward-1 (21 bp); and B) SEQ ID NO:12, C-epsilon Reverse-1 (30 bp); C) SEQ ID NO:13, E-mu Forward-2 (20 bp); D) SEQ ID NO:14, C-epsilon Reverse-2 (30 bp); E) SEQ ID NO:15, Forward: SM5′ (20 bp); F) SEQ ID NO:16, 9225F (19 bp); G) SEQ ID NO:17, 9518F (26 bp); and H) SEQ ID NO:18, Reverse (30 bp).

FIG. 12 is the retrieved IgE C57BU6 genomic sequence (SE region to be deleted shown in bold, underlined font) from BAC RP23-135L12 (Invitrogen) (SEQ ID NO:19).

FIG. 13 A-D summarizes the FACS data for intracellular levels of various immunoglobulins in wild-type (WT) and heterozygotes (HET) splenocytes following immune stimulation. FIG. 13A is a bar graph showing the IgM levels after lipopolysaccharide (LPS) stimulation is the same for both WT and HET animals. FIG. 13B is a bar graph showing the IgG3 levels after lipopolysaccharide (LPS) stimulation is the same for both WT and HET animals. FIG. 13C is a bar graph showing the IgG1 levels after IL-4 in combination with anti-CD40 (4/40) stimulation is the decreased in HET animals compared to WT. FIG. 13D is a bar graph showing the IgE levels after IL-4 in combination with anti-CD40 (4/40) stimulation is the increased in HET animals compared to WT.

FIG. 14 A-D summarizes the ELISA data for the same splenocytes as used to generate the data presented in FIG. 13. At day 6 post stimulation, supernatants form the same stimulated splenocytes (three Het and three WT mice) that were used for FACS analysis were used in ELISA assay. In agreement to what we observed in FACS analysis, we also observed increase in levels of IgE expression and decrease in levels of IgG1 expression in Het compared to WT when stimulated with IL4/anti-CD40. This suggests that there are more frequent breaks occurring in SmKI site that competes with switching to IgG1 and increases levels of IgE switching. LPS stimulation serves as control and shows that both WT and Het have similar levels of IgM and IgG3, suggesting that the locus is intact and functions normally when other switch sequences are accessible for class switching.

### DETAILED DESCRIPTION

The invention will now be described in detail by way of reference only using the following definitions and examples. All patents and publications, including all sequences disclosed within such patents and publications, referred to herein are expressly incorporated by reference.

Unless defined otherwise herein, all technical and scientific terms used herein have the same meaning as commonly understood by one of ordinary skill in the art to which this invention belongs. Singleton, et al., DICTIONARY OF MICROBIOLOGY AND MOLECULAR BIOLOGY, 2D ED., John Wiley and Sons, New York (1994), and Hale & Marham, THE HARPER COLLINS DICTIONARY OF BIOLOGY, Harper Perennial, NY (1991) provide one of skill with a general dictionary of many of the terms used in this invention. Although any methods and materials similar or equivalent to those described herein can be used in the practice or testing of the present invention, the preferred methods and materials are described. Numeric ranges are inclusive of the numbers defining the range. Unless otherwise indicated, nucleic acids are written left to right in 5′ to 3′ orientation; amino acid sequences are written left to right in amino to carboxy orientation, respectively. Practitioners are particularly directed to Sambrook et al., 1989, and Ausubel F M et al., 1993, for definitions and terms of the art. It is to be understood that this invention is not limited to the particular methodology, protocols, and reagents described, as these may vary.

Numeric ranges are inclusive of the numbers defining the range.

Unless otherwise indicated, nucleic acids are written left to right in 5′ to 3′ orientation; amino acid sequences are written left to right in amino to carboxy orientation, respectively.

The headings provided herein are not limitations of the various aspects or embodiments of the invention which can be had by reference to the specification as a whole. Accordingly, the terms defined immediately below are more fully defined by reference to the specification as a whole.

### DEFINITIONS

Novel recombinant non-human hosts, particularly mammalian hosts, usually murine, are provided, wherein the host is capable of mounting an immune response to an immunogen (also called an antigen). The immune response produced is a full repertoire of antibodies albeit with an elevated IgE component or fraction of the total serum Ig concentration.

By “recombinant” is meant that the DNA of an animal or cell contains a genetically engineered modification. Thus, for example, a “recombinant animal” would be one in which at least a portion of its cells contain a genetic modification as described herein. Similarly, a “recombinant cell” would be one in which its genome has a genetic modification as described herein.

“Non-specific antigen” means any substance (as an immunogen or a hapten) foreign to the body that evokes an immune response either alone or after forming a complex with a larger molecule (as a protein) and that is capable of binding with a product (as an antibody or T cell) of the immune response.

As used herein, “isotype” refers to the antibody class (e.g., IgM or IgG₁) that is encoded by heavy chain constant region genes.

As used herein, “isotype switching” refers to the phenomenon by which the class, or isotype, of an antibody changes from one Ig class to one of the other Ig classes.

As used herein, “nonswitched isotype” refers to the isotypic class of heavy chain that is produced when no isotype switching has taken place; the C_{H} gene encoding the nonswitched isotype is typically the first C_{H} gene immediately downstream from the functionally rearranged VDJ gene.

As used herein, the term “switch sequence” refers to those DNA sequences responsible for switch recombination. During class switch recombination (CSR) a “switch donor” sequence, typically a p switch region, will be 5′ (i.e., upstream) of the region to be deleted during the switch recombination. The “switch acceptor” region will be between the region to be deleted and the replacement constant region (e.g., γ, ε, etc.). As there is no specific site where recombination always occurs, the final gene sequence will typically not be predictable. Switch sequence may be used interchangeably with switch region herein.

In the genetically modified (i.e., recombinant) animal described herein the switch acceptor region is modified to enhance CSR so that the serum IgE levels are elevated.

S regions are large, repetitive intronic sequences that vary greatly in length (repetitive regions range from 2.0 to 6.5 kb in mice). Mammalian S regions are unusually G-rich on the nontemplate strand and are composed primarily of tandem repetitive units within which certain motifs—such as TGGGG, GGGGT, GGGCT, GAGCT, and AGCT predominate.

The term “rearranged” as used herein refers to a configuration of a heavy chain or light chain immunoglobulin locus wherein a V segment is positioned immediately adjacent to a D-J or J segment in a conformation encoding essentially a complete V_{H} or V_{L} domain, respectively. A rearranged immunoglobulin gene locus can be identified by comparison to germline DNA; a rearranged locus will have at least one recombined heptamer/nonamer homology element.

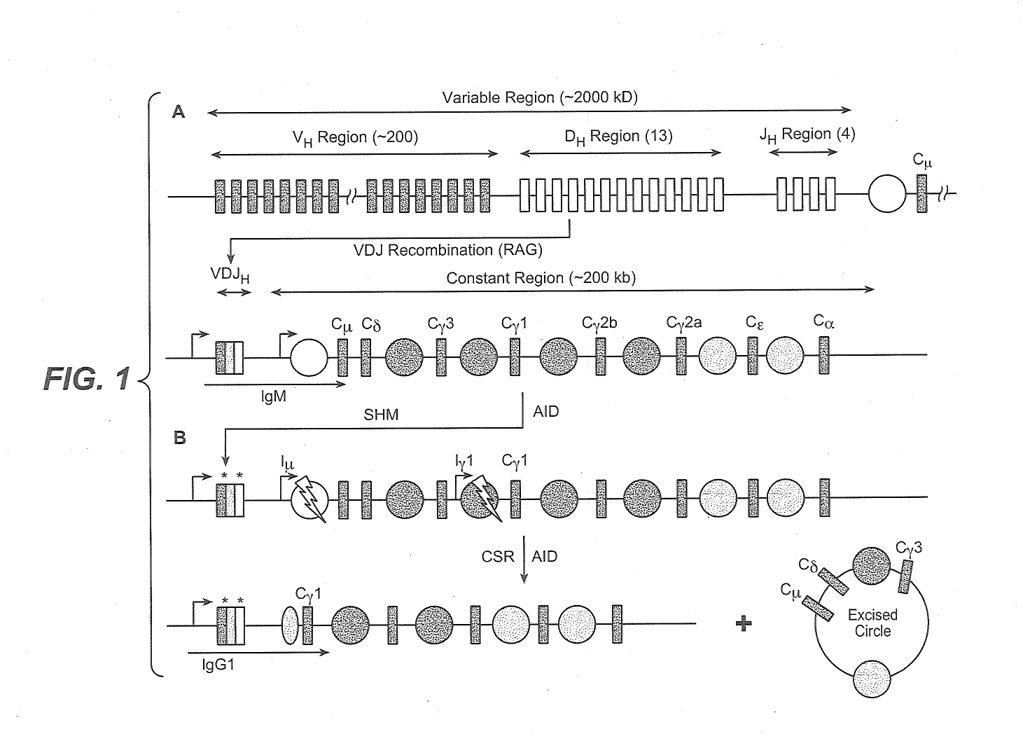

The term “unrearranged” or “germline configuration” as used herein in reference to a V segment refers to the configuration wherein the V segment is not recombined so as to be immediately adjacent to a D or J segment. Reference is made to FIG. 1.

For nucleic acids, the term “substantial homology” indicates that two nucleic acids, or designated sequences thereof, when optimally aligned and compared, are identical, with appropriate nucleotide insertions or deletions, in at least about 80% of the nucleotides, usually at least about 90% to 95%, and more preferably at least about 98 to 99.5% of the nucleotides. Alternatively, substantial homology exists when the segments will hybridize under selective hybridization conditions, to the complement of the strand. The nucleic acids may be present in whale cells, in a cell lysate, or in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCl banding, column chromatography, agarose gel electrophoresis and others well known in the art. See, F. Ausubel, et al., ed. Current Protocols in Molecular Biology, Greene Publishing and Wiley-Interscience, New York (1987).

The nucleic acid compositions of the present invention, while often in a native sequence (except for modified restriction sites and the like), from either cDNA, genomic or mixtures may be mutated, thereof in accordance with standard techniques to provide gene sequences. For coding sequences, these mutations, may affect amino acid sequence as desired. In particular, DNA sequences substantially homologous to or derived from native V, D, J, constant, switches and other such sequences described herein are contemplated (where “derived” indicates that a sequence is identical or modified from another sequence).

A nucleic acid is “operably linked” when it is placed into a functional relationship with another nucleic acid sequence. For instance, a promoter or enhancer is operably linked to a coding sequence if it affects the transcription of the sequence. With respect to transcription regulatory sequences, operably linked means that the DNA sequences being linked are contiguous and, where necessary to join two protein coding regions, contiguous and in reading frame. For switch sequences, operably linked indicates that the sequences are capable of effecting switch recombination.

The design of a non-human animal that responds to foreign antigen stimulation with an antibody repertoire requires that the immunoglobulin genes contained within the animal function correctly throughout the pathway of B-cell development. Correct function of a heavy chain gene includes isotype switching. Accordingly, the genes of the invention are constructed so as to produce isotype switching and one or more of the following: (1) high level and cell-type specific expression, (2) functional gene rearrangement, (3) activation of and response to allelic exclusion, (4) expression of a sufficient primary repertoire, (5) signal transduction, (6) somatic hypermutation, and (7) domination of the IgE antibody locus during the immune response.

In the mouse, CH genes are arranged in the order 5′-V(D)J-Cμ-Cδ-Cγ3-Cγ1-Cγ2b-Cγ2a-Cε-Cα-3′. CSR occurs in switch (S) regions, which are 1- to 10-kilobase (kb) repetitive DNA elements 5′ of individual CH genes. CSR results from recombination between the S region upstream of Cm (Sm) and a downstream S region, accompanied by deletion of intervening sequences.

### Immunoglobulins

The immune system responds to foreign invaders (antigens) by producing antibodies. Antibodies are protein molecules that attach themselves to invading microorganisms and mark them for destruction or prevent them from infecting cells. Antibodies are antigen specific. That is antibodies produced in response to antigen exposure are specific to that antigen.

Mammals produce four isotypes (or classes) of Ig: IgM, IgG, IgE, and IgA, encoded by the μ, γ, ε, and α constant regions, respectively.

Related IgG subclasses are encoded by distinct Cγ regions. Each Ig isotype is specialized for particular modes of antigen removal. IgM, the first isotype synthesized by a B cell, activates complement. IgG, the most abundant isotype in serum, binds receptors on phagocytic cells. IgG antibodies cross the placenta to provide maternal protection to the fetus. IgA antibodies are abundant in secretions, such as tears and saliva; they coat invading pathogens to prevent proliferation. IgE antibodies can provide protection against parasitic nematodes, but in developed countries they are the bad guys: They bind basophils and mast cells, activating histamine release and resulting in an allergic response.

Immunogens (or antigens) can trigger an antibody response. Successful recognition and eradication of many different types of antigens requires diversity among antibodies; their amino acid composition varies allowing them to interact with many different antigens. It has been estimated that humans generate about 10 billion different antibodies, each capable of binding a distinct epitope of an antigen. Although a huge repertoire of different antibodies is generated in a single individual, the number of genes available to make these proteins is limited. Several complex genetic mechanisms have evolved that allow vertebrate B cells to generate a diverse pool of antibodies from a relatively small number of antibody genes.

### B-Cell Development

B cells undergo a series of differentiation checkpoints in the bone marrow and spleen before they become mature functional cells. Decisions as whether to continue differentiation or to undergo cell death occur at these checkpoints and revolve principally around the immunoglobulin B-cell receptor (BCR) and its ability to function as an antigen-binding and signal-transduction molecule. The first two such checkpoints are in the bone marrow at the pro-B to pre-B transition, where the newly synthesized heavy (H) chains associate with surrogate light (L) chains to form a pre-BCR, and at the pre-B to immature B-cell stage, where the H chains associate with conventional L chains to form a BCR. Cells that are unable to form a pre-BCR or BCR undergo apoptosis (programmed cell death), whereas those that can form a BCR continue differentiating. The mature B cell that moves into the periphery can be activated by antigen and become an antibody-secreting plasma cell or memory B cell, which will respond more quickly to a second exposure to antigen. When antigen-activated B cells stop proliferating they can differentiate into mature plasma cells. Plasma cells are essentially ‘antibody factories’. (See Hardy & Hayakawa, B Cell Development Pathways, Annu Rev Immunol. (2001) 19:595-621.)

Initially, all B cells produce IgM antibodies. The V, D and J elements encoding the variable-region domains of the m heavy chain are located adjacent to the Cm exons that encode the IgM C-regions at the 5′ end of the immunoglobulin heavy-chain (IgH) locus. Following appropriate stimulation, B cells can alter the isotype of the antibodies they produce via class switching while retaining their antigenic specificity. Class switching occurs in the heavy chain gene locus by a mechanism called class switch recombination (CSR). This mechanism relies on conserved nucleotide motifs, called switch (S) regions, found in DNA upstream of each constant region gene (except in the 6-chain). In this process, genomic DNA is spliced and rejoined to juxtapose the VDJ elements to the C-region exons that encode the γ, ε and α chains of IgG, IgE and IgA isotypes, respectively; these C-region exons are located further downstream in the IgH region. This process results in an immunoglobulin gene that encodes an antibody of a different isotype.

### S Regions

The molecular basis of antibody class switching to the expression of C_{γ}, C_{ε}, and C_{α}, genes in activated B cells is a recombination which positions the new C_{H} gene 3′ next to the VDJ gene. The apparent sites of Ig class switch recombination are located within the S regions, highly repetitive DNA sequences which are present 5′ of each C_{H} gene, except C_{δ}.

All murine and most humans S regions are sequenced at least partially. They are 1-10 kb in length, highly repetitive and GC-rich. Murine and human S_{μ} are almost homogeneously composed of the two pentamer sequences GAGCT and GGGGT and the heptamer sequence (C/T)AGGTTG. All other S regions also contain multiple copies of the pentameric sequences. All murine S regions except S_{μ} are composed of tandem repeats that vary both in sequence and in length, with 49 bp repeats for S_{γ}1, S_{γ}3 and S_{γ}2b, 52 bp repeats for S_{γ}2a, 80 bp repeats for S_{α} and 40 bp repeats for S_{ε}. Both human and murine Sₒ are more homologous to S_{ε} and S_{α} than to the S_{γ} regions, which have considerable homology among each other. The S regions are sufficiently conserved between human and mouse to allow human S regions to be used as substrate for switch recombination in murine cells. The S_{μ}, S_{ε} and S_{α} regions are more homologous between the two species than the S_{γ} regions. Indeed, the Mouse Sm motif GGGCTGGGCTG (SEQ ID NO:1) is found in Se and a second motif GAGCTGACT is slightly modified in the Sε region as GAGCTGAGCT (has an added G relative to the Sm motif) (SEQ ID NO:2). The length of the S regions is subject to considerable allelic variation (length polymorphism) indicating that there is no functional requirement for a particular size of a given S region.

### IgE and Serum IgE Levels

Immunoglobulin E (IgE) is a class of antibody (or immunoglobulin “isotype”) that has only been found in mammals. It plays an important role in allergy, and is especially associated with type 1 hypersensitivity. IgE has also been implicated in immune system responses to most parasitic worms like Schistosoma mansoni, Trichinella spiralis, and Fasciola hepatica, and may be important during immune defense against certain protozoan parasites such as Plasmodium falciparum.

Although IgE is typically the least abundant isotype—blood serum IgE levels in a normal (“non-atopic”) individual are only 0.05% of the IgG concentration, compared to 10 mg/ml for the IgGs (the isotypes responsible for most of the classical adaptive immune response)—it is capable of triggering the most powerful immune reactions.

Atopic individuals can have up to 10 times the normal level of IgE in their blood (as do sufferers of hyper-IgE syndrome). IgE that can specifically recognise an “allergen” (typically this is a protein, such as dust mite DerP1, cat FelD1, grass or ragweed pollen, etc.) has a unique long-lived interaction with its high affinity receptor, FcER1, so that basophils and mast cells, capable of mediating inflammatory reactions, become “primed”, ready to release chemicals like histamine, leukotrienes and certain interleukins, which cause many of the symptoms we associate with allergy, such as airway constriction in asthma, local inflammation in eczema, increased mucus secretion in allergic rhinitis and increased vascular permeability, ostensibly to allow other immune cells to gain access to tissues, but which can lead to a potentially fatal drop in blood pressure as in anaphylaxis. Although the mechanisms of each response are fairly well understood, why some allergics develop such drastic sensitivities when others merely get a runny nose is still not well understood.

Total serum IgE concentration tests allows for measurement of the total IgE level in a serum sample. Elevated levels of IgE are associated with the presence of allergy. One method of testing for total serum IgE is the PRIST (paper radioimmunosorbent test). This test involves causing serum samples to react with IgE that has been tagged with radioactive iodine. Bound radioactive iodine, calculated upon completion of the test procedure, is proportional to the amount of total IgE in the serum sample. In clinical immunology, levels of individual classes of immunoglobulins are measured by nephelometry (or turbidimetry) to characterize the antibody profile of patient. Other methods of measuring IgE levels are ELISA, immunofluorescence, Western blot, immunodiffusion and immunoelectrophoresis.

Measurement of a total serum IgE concentration using a UniCAP 250® system (Pharmacia, Uppsala, Sweden) above 100 kU/L is considered elevated. In one study, using a sensitive double antibody radioimmunoassay to measure IgE, serum IgE from normal subjects free from evident allergic symptoms varied over a 130-fold range from 6 to 1000 ng/ml. In patients with allergic respiratory diseases the range of IgE concentrations overlapped that of normal subjects to a considerable extent, but approximately 35% of untreated allergic individuals had IgE concentrations above the 97^{th} percentile for normals and 51% are above the 95^{th} percentile. (See G. J. Gleich, A. K. Averbach and N. A. Swedlund, Measurement of IgE in normal and allergic serum by radioimmunoassay. J. Lab. Clin. Med. 77 (1971), p. 690.)

In another study, the geometric mean IgE level of normal adults was 105 ng/ml with a 95% interval of 5 to 2045. The normal level of IgE in adults has been reported to be approximately 100 to 400 μg/ml, 1/400,000 of that of IgG. (see Waldmann et al., The Journal of Immunology, 1972, 109: 304-310; see also Medical Immunology—10th Ed. (2001) TG Parslow, D P Stites, Al Terr and J B Imboden, eds., Table 7-2).

For a comparison of serum Ig levels in young (24-43 y.o.), old (66-96) and centenarians (99-108) see Listi et al., A Study of Serum Immunoglobulin Levels in Elderly Persons That Provides New Insights into B Cell Immunosenescence. Ann. N.Y. Acad. Sci. (2006) 1089:487-495. In particular see Table 2 of Listi et al. (supra) for the age- and gender-related serum concentration of immunoglobulins for normal individuals

Although it normally represents only a minute fraction (0.004%) of all serum antibodies, immunoglobulin E (IgE) is extremely important from the clinical standpoint because of its central involvement in allergic disorders. Two specialized types of inflammatory cells involved in allergic responses, the mast cell and the basophil, carry a unique, high-affinity Fc receptor that is specific for IgE antibodies. Thus, despite the very low concentration of IgE (roughly 10⁻⁷ M) in blood and tissue fluids, the surfaces of these cells are constantly decorated with IgE antibodies, adsorbed from the blood, that serve as antigen receptors. When its passively bound IgE molecules contact an antigen, the mast cell or basophil releases inflammatory mediator substances that produce many of the acute manifestations of allergic disease. Elevated levels of serum IgE may also signify infection by helminths or certain other types of multicellular parasites. Like IgG and IgD, IgE exists only in monomeric form. Fc receptors appear to recognize primarily the C_{H}3 domain of the E chain. See Medical Immunology—10th Ed. (2001; supra)

Despite the variability in the serum concentrations of immunoglobulin E in humans it is clear that serum IgE levels of greater than about 2500 μg/ml are associated with a variety of diseases. Similar low levels of IgE is reported in mice (see Pinaud et al., Localization of the 3′ IgH locus Elements that Effect Long-Distance Regulation of Class Switch Recombination, Immunity (2001) 15(2):187-199).

### Gene Targeting and Plasmid

Gene targeting is a technique utilizing homologous recombination between an engineered exogenous DNA fragment and the genome of the mouse embryonic stem (ES) cells. Recombination between identical regions contained within the introduced DNA fragment and the native chromosome will lead to the replacement of a portion of the chromosome with the engineered DNA. These modified ES cells can then be injected into blastocysts where they can incorporate and contribute to the fetal development along with the blastomeres from the ICM (inner cell mass).

In brief, gene targeting vectors are designed which, through homologous recombination, replace the wild-type allele of a given gene with a mutated form. The targeted ES cells are then implanted into 2-4 day blastocysts and transferred to pseudopregnant mothers (see below).

The targeting vectors used herein have four components:
a. a 5′ arm (also referred to as a 5′ flanking region);
b. a selection marker;
c. a DNA sequence encoding a donor switch region; and
d. a 3′ arm (also referred to as a 5′ flanking region).
   The 5′ arm is a fragment of DNA homologous to the 5′ end of the switch region to be replaced. The selection marker confers a selectable phenotype upon homologous recombination. The selection marker may be flanked by Ioxp sites. The donor switch region may be either before or after the selection marker. The 3′ arm is a fragment of DNA homologous to the 3′ end of the switch region to be replaced.

The 5′ and 3′ flanking regions may be any length but is dependent on the degree of the homology. As used herein “substantial homology” between two DNA sequence portions means that the sequence portions are sufficiently homologous to facilitate detectable recombination when DNA fragments are co-introduced into a recombination competent cell. Two sequence portions are substantially homologous if their nucleotide sequences are at least 40%, preferably at least 60%, more preferably at least 80% and most preferably, 100% identical with one another. This is because a decrease in the amount of homology results in a corresponding decrease in the frequency of successful homologous recombination. A practical lower limit to sequence homology can be defined functionally as that amount of homology which if further reduced does not mediate detectable homologous recombination of the DNA fragments in a recombination competent mammalian cell. The 5′ and 3′ flanking regions are preferably at least 500 bp, more preferably, 1000 bp, next most preferably about 1800 bp, and most preferably, greater than 1800 bp for each homologous sequence portion.

Desirably, a marker gene is used in the targeting construct to replace the deleted sequences. Various markers may be employed, particularly those which allow for positive selection. Of particular interest is the use of G418 resistance, resulting from expression of the gene for neomycin phosphotransferase (“neo”). The presence of the marker gene in the genome will indicate that integration has occurred.

The donor switch region may be the Sμ, Sγ1, Sγ2a, Sγ2b or Sγ3 region when the Sε region is the region to be replaced. The donor region should be one that under stimulated, non-recombinant conditions (i.e., the switch regions have not been altered) results in its associated heavy chain is expressed at a higher level than Cc.

For the most part, DNA analysis by Southern blot hybridization will be employed to establish the location of the integration. By employing probes for the insert and the sequences at the 5′ and 3′ regions flanking the region where homologous integration would occur, one can demonstrate that homologous targeting has occurred.

PCR may also be used with advantage in detecting the presence of homologous recombination. PCR primers may be used which are complementary to a sequence within the targeting construct and complementary to a sequence outside the construct and at the target locus. In this way, one can only obtain DNA molecules having both the primers present in the complementary strands if homologous recombination has occurred. By demonstrating the expected size fragments, e.g. using Southern blot analysis, the occurrence of homologous recombination is supported.

Once a targeting construct has been prepared and any undesirable sequences removed, e.g., procaryotic sequences, the construct may now be introduced into the target cell, for example an ES cell (see below). Any convenient technique for introducing the DNA into the target cells may be employed. Techniques include protoplast fusion, e.g. yeast spheroplast:cell fusion, lipofection, electro-poration, calcium phosphate-mediated DNA transfer or direct microinjection.

After transformation or transfection of the target cells, target cells may be selected by means of positive and/or negative markers, as previously indicated, neomycin resistance and acyclovir or gancyclovir resistance. Those cells which show the desired phenotype may then be further analyzed by restriction analysis, electrophoresis, Southern analysis, PCR, or the like. By identifying fragments which show the presence of the desired alteration at the target locus, one can identify cells in which homologous recombination has occurred to alter the IgH in a manner that enhances switching to Cε.

### Embryonic Stem (ES) Cell Methods

A. Introduction of cDNA into ES cells

Methods for the culturing of ES cells and the subsequent production of recombinant animals, the introduction of DNA into ES cells by a variety of methods such as electroporation, calcium phosphate/DNA precipitation, and direct injection are described in detail in Teratocarcinomas and embryonic stem cells, a practical approach, ed. E. J. Robertson, (IRL Press 1987), the teachings of which are incorporated herein. Selection of the desired clone of recombinant ES cells is accomplished through one of several means. In cases involving sequence specific gene integration, a nucleic acid sequence for recombination with the gene of interest or sequences for controlling expression thereof is co-precipitated with a gene encoding a marker such as neomycin resistance. Transfection is carried out by one of several methods described in detail in Lovell-Badge, in Teratocarcinomas and embryonic stem cells, a practical approach, ed. E. J. Robertson, (IRL Press 1987) or in Potter et al., Proc. Natl. Acad. Sci. USA 81, 7161 (1984). Calcium phosphate/DNA precipitation, direct injection, and electroporation are the preferred methods. In these procedures, a number of ES cells, for example, 0.5×10⁶, are plated into tissue culture dishes and transfected with a mixture of the linearized nucleic acid sequence and 1 mg of pSV2neo DNA (Southern and Berg, J. Mol. Appl. Gen. 1:327-341 (1982)) precipitated in the presence of 50 mg lipofectin in a final volume of 100 μl. The cells are fed with selection medium containing 10% fetal bovine serum in DMEM supplemented with an antibiotic such as G418 (between 200 and 500 μg/ml). Colonies of cells resistant to G418 are isolated using cloning rings and expanded. DNA is extracted from drug resistant clones and Southern blotting experiments using the nucleic acid sequence as a probe are used to identify those clones carrying the desired nucleic acid sequences. In some experiments, PCR methods are used to identify the clones of interest.

DNA molecules introduced into ES cells can also be integrated into the chromosome through the process of homologous recombination, described by Capecchi, (1989) Science 244:1288-1292. Direct injection results in a high efficiency of integration. Desired clones are identified through PCR of DNA prepared from pools of injected ES cells. Positive cells within the pools are identified by PCR subsequent to cell cloning (Zimmer and Bruss, Nature 338, 150-153 (1989)). DNA introduction by electroporation is less efficient and requires a selection step. Methods for positive selection of the recombination event (i.e., neo resistance) and dual positive-negative selection (i.e., neo resistance and ganciclovir resistance) and the subsequent identification of the desired clones by PCR have been described by Joyner et al., Nature 338, 153-156 (1989) and Capecchi, (1989), the teachings of which are incorporated herein.

### B. Embryo Recovery and ES Cell Injection

Female animals are induced to superovulate using methodology adapted from the standard techniques used with mice, that is, with an injection of pregnant mare serum gonadotrophin (PMSG; Sigma) followed 48 hours later by an injection of human chorionic gonadotrophin (hCG; Sigma). Females are placed with males immediately after hCG injection. Approximately one day after hCG, the mated females are sacrificed and embryos are recovered from excised oviducts and placed in Dulbecco's phosphate buffered saline with 0.5% bovine serum albumin (BSA; Sigma). Surrounding cumulus cells are removed with hyaluronidase (1 mg/ml). Pronuclear embryos are then washed and placed in Earle's balanced salt solution containing 0.5% BSA (EBSS) in a 37.5° C. incubator with a humidified atmosphere at 5% CO₂, 95% air until the time of injection.

Naturally cycling or superovulated females mated with males are used to harvest embryos for the injection of ES cells. Embryos of the appropriate age are recovered after successful mating. Embryos are flushed from the uterine horns of mated females and placed in Dulbecco's modified essential medium plus 10% calf serum for injection with ES cells. Approximately 10-20 ES cells are injected into blastocysts using a glass microneedle with an internal diameter of approximately 20 μm.

### C. Transfer of Embryos to Pseudopregnant Females

Randomly cycling adult females are paired with vasectomized males. Recipient females are mated such that they will be at 2.5 to 3.5 days post-mating (for mice, or later for larger animals) when required for implantation with blastocysts containing ES cells. At the time of embryo transfer, the recipient females are anesthetized. The ovaries are exposed by making an incision in the body wall directly over the oviduct and the ovary and uterus are externalized. A hole is made in the uterine horn with a needle through which the blastocysts are transferred. After the transfer, the ovary and uterus are pushed back into the body and the incision is closed by suturing. This procedure is repeated on the opposite side if additional transfers are to be made.

The procedures for manipulation of the embryo and for microinjection of DNA are described in detail in Hogan et al., Manipulating the mouse embryo, Cold Spring Harbor laboratory, Cold Spring Harbor, N.Y. (1986), the teachings of which are incorporated herein. These techniques are readily applicable to embryos of other animal species, and, although the success rate is lower, it is considered to be a routing practice to those skilled in this art.

### D. Identification of Recombinant Animals

Samples (1-2 cm of mouse tails) are removed from young animals. For larger animals, blood or other tissue can be used. To test for chimeras in the homologous recombination experiments, i.e., to look for contribution of the targeted ES cells to the animals, coat color has been used in mice, although blood could be examined in larger animals. DNA is prepared and analyzed by both Southern blot and PCR to detect recombinant founder (F₀) animals and their progeny (F₁ and F₂).

Once the recombinant animals are identified, lines are established by conventional breeding.

### Southern Analysis

DNA was obtained from cell lines by standard phenol extraction procedure or by cesium gradient centrifugation.

### A. Phenol Extraction

Flasks of cells are washed with HBSS buffer, then 2.5 ml/100 cm² of lysing solution (1% sodium dodecyl sulfate/150 mM NaCl/10 mM EDTA/10 mM Tris, pH 7.4) is added. After all cells are solubilized, they are transferred to a 50 ml conical tube and proteinase K to a final concentration of 0.4 mg/ml is added. The lysate is incubated at 65° C. for 10 minutes to inactivate DNAse enzymes, then incubated overnight at 37° C. To this lysate an equal volume of fresh phenol that has been equilibrated in 50 mM Tris, pH 8.0, is added, and the tube is gently inverted for 5 minutes at room temperature (about 22°-24° C.) then centrifuged at 2000 g for 5 minutes, and the top (aqueous) layer is transferred to a second tube. An equal volume of 50% phenol/50% chloroform (v/v) is then added, and the inversion centrifugation process repeated. The supernatant is then transferred to a third tube, and an equal volume of chloroform is added. After a third inversion, centrifugation cycle, the supernatant is transferred to a fourth tube and the DNA precipitated by the addition of 1/10 volume of 3M NaAcetate, 2.5 vol of cold ethanol. After washing the resulting precipitate with 70% ethanol and air-drying the pellet, it is resuspended in TE buffer (10 mM Tris pH 7.4/1 mM EDTA) and RNase to a final concentration of 50% μg/ml is added (The RNase is prepared to be DNase-free by heating the freshly suspended enzyme at 70° C. for 30 minutes. The solution is then extracted with an equal volume of 1:1 SS-phenol:chloroform. The phases are separated by centrifugation, as above, and the supernatant extracted with an equal volume of chloroform. Following centrifugation, as above, and the supernatant extracted with an equal volume of chloroform. Following centrifugation, the DNA in the supernatant is precipitated with 12.5 ml of ethanol, then washed with 70% ethanol and air-dried. The pellet is then suspended in TE buffer, and the DNA yield determined by O.D. reading at 260 nM and the purity determined by 260/280 ratio. The DNA preparation is stored in TE at 4° C.

B. Cesium Chloride Preparation of RNA and DNA from Cultured Cells

Flasks of cells were washed with HBS then 2.5 ml/100 cm² of guanidine isothyocyanate (GIT) buffer was added. The guanidine isothyocyanate buffer was 4M GIT/25 mM sodium acetate, pH6/0.8% beta-mercaptoethanol (v/v). After 3-5 minutes, with gentle rocking, the cell lysates were layered on top of 4 ml of cesium chloride buffer in Beckman SW41 10 ml ultracentrifuge tubes. The tubes were filled to nearly the top with GIT buffer, then they were spun overnight at 32,000 rpm (174,000×g) at 200 C. The GIT solution in the upper two-thirds of the tube was then removed and discarded, the CsCl solution in the lower one third of the tube that contains the DNA was transferred to a second tube. The RNA pellet in the bottom of the rube was resuspended in 200 μl of 0.3 M sodium acetate, pH 6 and transferred to a 1.5 ml microfuge tube. To this tube was added 750 μl of ethanol, and the tube was placed on dry ice for 10 minutes. After microcentrifugation for 10 min., the supernatant was discarded, 300 μl of 70% ethanol was added, and the tube was microfuged again. The supernatant was discarded, and the pellet was dried in a vacuum centrifuge. The pellet was resuspended in 200 μl of dH₂O. The RNA preparation was stored as an ethanol precipitate of −70° C. The 4 ml of CsCl containing the DNA was diluted with dH₂O. To this was added 30 ml of cold ethanol. The DNA precipitate was recovered, transferred to a new 50 ml tube, and rinsed with 70% ethanol, then air-dried. The pellet was then resuspended in PK buffer, and 10 mg of proteinase K was added. After incubation at 65° C. for 15 minutes, the solution was incubated overnight at 37° C. The hydrolysate was then extracted with 1:1 SS-phenol:chloroform, followed by chloroform, ethanol precipitation, and quantitated as described above.

### C. Restriction Digestion, Electrophoresis, and Southern Transfer

Restriction endonuclease digest conditions were according to the recommendations of the suppliers. For genomic DNA, the restriction digestion was for 4-6 hrs. at 37° C. For simple DNA preparations (cloned or PCR amplified) the incubation was for 1-2 hours at 37° C. Generally, 10 μg of DNA was digested in a volume of 150 μl. The digest was precipitated by addition of 3 μl 5M NaCl and 375 μl (2.5 vol) of cold ethanol, microfuged for 10 minutes at 4° C., washed with 500 μl cold 70% ethanol and microfuged.

The pellet was air-dried in a vacuum microfuge and resuspended in 17 μl of electrophoresis running buffer (routinely TAE buffer) and 3 μl of gel loading buffer (TAE buffer containing 50% glycerol/1% saturated bromphenol blue), heated to 68° C. for 10 minutes, and loaded into wells of an agarose gel, along with a lambda-HindIII digest in a separate well to serve as a size marker. The concentration of agarose in the gel was 1.0%. Following electrophoresis for 8-16 hours, the gel was stained with ethidium bromide, the migration distance of the marker bands measured and recorded, and the gel photographed.

The digested DNA was vacuum-transferred to a Nytran membrane. The gel was laid on top of the Nytran membrane on the vacuum apparatus, covered with 500 ml of 0.4 M NaOH/0.8 M NaCl and a vacuum pressure of 50 cm of water applied for four minutes. The NaCl—NaOH solution was removed, 500 ml of 10×SSC added, and a pressure of 50 cm water applied for 30-60 minutes. The Southern blot was then baked at 80° C. for 2 hours and stored in a vegetable freezing bag.

### D. Southern Hybridization

The Southern blot was placed in a heat sealable plastic bag and incubated with 10 ml of pre-hybridization buffer containing 1 M NaCl, 1% SDS, 10% dextran sulphate, and 200 μg/ml herring sperm DNA, and incubated for 15 minutes at 65° C. A corner of the bag was then cut off, and the radiolabelled oligonucleotide probe was added (approximately 10⁷ dpm). The bag was resealed and placed at 65° C. in an oven or water bath and gently rocked or shaken for 12-16 hours. The membrane was then removed from the bag and washed in a series of increasingly dilute and higher temperature (increasing stringency) SSC buffers until the background radioactivity was low relative to the specifically bound probe. In a darkroom, the membrane was then placed in a plastic bag which was positioned in an X-ray film cassette equipped with intensifier screens, a sheet of Kodak XAR-5 film was added, and the sealed cassette was placed at −70° C. for variable time depending on the intensity of signal. Usually, exposures after varying time periods are useful. The film was developed in a Kodak X-OMAT automatic developer. Membranes may be re-hybridized several times. Nytran membranes may be stripped of labeled probe by heating in boiling 0.1×SSC for 2 minutes.

### B-Cell Culture

B cells may be purified from spleens by negative selection. Briefly, T cells in single cell suspensions are coated with antibodies and depleted by complement lysis. The remaining spleen cells were layered over a discontinuous Percoll (GE Healthcare) gradient. Resting B cells may be selected from the 66% to 70% interface and total B cells (50-70% Percoll interface) were used. B cells may be cultured in B cell media consisting of RPMI 1640 media (Sigma Aldrich), supplemented with 10% heat inactivated FBS, 100 U/ml penicillin and streptomycin, 2 mM L-glutamine, 1 mM sodium pyruvate, 10 mM Hepes, 100 mM non-essential amino acids and 5×10⁻⁵M 2-mercaptoethanol.

### Confirming In Vivo Hyper-IgE Production

Recombinant animals will be tested for elevated IgE serum levels using techniques known in the art. For example, the ImmunoCAP Specific IgE blood test (which the literature may also describe as: CAP RAST, CAP FEIA (fluorenzymeimmunoassay), and Pharmacia CAP) may be used.

Other methods are known in the art. Such methods include for example Enyzme-linked immunosorbent assay and potentially FACS using an anti-IgE antibody. Enzyme-Linked ImmunoSorbent Assay, also called ELISA, Enzyme ImmunoAssay or EIA, is a biochemical technique used mainly in immunology to detect the presence of an antibody or an antigen in a sample. Fluorescence-activated cell sorting (FACS) is a powerful technique for analyzing large mixed populations of single cells. A higher proportion of IgE positive cells would indicate an elevated serum IgE level.

### Hybridoma Production

Standard techniques known in the art are used to prepare hybridomas that produce IgE. See, for example, Köhler & Milstein (1975) Continuous cultures of fused cells secreting antibody of predefined specificity, Nature 256:495, and Köhler & Milstein (1976) Derivation of specific antibody-producing tissue culture and tumor lines by cell fusion. Eur. J. Immunol. 6:511.

Briefly, immunized splenocytes are washed and fused to myeloma cells under appropriate conditions. The hybridomas are exposed to HAT or other selection agent 24 hours later, and the non-fused myeloma cells will die. The non-fused splenocytes also have a finite lifetime, and the hybridomas are then the only proliferating cells left in the culture.

### Assay for Class Switching

Assays are known in the art and are described in, for example, Shinkura, R. et al. Nat. Immunol. (2003) 4, 435-441 and Zarrin, et al., Nat. Immunol. (2004) 5, 1275-1281. Briefly, splenocytes were stimulated with anti-CD40 and IL-4 for 4 days to generate hybridomas or for 6 days to perform ELISA. A monoclonal anti-IgE antibody may be used to detect IgE (mutated alleles). Total IgE may be measured by polyclonal anti-IgE antibodies (Southern Biotechnology Associates).

See also, for example, Southern and Berg (Detection of specific sequences among DNA fragments separated by gel electrophoresis. J Mol Appl Genet (1982) 1: 327-341) describes Southern blot analysis to assess DNA rearrangement and CSR in IgH locus.

ε Germline transcription marks the first step in the commitment of B cells to the synthesis of IgE. Therefore, RT-PCR may be used to examine E immunoglobulin heavy-chain germline gene transcripts (GLTs; ε GLTs), εcircle transcripts (CTs; I ε −Cμ CT or IεCγ CT), and mRNA encoding the heavy chain of IgE (c mRNA) and activation-induced cytidine deaminase (AID) (see Takhar et al., J Allergy Clin Immunol (2007) 119(1):213-218).

In the experimental disclosure which follows, the following abbreviations apply: eq (equivalents); M (Molar); μM (micromolar); N (Normal); mol (moles); mmol (millimoles); μmol (micromoles); nmol (nanomoles); g (grams); mg (milligrams); kg (kilograms); μg (micrograms); L (liters); ml (milliliters); μl (microliters); cm (centimeters); mm (millimeters); μm (micrometers); nm (nanometers); ° C. (degrees Centigrade); h (hours); min (minutes); sec (seconds); msec (milliseconds); Ci (Curies) mCi (milliCuries); μCi (microcuries); TLC (thin layer chromatography).

### EXAMPLES

The present invention is described in further detain in the following examples which are not in any way intended to limit the scope of the invention as claimed. The attached Figures are meant to be considered as integral parts of the specification and description of the invention. All references cited are herein specifically incorporated by reference for all that is described therein. The following examples are offered to illustrate, but not to limit the claimed invention.

### Example 1

### Gene Targeting/Generation of Mutant Mice

This example illustrates the construction of the targeting vector, the transformation of embryonic stem cells (ES) and generation of mutant mice.

### I. Generalized Procedure

A. Transformation of Embryonic Stem Cells (ES)

Targeting constructs were designed based on sequence information available in the NCBI for NT_{—}166318. (See also, Waterston et al., Initial sequencing and comparative analysis of the mouse genome, Nature. (2002) 420(6915):520-62.) A BamHI/PVuI fragment (7022 bp with Sε region (129 mice); 7355 bp with Sε region (B6 mice)) was isolated from 129 or C57B6 BAC clones and amplified.

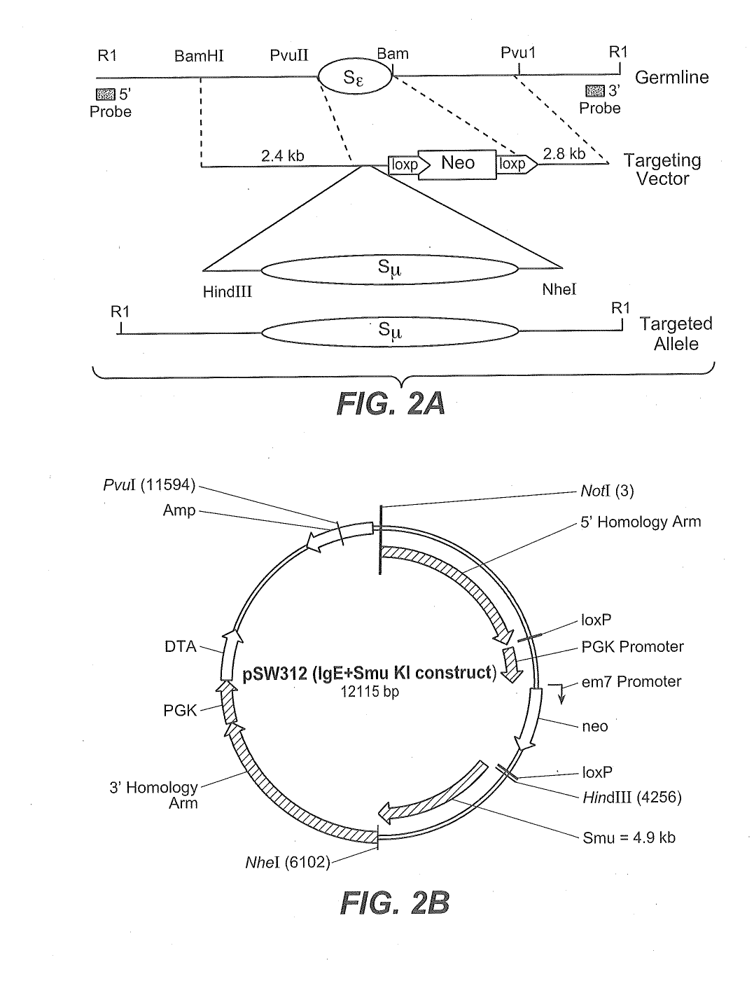

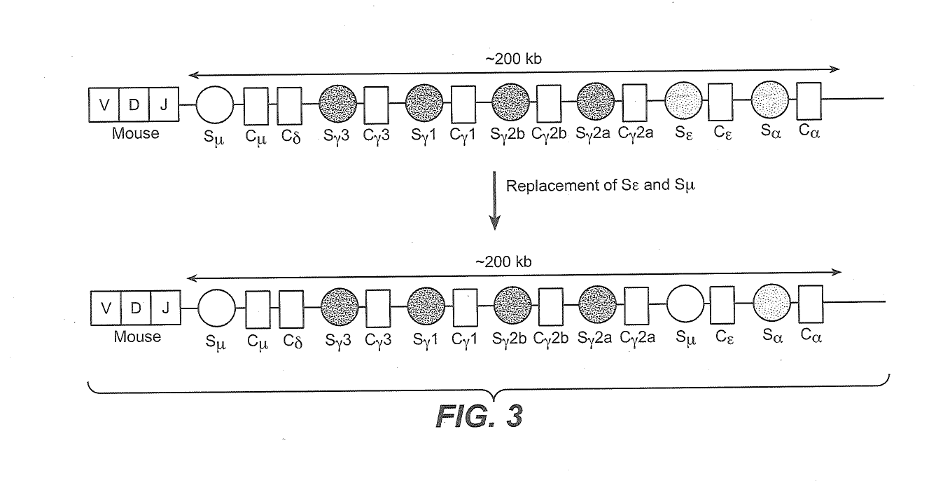

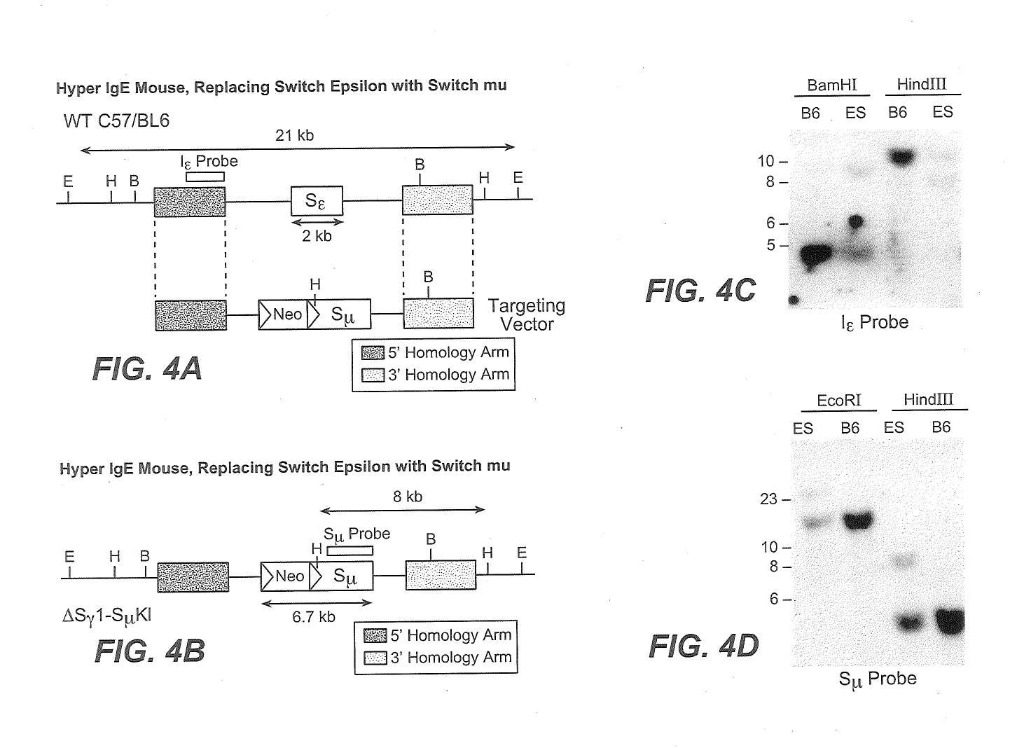

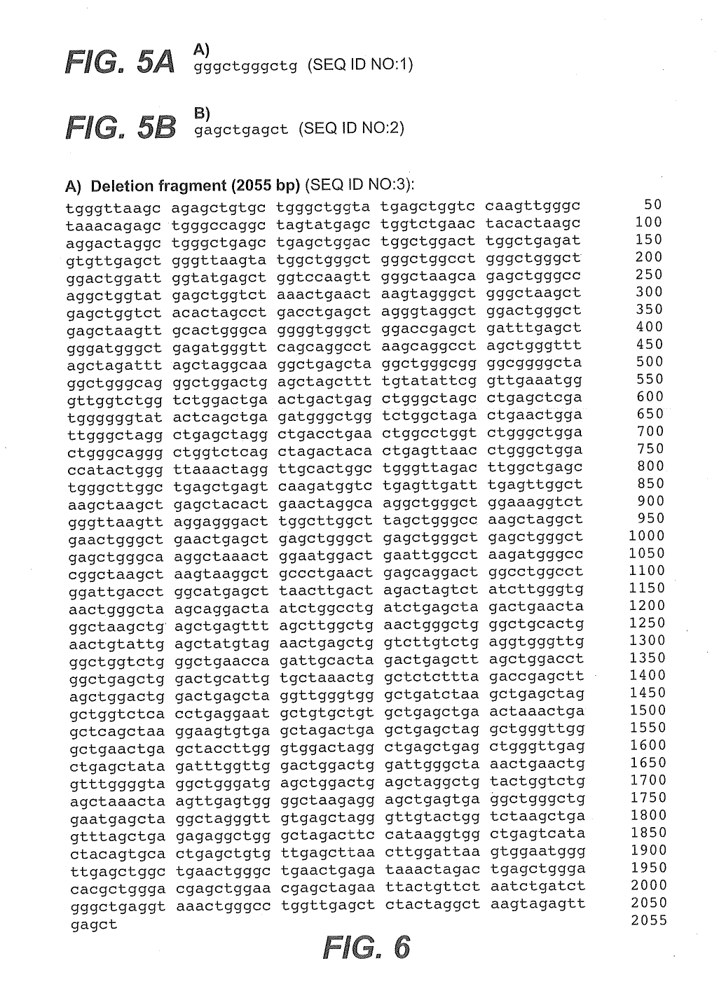

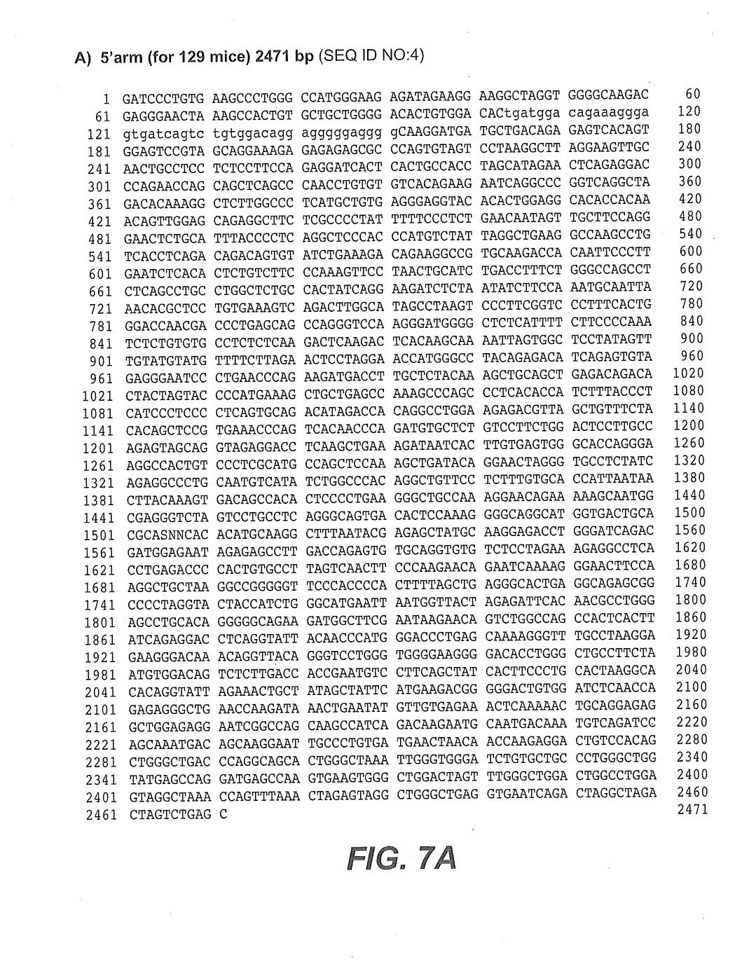

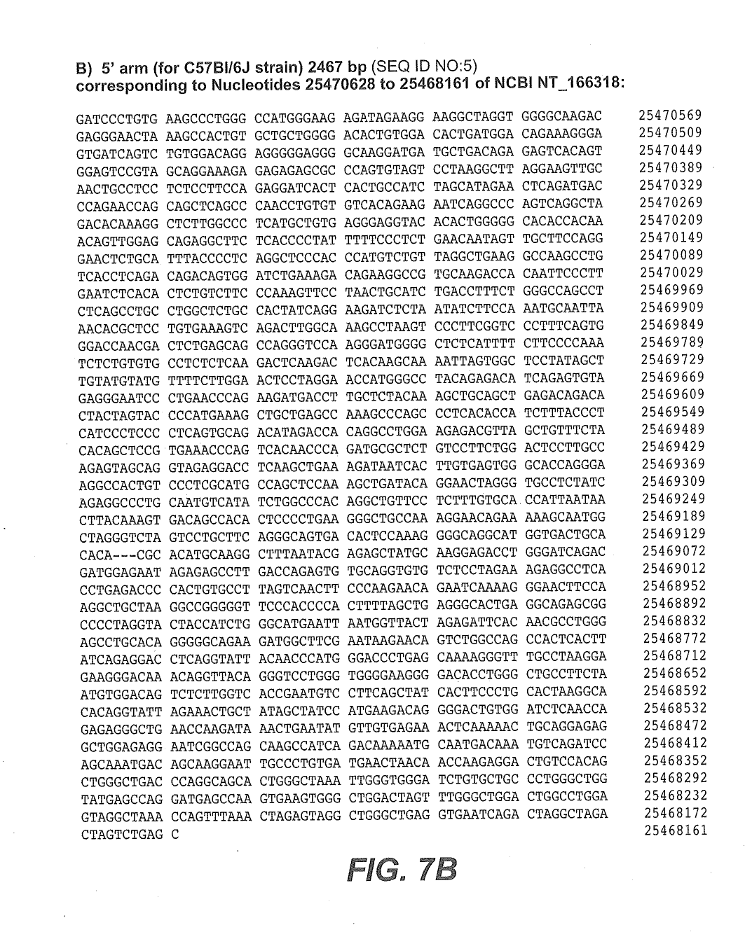

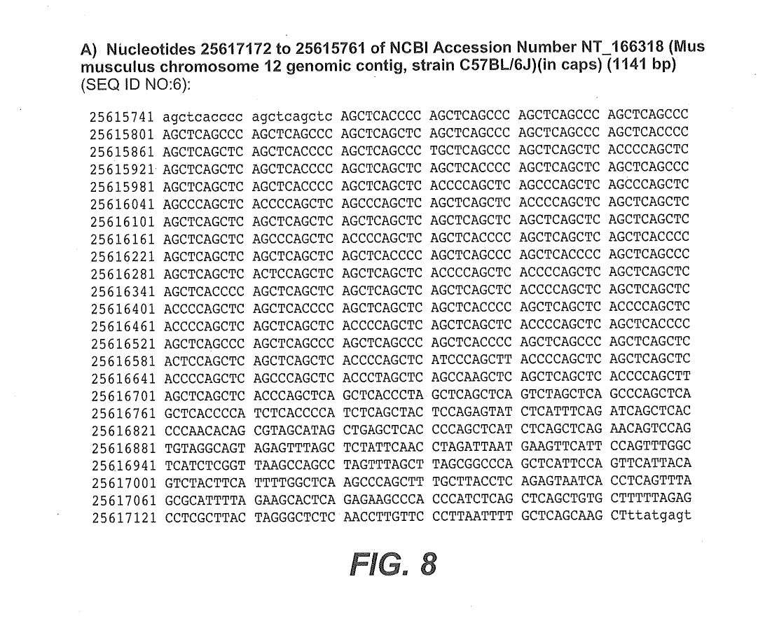

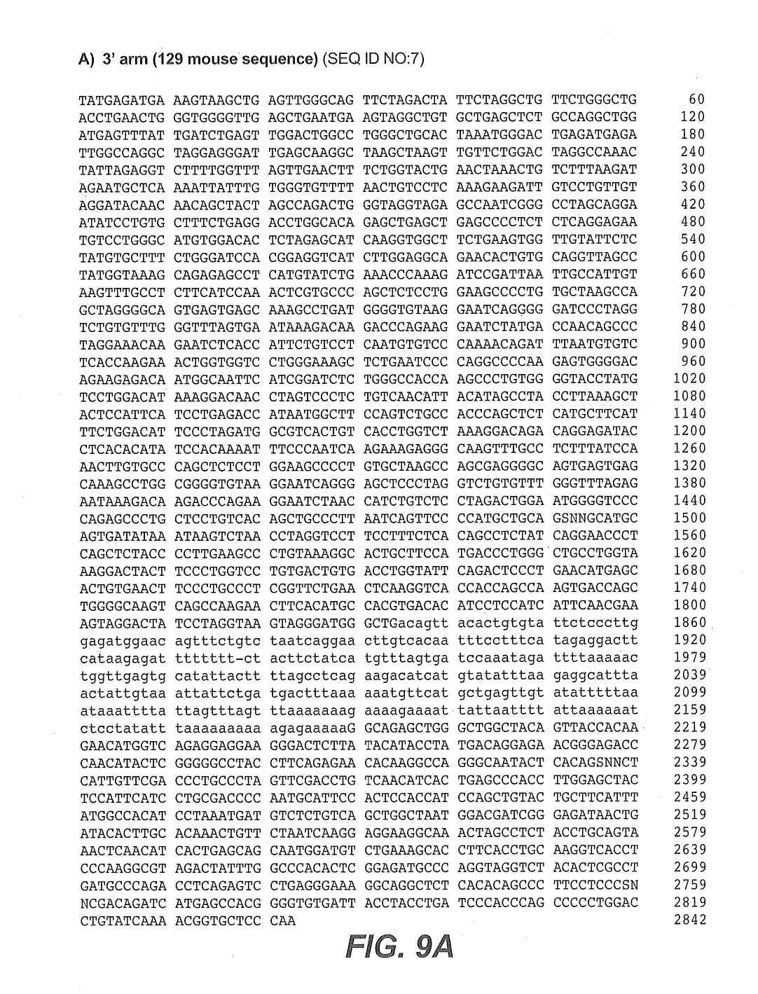

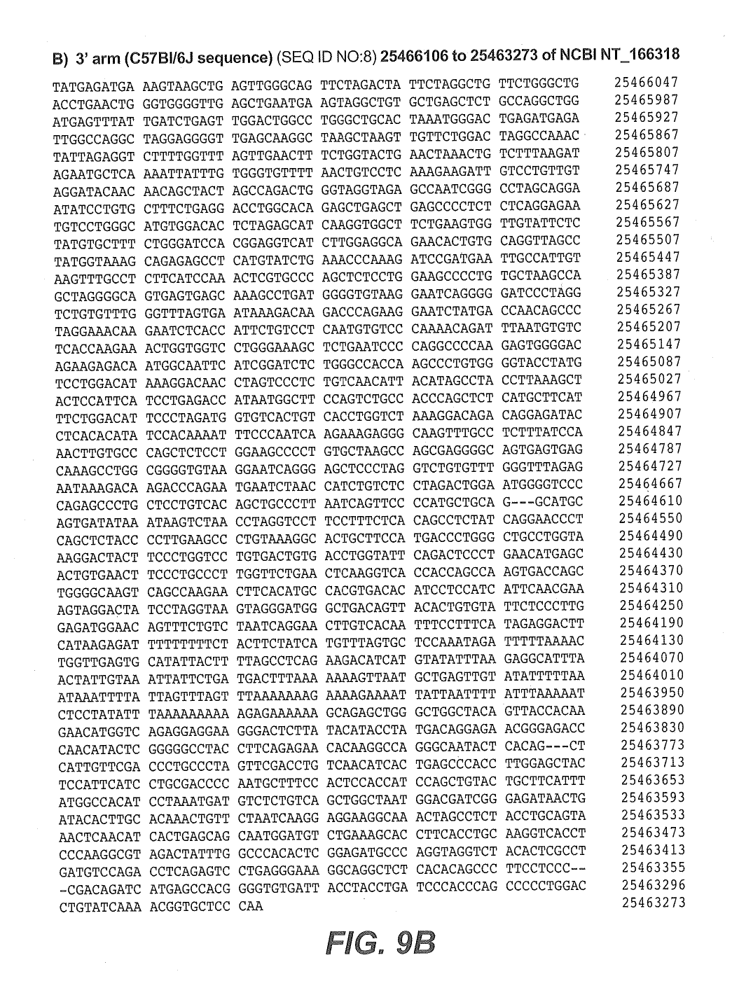

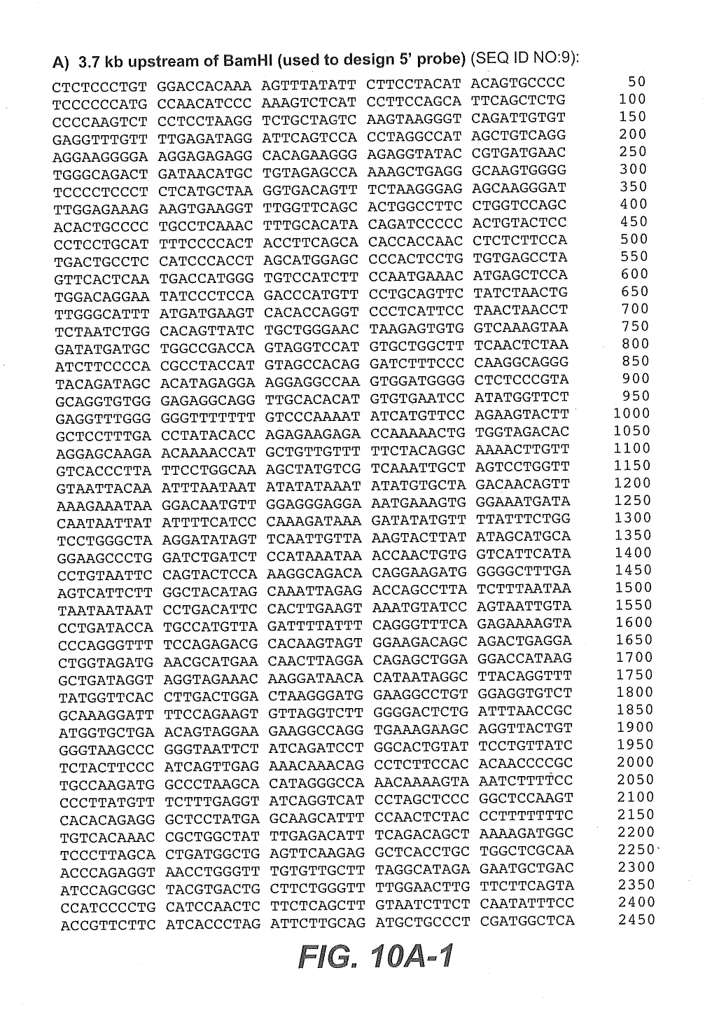

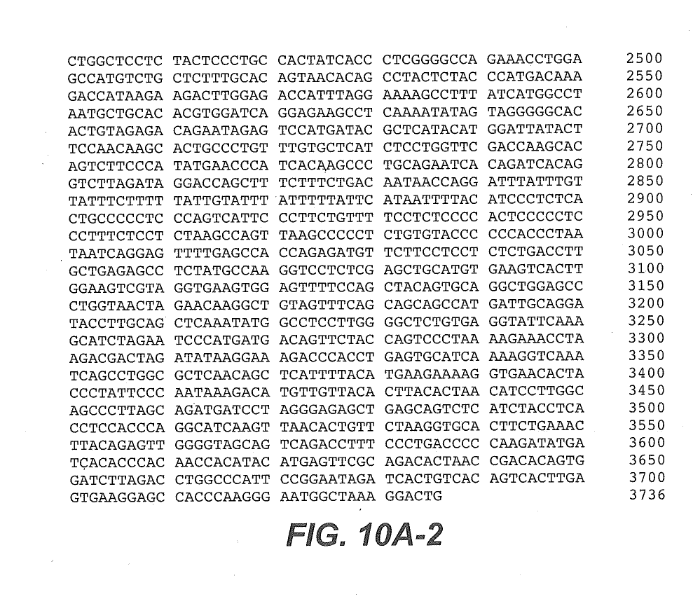

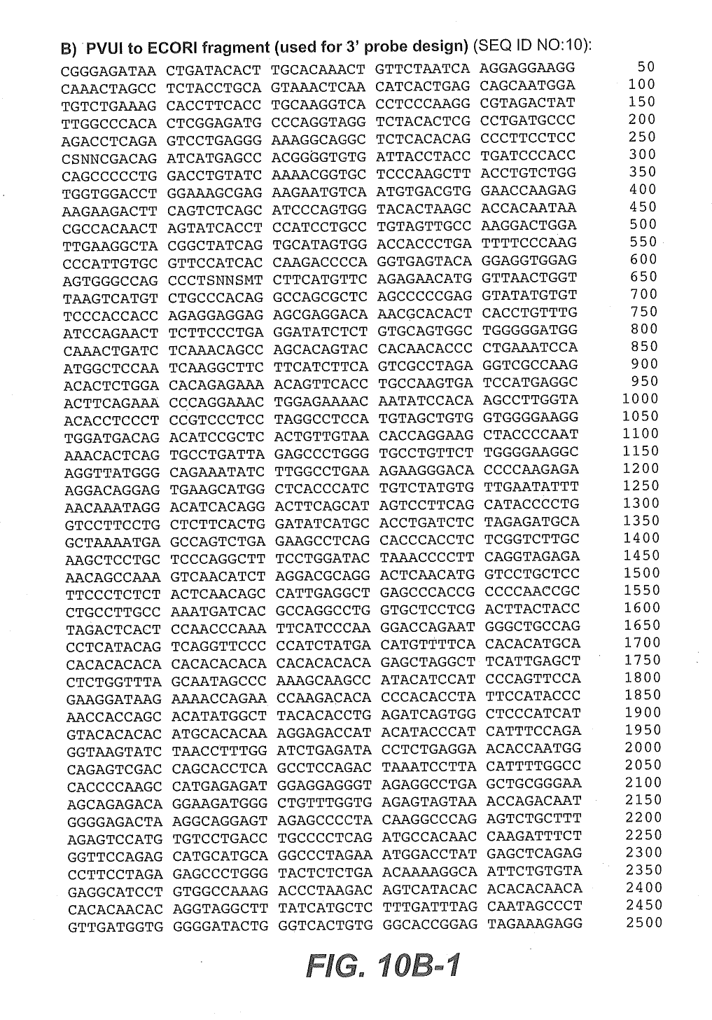

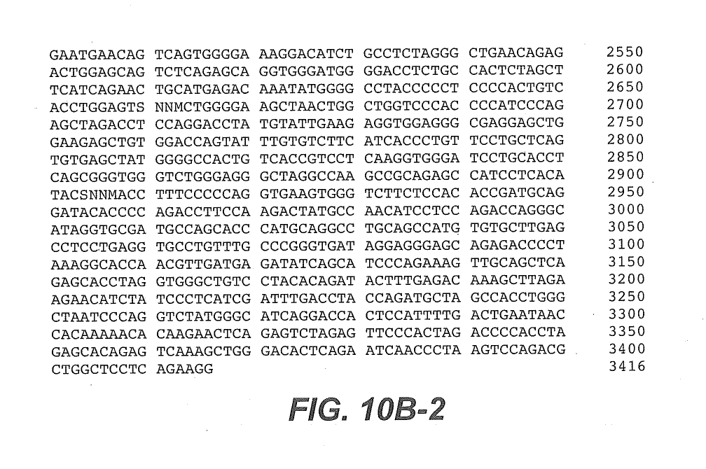

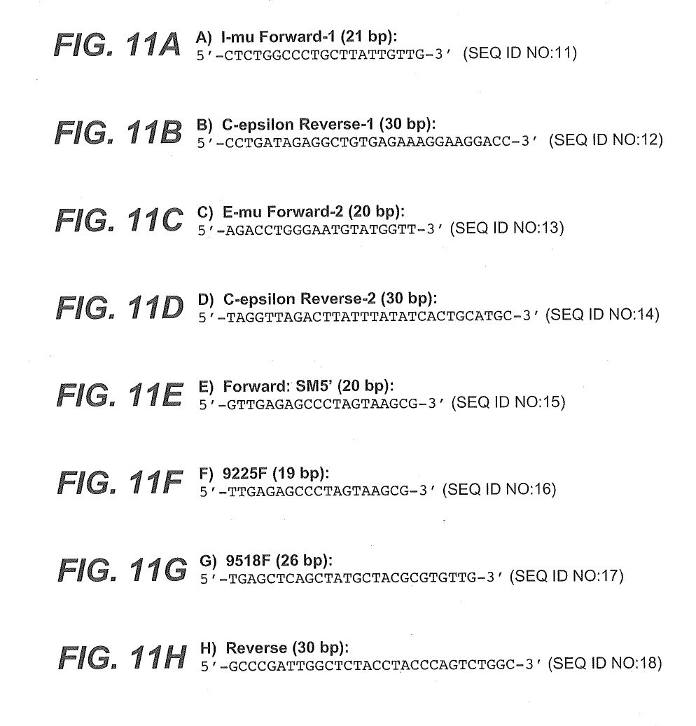

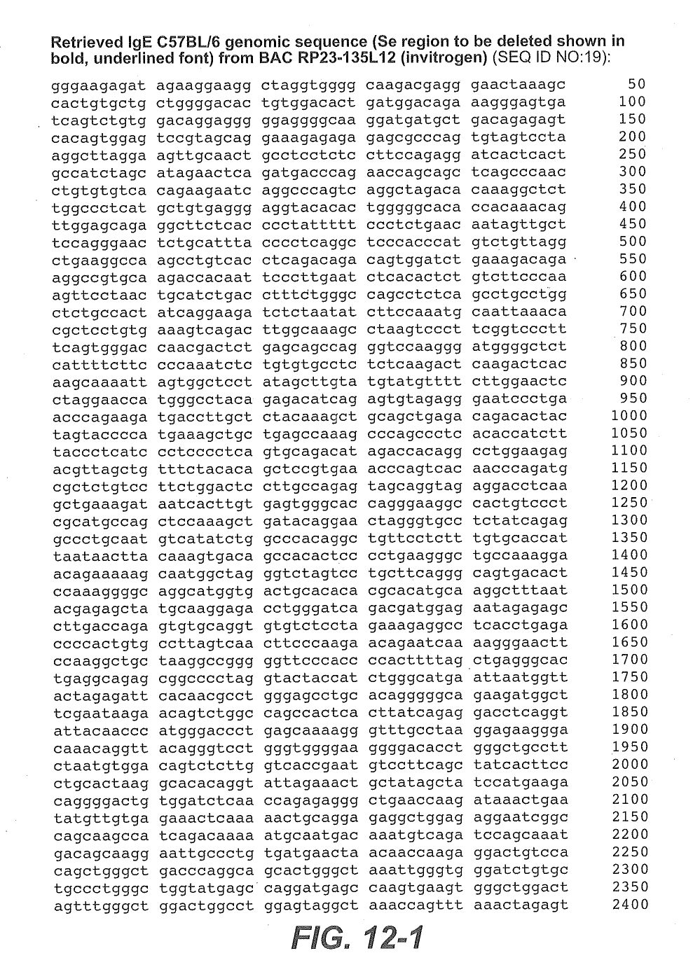

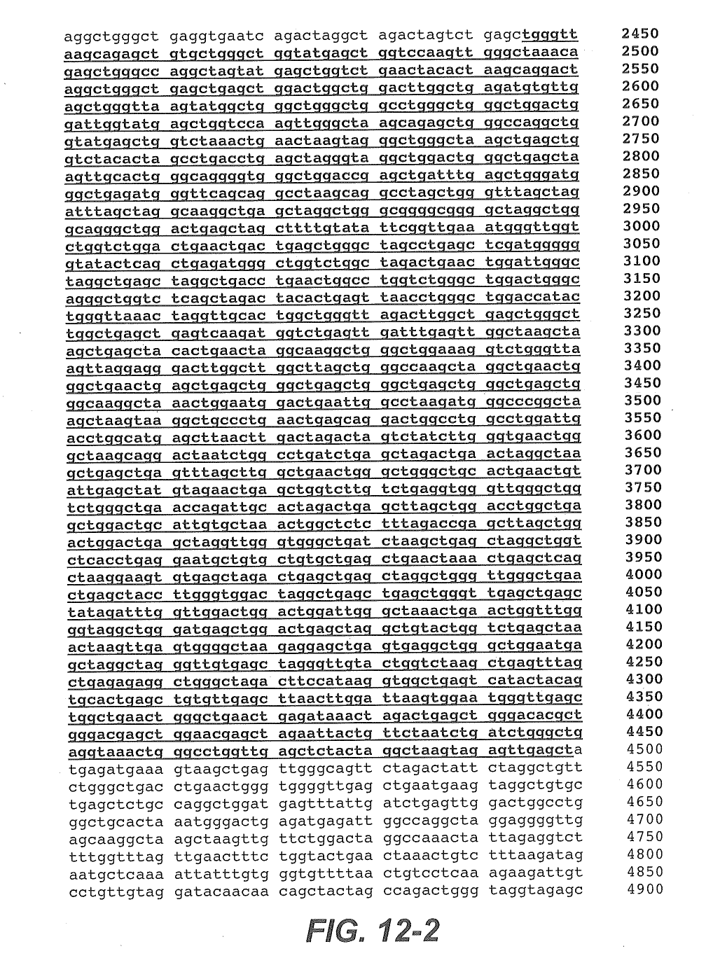

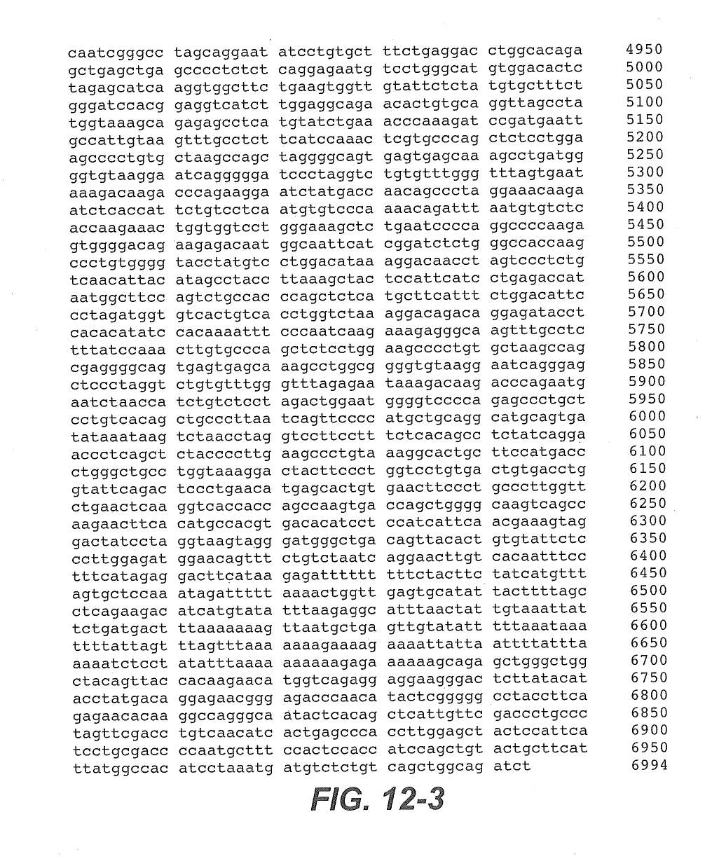

The following sequence (5′→3′; SEQ ID NO:3) was deleted from the BamHI/PVuI fragment and was replaced with a Neo cassette plus the HindIII/NheI containing most of mouse Sm region (see FIG. 2 and SEQ ID NO:6).

| | |
|---|---|
| tgggttaagc agagctgtgc tgggctggta tgagctggtc caagttgggc | 50 |
| taaacagagc tgggccaggc tagtatgagc tggtctgaac tacactaagc | 100 |
| aggactaggc tgggctgagc tgagctggac tggctggact tggctgagat | 150 |
| gtgttgagct gggttaagta tggctgggct gggctggcct gggctgggct | 200 |
| ggactggatt ggtatgagct ggtccaagtt gggctaagca gagctgggcc | 250 |
| aggctggtat gagctggtct aaactgaact aagtagggct gggctaagct | 300 |
| gagctggtct acactagcct gacctgagct agggtaggct ggactgggct | 350 |
| gagctaagtt gcactgggca ggggtgggct ggaccgagct gatttgagct | 400 |
| gggatgggct gagatgggtt cagcaggcct aagcaggcct agctgggttt | 450 |
| agctagattt agctaggcaa ggctgagcta ggctgggcgg ggcggggcta | 500 |
| ggctgggcag ggctggactg agctagcttt tgtatattcg gttgaaatgg | 550 |
| gttggtctgg tctggactga actgactgag ctgggctagc ctgagctcga | 600 |
| tggggggtat actcagctga gatgggctgg tctggctaga ctgaactgga | 650 |
| ttgggctagg ctgagctagg ctgacctgaa ctggcctggt ctgggctgga | 700 |
| ctgggcaggg ctggtctcag ctagactaca ctgagttaac ctgggctgga | 750 |
| ccatactggg ttaaactagg ttgcactggc tgggttagac ttggctgagc | 800 |
| tgggcttggc tgagctgagt caagatggtc tgagttgatt tgagttggct | 850 |
| aagctaagct gagctacact gaactaggca aggctgggct ggaaaggtct | 900 |
| gggttaagtt aggagggact tggcttggct tagctgggcc aagctaggct | 950 |
| gaactgggct gaactgagct gagctgggct gagctgggct gagctgggct | 1000 |
| gagctgggca aggctaaact ggaatggact gaattggcct aagatgggcc | 1050 |
| cggctaagct aagtaaggct gccctgaact gagcaggact ggcctggcct | 1100 |
| ggattgacct ggcatgagct taacttgact agactagtct atcttgggtg | 1150 |
| aactgggcta agcaggacta atctggcctg atctgagcta gactgaacta | 1200 |
| ggctaagctg agctgagttt agcttggctg aactgggctg ggctgcactg | 1250 |
| aactgtattg agctatgtag aactgagctg gtcttgtctg aggtgggttg | 1300 |
| ggctggtctg ggctgaacca gattgcacta gactgagctt agctggacct | 1350 |
| ggctgagctg gactgcattg tgctaaactg gctctcttta gaccgagctt | 1400 |
| agctggactg gactgagcta ggttgggtgg gctgatctaa gctgagctag | 1450 |
| gctggtctca cctgaggaat gctgtgctgt gctgagctga actaaactga | 1500 |
| gctcagctaa ggaagtgtga gctagactga gctgagctag gctgggttgg | 1550 |
| gctgaactga gctaccttgg gtggactagg ctgagctgag ctgggttgag | 1600 |
| ctgagctata gatttggttg gactggactg gattgggcta aactgaactg | 1650 |
| gtttggggta ggctgggatg agctggactg agctaggctg tactggtctg | 1700 |
| agctaaacta agttgagtgg ggctaagagg agctgagtga ggctgggctg | 1750 |
| gaatgagcta ggctagggtt gtgagctagg gttgtactgg tctaagctga | 1800 |
| gtttagctga gagaggctgg gctagacttc cataaggtgg ctgagtcata | 1850 |
| ctacagtgca ctgagctgtg ttgagcttaa cttggattaa gtggaatggg | 1900 |
| ttgagctggc tgaactgggc tgaactgaga taaactagac tgagctggga | 1950 |
| cacgctggga cgagctggaa cgagctagaa ttactgttct aatctgatct | 2000 |
| gggctgaggt aaactgggcc tggttgagct ctactaggct aagtagagtt | 2050 |
| gagct | 2055 |

A similar construct may be made to target 129SvEv ES cells to account for strain allelic differences that exist in the IgH locus.

B. Transformation of Embryonic Stem Cells (ES)

The plasmid from above was linearized using PvuI restriction enzyme. DNA was washed in 70% Ethanol and subsequently pelleted and resuspened in 50 μl TE. Using techniques known in the art (see, for example, Templeton et al., Efficient gene targeting in mouse embryonic stem cells, Gene Therapy (1997) 4:700-709), 10⁶ ES cells were transfected with linearized vector by electroporation and selected using G418 (400 μg/ml). Subsequently the Neo gene was deleted using cre/loxP recombination system. Correctly targeted clones were identified by Southern analyses with two probes of at least 300 base pairs and designed from SEQ ID NO:9 (for the 5′ probe) and SEQ ID NO:10 (for the 3′ probe) of the construct (shown in FIG. 2) identified recombinants.

C. Generation of Mutant Mice

Germline mice were generated to create a mouse with a modified IgH locus. ES cell clones showing homologous recombination of Sμ/Sε were injected into C57B6 blastocysts, and the resulting male chimeras were mated with C57B6 females. Germline transmission in heterozygous and homozygous mutant mice was assessed by coat color.

### II. Specified Procedure

A. Target Vector Construction

The construct for targeting the C57BU6 IgE locus in ES cells was made using recombineering and standard molecular cloning techniques. See, for example, Liu et al., A highly efficient recombineering-based method for generating conditional knockout mutations. Genome Research (2003) vol. 13 (3) pp. 476-84.

Targeting constructs were designed based on sequence information available in the NCBI for NT_{—}166318. (See also, Waterston et al., Initial sequencing and comparative analysis of the mouse genome, Nature. (2002) 420(6915):520-62.).

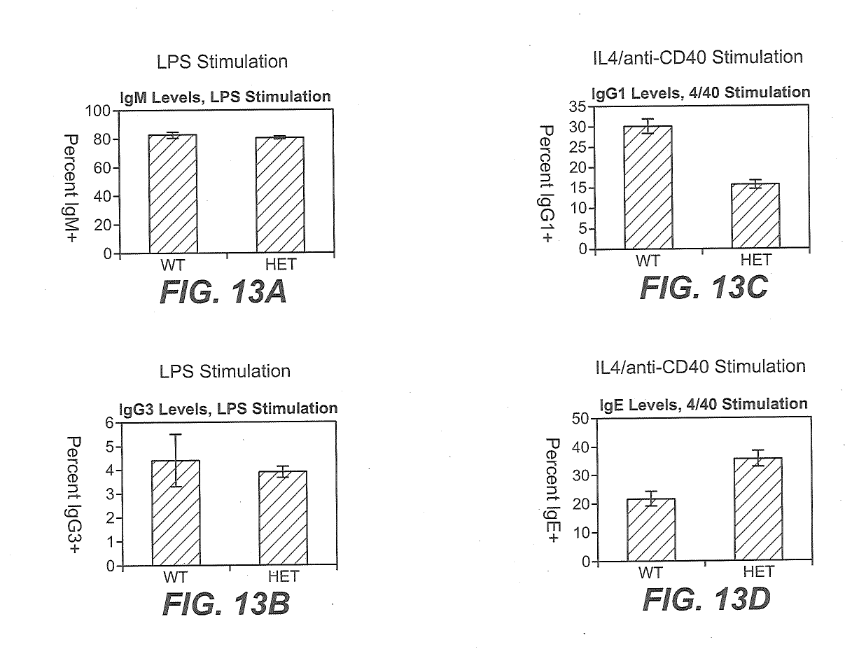

First, a 6988 bp genomic fragment (SEQ ID NO:19; FIG. 13) from a mouse BAC (RP23-135L12; Invitrogen, Carlsbad, Calif.) containing the C57BU6 Switch epsilon (designated as Sε or Sε or Sepsilon, herein) region/sequence was isolated and introduced into a plasmid containing the negative selection marker Diphtheria toxin A (DTA) called “pBlight-DTA” (see Warming et al., Mol. Cell. Biol. (2006) 26 (18):6913-22) for subsequent use in embryonic stem (ES) cell targeting, resulting in “pBlight-DTA-IgE”.

Second, a loxP-PGK-em7-Neo-BGHpAdoxP-HindIII-SalI-AscI-NheI cassette was inserted into the IgE fragment using homologous recombination, replacing the endogenous Sε region with a floxed Neo and a polylinker for subsequent insertion of switch mu region (designated as SMu or Sμ or Sm, herein) (“pBlight-DTA-IgE-lox-Neo-lox-MCS”).

Finally, a 4.9 kb HindIII-NheI fragment containing C57BU6 SMu was isolated from the BAC RP23-135L12 (Invitrogen) and this fragment was cloned into pBlight-DTA-IgE-lox-Neo-lox-MCS using three-way ligation (ligation of a 6.2 kb XhoI-HindIII fragment and a 4.1 kb XhoI-NheI fragment, both from pBlight-DTA-IgE-Neo-MCS, to the 4.9 kb HindIII II-NheI Smu fragment). The resulting construct is called “pSW312” (pBlight-DTA-IgE-lox-neo-lox-Smu). See FIG. 2B.

B. Transformation of Embryonic Stem Cells (ES)

C57BU6 ES cells were targeted using standard methods (G418 positive and DTA negative selection), and positive clones were identified using PCR and taqman analysis. Correctly targeted clones were confirmed by Southern blotting analysis using HindIII digested genomic DNA and an external 3′ probe (the sequence between the 3′ end of the construct and an endogenous IgE HindIII site) (SEQ ID NO:20):

| | |
|---|---|
| taatggacga tcgggagata actgatacac ttgcacaaac tgttctaatc | 50 |
| aaggaggaag gcaaactagc ctctacctgc agtaaactca acatcactga | 100 |
| gcagcaatgg atgtctgaaa gcaccttcac ctgcaaggtc acctcccaag | 150 |
| gcgtagacta tttggcccac actcggagat gcccaggtag gtctacactc | 200 |
| gcctgatgtc cagacctcag agtcctgagg gaaaggcagg ctctcacaca | 250 |
| gcccttcctc cccgacagat catgagccac ggggtgtgat tacctacctg | 300 |
| atcccaccca gccccctgga cctgtatcaa aacggtgctc ccaagctt | 348 |

C. Generation of Mutant Mice

Germline mice were generated to create a mouse with a modified IgH locus. ES cell clones showing homologous recombination of Sμ/Sε were injected into C57B6 blastocysts, and the resulting male chimeras were mated with C57B6 females. Germline transmission in heterozygous and homozygous mutant mice was assessed by coat color.

### Example 2

### In Vitro Stimulation (Anti-CD40/IL4; LPS) to Induce Isotype Switching

The following example details how B-cells were collected and analyzed for class switch recombination (CSR).

Spleen cells from 6-8 week old wild-type or heterozygotes animals were stimulated in vitro with anti-CD40 (1 μg/mL; HM40-3, Pharmingen) plus IL-4 (25 ng/mL), or Lipopolysaccharide (LPS, 20 μg/mL). 1.5×10⁶ cells were seeded in one well of 6 well plates (0.5×10⁶/mL) in RPMI media supplemented with 10% Fetal Bovine Serum, 2 mM glutamine, 100 units/mL of penicillin-streptomycin and 100 μM β-mercaptoethanol. After stimulation, activated B cell cultures were used to generate hybridomas (using standard methods; see, for example, Monoclonal Antibodies: Methods and Protocols in Methods in Molecular Biology (2007) vol. 378:1-13) at day 4-5 or to measure Ig levels by ELISA (day 6). Monoclonal anti-mouse antibodies (Pharmingen) were used to detect Ig levels followed with alkaline phosphotase-conjugated goat anti-mouse IgG1 (Southern Biotechnology) as the detection antibody. Purified mouse Ig (Pharmingen) was used as the standard.

Surface Ig staining was performed using PE anti-mouse Ig (Pharmingen) antibodies on splenocytes. FACS analyses was done at day four of stimulation. Samples were collected on a FACS Scan (Becton Dickinson) and analyzed using Flojo analyses software.

CSR was evaluated by Sothern blot analysis. Briefly, genomic DNA from hybridoma clones was used to assess DNA rearrangement and CSR in IgH locus. Hybridoma genomic DNA (about 150 ul) was digested overnight with EcoRI (NEB) restriction enzyme and the digest was resolved by applying the samples to a 0.7% agarose gel. The resolved samples were then transferred to Zeta-probe blotting membrane (Bio-Rad), fixed by UV cross-linking and/or baking in 80° Celsius vacuum oven (20 min), and probed with ³²P labeled I-mu, C-mu, 1-epsilon, or C-epsilon probes following standard southern blotting protocols (Molecular Cloning, 3^{rd} Edition Vol. 1, pages 6.33-6.64). The labeled DNA was visualized by putting the membranes on X-ray films (Kodak).

In order to sequence the junctions of S-mu and S-epsilon CSR in IgE positive hybridoma clones, nested PCR was used to amplify and sequence this region. First we used I-mu Forward-1:5′-CTCTGGCCCTGCTTATTGTTG-3′ (SEQ ID NO:11) and C-epsilon Reverse-1:5′-CCTGATAGAGGCTGTGAGAAAGGAAGGACC-3′ (SEQ ID NO:12) primers to amplify this region from genomic hybridoma DNA with PCR. The PCR cycles were 94° C. for 2 min; (94° C. for 10 sec, 60° C. for 30 sec, and 68° C. for 150 sec)×35 cycles, 68° C. for 7 min. The product from this PCR step was used as template (2 μl) for a second PCR cycle, using the following primers: E-mu Forward-2: 5′-AGACCTGGGAATGTATGGTT-3′ (SEQ ID NO:13) and C-epsilon Reverse-2: 5′-TAGGTTAGACTTATTTATATCACTGCATGC-3′ (SEQ ID NO:14). The PCR program was the same as above except the annealing temperature was lowered to 55° C. The PCR products were then gel purified (Quigen) and directly sequenced using the following primers: Forward: SM5′: 5′-GTTGAGAGCCCTAGTAAGCG-3′ (SEQ ID NO:15); 9225F: 5′-TTGAGAGCCCTAGTAAGCG-3′ (SEQ ID NO:16); 9518F: TGAGCTCAGCTATGCTACGCGTGTTG-3′ (SEQ ID NO:17); Reverse: 5′-GCCCGATTGGCTCTACCTACCCAGTCTGGC-3′ (SEQ ID NO:18).

### Example 3

### Intracellular IgE Staining & FACS Analysis

This example demonstrates the intracellular staining of IgE and FACS analysis from tissue obtained from heterozygotic mice and wild-type mice after exposure to different stimuli.

0.5×10⁶ splenocytes were spun down and resuspended in FACS buffer (PBS+0.5% Fetal Bovine Serum). Anti-IgE antibody (e-Bioscience, San Diego, Calif.; Cat. No. 14-5992-85) was added at 1 μg/sample to block surface IgE molecules. The cells were incubated for 15 min at 4° C. then washed twice with FACS buffer and pelleted by centrifugation for 3 minutes at 1700 rpm. The pelleted cells were resuspended in 1% Fetal Bovine Serum in PBS.

Cells were vortexed and BD Cytofix/Cytoperm (BD Biosciences, San Jose, Calif.; Cat. No. 554722) was added at 200 μl/sample to fix the cells. Incubated at 4° C. for 20 min.

Cells were washed twice with 1×BD Perm/Wash buffer (BD Biosciences, San Diego, Calif.; Cat. No. 554723) and were resuspended in 250 μl of 1×BD Perm/Wash buffer with Fc-blocker. Cells aliquots were then used for staining with Biotin-Isotype Control (e-Bioscience, 13-4301-82) or anti-IgE-biotin (e-Bioscience, 13-5992-82) along with B220-FITC at 1:200 final antibody dilution. Incubated for 30 min at 4° C.

Cells were washed twice with 1×BD Perm/Wash buffer.

Streptavidin-PE (Pharmingen) in 1×BD Perm/Wash was added at 1:200 dilution. Incubated 10 min at 4° C.

Washed twice with BD Perm/Wash buffer and resuspended cells in 200 μl of FACS buffer and performed FACS analysis using a BD FACS Calibur equipment and CellQuest Pro program to analyze the cells.

Results are shown in FIG. 13. In FIG. 13, it can be seen that the number of cells that stain positive for IgE is increased relative to the WT. FACS results show that the percentage of IgE expressing B-cells increase (approximately twice) in the Het animal compared to WT animal while the levels of IgG1 drops approximately by half in Het compared to WT. This demonstrates that having SmKI (in place of Se) increases switching to IgE and at the same time reducing the chance of switching to IgG1 by competing with this locus.

### Example 4

### IgE Assays/Measurements

This example demonstrates the use of ELISA (luminex or conventional ELISA) to measure the IgE in an unchallenged or challenged recombinant animal or in in vitro cell culture. For in vitro cell cultures, the cells may be stimulated with LPS or antiCD40/IL4.

### Luminex Mouse 7-Plex Immunoglobulin Isotyping Assay

This assay uses a multiplex assay kit (Millipore Beadlyte Mouse Immunoglobulin Isotyping Kit, Cat#48-300) for isotyping (heavy chain: IgG1, IgG2a, IgG2b, IgG3, IgA, IgE, IgM; and light chain: kappa or lambda) mouse monoclonal cell culture supernatants or serum samples (using the Millipore Mouse Isotyping Serum Diluent) in a single well with the Luminex® Instrument system.

Before use, the cell culture supernatants should be centrifuged at 14,000×g to remove any particulates. Similarly, serum and plasma samples should be spun down (8000×g) prior to assay to remove particulate and lipid layers. This will prevent the blocking of wash plate as well as sample needle.

Materials used for Bead-Based Multiplex Assays

Millipore Beadlyte Mouse Immunoglobulin Isotyping Kit Cat#48-300 (Contains: Beadlyte Cytokine Assay Buffer, Cat#43-002, Beadlyte mouse multi-Immunoglobulin Beads, Cat#42-045, Beadlyte mouse Immunoglobulin positive control, Cat#43-008, Beadlyte anti-mouse k light chain, PE (100×), Cat#44-029, Beadlyte anti-mouse lambda light chain, PE (100×), Cat#44-029).

Millipore Beadlyte mouse Isotyping Serum Diluent (5×), Cat#43-033, Phosphate Buffered Saline with 1% Bovine Serum Albumin.

Ig Standard Curve Reagents: Millipore: Beadlyte Mouse Multi-Immunoglobulin Standard (IgG1, IgG2a, IgG2b, IgG3, IgA, IgE, IgM) Lyophilized, from balb/c mouse Cat#47-300.

Filter Plate: Millipore multiscreen-HA 0.45 um surfactant-free.

Millipore Filtration System (NOTE: Any system that provides ddH2O will work.)

The assay may be performed according to the manufacturer's instructions.

### General Protocol for Processing Bead-Based Multiplex Assays

Centrifuge the sample (as appropriate) to precipitate any particulates before diluting into appropriate diluent. Resuspend the standard into appropriate diluent and prepare an eight-point standard curve using twofold serial dilutions. Wet filter plate with 50-100 μl assay diluent per well.

Plate fitting: Add 50 μl of the standard or sample to each well. Sonicate the coupled beads for 15-20 s to yield a homogeneous suspension. Thoroughly vortex the beads for at least 10 s. Dilute the beads to 1500 beads per well, and add 25 μl of diluted bead suspension to each well. Incubate for 15 min in the dark at room temperature (Incubation time can be varied, typically between 15 min and 2 h. The primary incubation of the bead and sample can be performed overnight at 4° C. for greater low-end sensitivity.).

Washing step: Apply vacuum manifold to the bottom of filter plate to remove liquid and blot. Wash by adding 75 μl of assay diluent, vacuum and blot. Repeat washing twice. Resuspend the beads in 75 μl of assay diluent. Add 25 μl of the detection antibody solution to each well. Incubate for 15 min in the dark at room temperature. Apply vacuum manifold to the bottom of filter plate to remove liquid. Wash by adding 125 μl of assay diluent, vacuum and blot. Repeat washing twice. Resuspend the beads in 125 μl of assay buffer. Incubate on a plate shaker for 1 min. Read the results on Luminex® 100 instrument. Data evaluation: extrapolate the sample concentrations from a 4-PL or 5-PL curve.

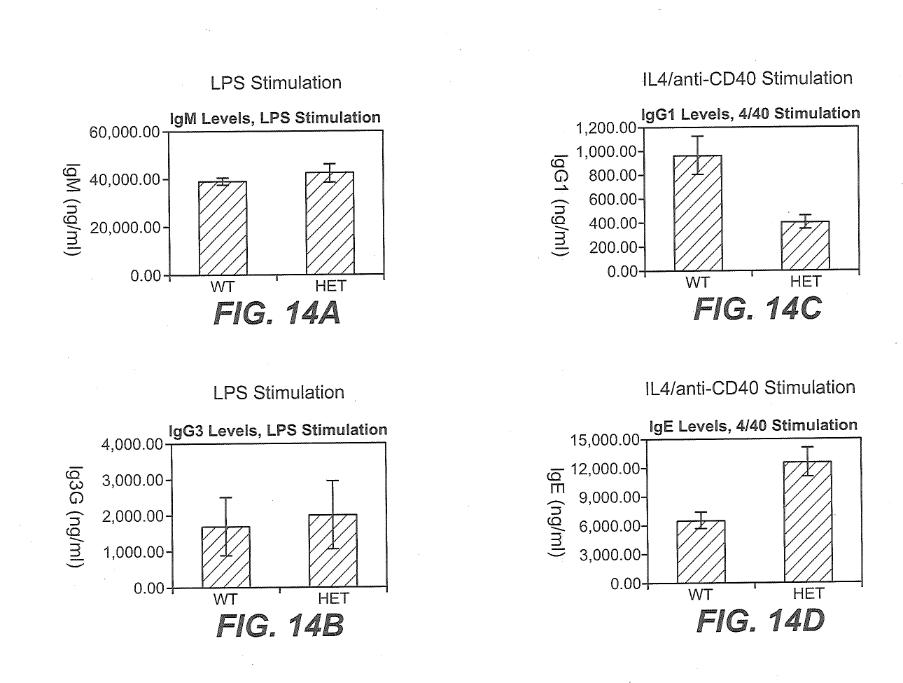

At day 6 post stimulation, supernatants from the same stimulated splenocytes (three Het and three WT mice) that were used for FACS analysis were used in an ELISA assay as described above. In agreement to what we observed in FACS analysis, we also observed an increase in levels of IgE expression and decrease in levels of IgG1 expression in Het compared to WT when stimulated with IL4/anti-CD40. This suggests that there are more frequent breaks occurring in SmKI site that competes with switching to IgG1 and increases levels of IgE switching. LPS stimulation serves as control and shows that both WT and Het have similar levels of IgM and IgG3, suggesting that the locus is intact and functions normally when other switch regions are accessible for class switching. See FIG. 14.

### Total Mouse IgE Binding ELISA

This assay is run to quantitate mouse IgE serum levels in both naïve and immunized animals.

I. Coat with Capture Antibody:

Dilute the purified anti-mouse IgE capture mAb (Rat Anti-mu IgE, Clone R35-92 BD Pharmingen, San Diego, Calif., Stored at 4° C. Cat. #553416 (0.5 mg/ml)) to 2 μg/ml^{a} in coating buffer (0.05 M Carbonate/bicarbonate, pH 9.6. Add 100 μl per well to an enhanced protein-binding ELISA plate (e.g., Nunc immunoplate Cat #464718, 384-Well). Shake plate to ensure all wells are covered by capture antibody solution. Cover the plate and incubate for overnight at 4° C. [Note: may be done at 1 hour at 37° C. Wash the plate 3× with PBS/Tween® (PBS+0.05% Tween 20). For each wash, wells are filled with 200 μl PBS/Tween® and allowed to stand at least 1 min prior to aspirating or dumping. As a final step, tap plate on paper towels to remove excess buffer.

### II. Blocking:

Block the plate with 50 μl blocking buffer (PBS+0.5% BSA+10 ppm Proclin pH 7.4) per well. Cover the plate and incubate at RT for 1 hour with gentle agitation. Wash the plate 3× with PBS/Tween®, as above.

### III. Apply Standards and Samples:

Leave column 1 as blank wells (i.e., no antigen added, 25 μl per well blocking buffer only). Use columns 2 and 3 for duplicates of the standard, 25 μl per well: Prepare standard curve from 500 ug/ml Stock standard antibodies to starting standard concentration of 10 ng/ml (1:50,000). Make Serial 1:2 dilutions (PBS+0.5% BSA+0.05% Tween 20+15 ppm Proclin+0.2% BgG+0.25% CHAPS+5 mM EDTA, pH 7.4) The mouse IgE standard curve: 10.0, 5.0, 2.50, 1.25, 0.625, 0.313, 0.156, 0 ng/ml. Assay Controls are mouse IgE κlsotype control, BD Bioscience, Catalog #557079, Main Stock: 0.5 mg/ml, keep in 4° C.) at the following dilutions: 8 ng/ml, 4 ng/ml, 0.5 ng/ml. Use the remaining columns to add samples at various dilutions in blocking buffer, 25 μl per well. Dilute the serum samples with Assay Diluent (PBS+0.5% BSA+0.05% Tween 20+15 ppm Proclin) 1:25 minimum initial dilution, serial 1:3, using Hamilton Diluter. Cover the plate and incubate for 2 hours with gentle agitation. [Note: May be done for at least 1 hour at RT or overnight at 4° C.] Wash the plate 6× with PBS/Tween®, as above, and incubate with agitation for one hour.

IV. Incubation with Detection Antibody:

Add 25 μl biotinylated anti-mouse IgE (Rat Anti-mu IgE-Biotin, Clone R35-118, BD Pharmingen, San Diego, Calif., 0.5 ug/ml, 4° C., Cat #553419) per well. Cover the plate and incubate at RT for 30 minutes with gentle agitation. Wash the plate 6× with PBS/Tween®, as above.

### V. Add Streptavidin-Horseradish Peroxidase (SAv-HRP):

Dilute Streptavidin-HRP (GE Healthcare, formerly Amersham Biosciences, Piscataway, N.J., 1 mg/ml, Stored at 4° C., Cat.#RPN4401) 1:20,000 in blocking buffer to a final concentration of 50 ng/ml. Add 25 μl per well and incubate with agitation for 30 minutes. [Note: Avidin-HRP may be used instead of Streptavidin-HRP with appropriate modification as noted in the art.] Cover the plate and incubate at RT for 30 min. Wash the plate 6× with PBS/Tween®, as above, of this protocol.

### VI. Add Substrate and Develop:

Mix 1 Part of TMB A to 1 Part of TMB B (TMB peroxidase solutions A & B (KPL, Gaithersburg, Md., cat #50-76-02 and 50-65-02, respectively), store at 4° C.). Add 25 μl TMB substrate to each well and shake. Incubate 15 min. at room temperature for color to develop and add 25 μl of 1 M H₃PO₄ to quench the development. Read the plate at 450-650 nm.

Interpolate the serum sample IgE levels from the standard curve.

### Example 5

### Hybridoma to Quantitate Antibody Isotypes

Hybridomas are constructed using techniques known in the art. Using the assays of Example 4 the immunoglobulin isotypes are characterized.

The antibodies are then further characterized for binding affinity, epitope characterization and mode of action on a relevant pathway.

### Example 6

### Immunization: TNP-OVA; OVA; Ficol to Induce Isotype Switching In Vivo

This example illustrates the in vivo immunization of recombinant animals as described herein.

Eight-week-old sex-matched Balb/C mice aged 8 weeks, weighing approximately 25-30 g are immunized i.p. with TNP-OVA 50 ug/alum 2 mg or TNP-Ficoll 50 ug injected i.p. in 100 ul sterile PBS, boosted at day 28. 60 ul samples are collected on day −3, 7, 14, 21, 28, and 42 via tail vein for antibody isotype measurements using the assays described in Example 4.

II. Antigen-Induced Peritonitis Model with Measurement of Histamine in the Peritoneal Fluid.

### Animals and Sensitization Procedure

Balb/C mice aged 8 weeks, weighing approximately 25-30 g raised at the Pasteur Institute (Paris, France), are actively sensitized by a subcutaneous (s.c.) injection of 0.4 ml 0.9% w/v NaCl (saline) containing 100 gLg ovalbumin adsorbed in 1.6 mg aluminium hydroxide (Andersson & Brattsand, 1982). Seven days later, the animals receive the same dose of ovalbumin in the presence of Al(OH)₃ and are used 7 days thereafter.

### Antigen-Induced Peritonitis

Peritonitis is induced by the intraperitoneal (i.p.) injection of 0.4 ml of a solution containing 2.5 or 25 gm/ml ovalbumin diluted in sterile saline (1 or 10 μg of ovalbumin, as final doses injected per cavity). Control animals receive the same volume of sterile saline. At various time intervals after antigen challenge (30 min-164 h), animals are euthanized by an overdose of ether and the peritoneal cavity is opened and washed with 3 ml of heparinised saline (10 Upper ml). Approximately 90% of the initial volume is recovered. In rare cases, when hemorrhages are noted in the peritoneal cavity, the animals are used.

Histamine levels are measured using methods known in the art.

### Example 7

### Infection with Nippostrongylus brasiliensis to Induce IgE

This example illustrates the IgE response to infection with parasitic worms, Nippostrongylus brasiliensis.

The course of development of N. brasiliensis has been well characterized in the mouse (Love, Nippostrongylus brasiliensis infections in mice: the immunological basis of worm expulstion, (1975) Parasitiology 70:11). In the mouse once infective larvae (L3) have penetrated the skin they are transported to the lungs via the lymph and blood vascular systems. After a tracheal-esophageal migration, fourth-stage larvae (approx. 15-35% of the original L3 dose) are carried to the lumen of the small intestine and mature. The infection is patent by the seventh day after larval inoculation. Worm expulsion that is precede by a sharp fall in fecal egg output occurs soon after patency (approx. day 9 and is virtually complete by the twelfth day.

The maintenance of N. brasiliensis under laboratory conditions, methods of infection, worm transfer and collection of worms for counting have been described previously (Love & Ogilvie, Nippostrongylus brasiliensis in young rats. Lymphocytes expel larval infections but not adult worms. (1975) Clin. Exp. Immunol. 21:155). Live worms are purified from a worm mount. The purified worms are counted and resuspended at 2500 worms/ml in phosphate buffered saline (PBS). Mice are infected with 500 worms/200 μl via a subcutaneous injection as described by Ogilvie (Reagin-like antibodies in animals immune to helminth parasites Nature (1964) 204:91). The infected mice are kept on a normal diet and provided ad lib antibiotic water (0.5 g polymyxin B and 10 g neomycin sulfate in 5000 ml ddH₂O) for 5 days. The mice are checked for lung inflammation at day 9 and serum IgE levels checked at days 9 and 15 using the methods provided in Example 4.

### Example 8

### Sensitization with Panel of Allergens to Induce IgE (Airways, Skin)

This example illustrates the IgE response to various allergens.

A panel of allergens (used in clinic) such as Dust mite D. farinae, D. pteronyssinus, American Cockroach, Alternaria tenuis, Aspergillus mix, Cladosporidium herbarum, Cladosporidium herbarum, Cat, Dog, Plantain-Sorrel mix, Short Ragweed, West Oak mix, Grass mix/Bermuda/Johnsonand fungus and other allergens are injected with varying doses and the serum immunoglobulin levels are assessed as described above.

### Example 9

### Evaluation of Serum IgE or Memory IgE Positive B Cells Following Administration of Desired Therapeutics

This example illustrates how various therapeutics influence the IgE response under various conditions. Both preventative and therapeutic interventions are evaluated in a similar manner. Reference to proposed therapeutic agents is intended to cover both preventative and therapeutic interventions.

The serum IgE concentration of naïve animals is measured. The animals are randomly assigned to one of seven group. The first group will receive no therapeutic intervention or antigen challenge. The second group receives vehicle only (i.e., no antigen) then the proposed therapeutic agent. The third group receives antigen challenge then the proposed therapeutic agent. The fourth group receives the proposed therapeutic agent then vehicle only. The fifth group receives the proposed therapeutic agent then antigen challenge. The sixth group group receives vehicle only (i.e., no proposed therapeutic agent). The seventh group receives antigen challenge only (i.e., no proposed therapeutic agent).

The time between antigen challenge and the administration of the proposed therapeutic agent (or vice versa) may be varied to determine optimal administration times.

The animals IgE levels are measured over time to evaluate the proposed therapeutics ability to modulate IgE serum levels. 60 ul samples are collected on days 3, 7, 14, 21, 28, 35 and 42 (post antigenic challenge) via tail vein for antibody isotype measurements using the assays described in Example 4.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### INDUSTRIAL APPLICABILITY

The embryonic stem cells provided herein allow the generation of an in vivo model IgE response to non-specific allergens.

The in vivo animal model described herein provides a full repertoire IgE response to a non-specific allergen.

TABLE 1
Summary of Sequences

| SEQ ID | | |
|---|---|---|
| NO | Sequence | Figure |
| 1 | GGGCTGGGCTG | N/A |
| 2 | GAGCTGAGCT | N/A |
| 3 | tgggttaagc agagctgtgc tgggctggta tgagctggtc | 3 |
| | caagttgggc taaacagagc tgggccaggc tagtatgagc | |
| | tggtctgaac tacactaagc aggactaggc tgggctgagc | |
| | tgagctggac tggctggact tggctgagat gtgttgagct | |
| | gggttaagta tggctgggct gggctggcct gggctgggct | |
| | ggactggatt ggtatgagct ggtccaagtt gggctaagca | |
| | gagctgggcc aggctggtat gagctggtct aaactgaact | |
| | aagtagggct gggctaagct gagctggtct acactagcct | |
| | gacctgagct agggtaggct ggactgggct gagctaagtt | |
| | gcactgggca ggggtgggct ggaccgagct gatttgagct | |
| | gggatgggct gagatgggtt cagcaggcct aagcaggcct | |
| | agctgggttt agctagattt agctaggcaa ggctgagcta | |
| | ggctgggcgg ggcggggcta ggctgggcag ggctggactg | |
| | agctagcttt tgtatattcg gttgaaatgg gttggtctgg | |
| | tctggactga actgactgag ctgggctagc ctgagctcga | |
| | tggggggtat actcagctga gatgggctgg tctggctaga | |
| | ctgaactgga ttgggctagg ctgagctagg ctgacctgaa | |
| | ctggcctggt ctgggctgga ctgggcaggg ctggtctcag | |
| | ctagactaca ctgagttaac ctgggctgga ccatactggg | |
| | ttaaactagg ttgcactggc tgggttagac ttggctgagc | |
| | tgggcttggc tgagctgagt caagatggtc tgagttgatt | |
| | tgagttggct aagctaagct gagctacact gaactaggca | |
| | aggctgggct ggaaaggtct gggttaagtt aggagggact | |
| | tggcttggct tagctgggcc aagctaggct gaactgggct | |
| | gaactgagct gagctgggct gagctgggct gagctgggct | |
| | gagctgggca aggctaaact ggaatggact gaattggcct | |
| | aagatgggcc cggctaagct aagtaaggct gccctgaact | |
| | gagcaggact ggcctggcct ggattgacct ggcatgagct | |
| | taacttgact agactagtct atcttgggtg aactgggcta | |
| | agcaggacta atctggcctg atctgagcta gactgaacta | |
| | ggctaagctg agctgagttt agcttggctg aactgggctg | |
| | ggctgcactg aactgtattg agctatgtag aactgagctg | |
| | gtcttgtctg aggtgggttg ggctggtctg ggctgaacca | |
| | gattgcacta gactgagctt agctggacct ggctgagctg | |
| | gactgcattg tgctaaactg gctctcttta gaccgagctt | |
| | agctggactg gactgagcta ggttgggtgg gctgatctaa | |
| | gctgagctag gctggtctca cctgaggaat gctgtgctgt | |
| | gctgagctga actaaactga gctcagctaa ggaagtgtga | |
| | gctagactga gctgagctag gctgggttgg gctgaactga | |
| | gctaccttgg gtggactagg ctgagctgag ctgggttgag | |
| | ctgagctata gatttggttg gactggactg gattgggcta | |
| | aactgaactg gtttggggta ggctgggatg agctggactg | |
| | agctaggctg tactggtctg agctaaacta gaatgagcta | |
| | ggctagggtt gtgagctagg gttgtactgg tctaagctga | |
| | gtttagctga gagaggctgg gctagacttc cataaggtgg | |
| | ctgagtcata ctacagtgca ctgagctgtg ttgagcttaa | |
| | cttggattaa gtggaatggg ttgagctggc tgaactgggc | |
| | tgaactgaga taaactagac tgagctggga cacgctggga | |
| | cgagctggaa cgagctagaa ttactgttct aatctgatct | |
| | gggctgaggt aaactgggcc tggttgagct ctactaggct | |
| | aagtagagtt gagct | |
| 4 | AAGGCTAGGT GGGGCAAGAC GAGGGAACTA AAGCCACTGT | 7A |
| | GCTGCTGGGG ACACTGTGGA CACtgatgga cagaaaggga | |
| | gtgatcagtc tgtggacagg agggggaggg gCAAGGATGA | |
| | TGCTGACAGA GAGTCACAGT GGAGTCCGTA GCAGGAAAGA | |
| | GAGAGAGCGC CCAGTGTAGT CCTAAGGCTT AGGAAGTTGC | |
| | AACTGCCTCC TCTCCTTCCA GAGGATCACT CACTGCCACC | |
| | TAGCATAGAA CTCAGAGGAC CCAGAACCAG CAGCTCAGCC | |
| | CAACCTGTGT GTCACAGAAG AATCAGGCCC GGTCAGGCTA | |
| | GACACAAAGG CTCTTGGCCC TCATGCTGTG AGGGAGGTAC | |
| | ACACTGGAGG CACACCACAA ACAGTTGGAG CAGAGGCTTC | |
| | TCGCCCCTAT TTTTCCCTCT GAACAATAGT TGCTTCCAGG | |
| | GAACTCTGCA TTTACCCCTC AGGCTCCCAC CCATGTCTAT | |
| | TAGGCTGAAG GCCAAGCCTG TCACCTCAGA CAGACAGTGT | |
| | ATCTGAAAGA CAGAAGGCCG TGCAAGACCA CAATTCCCTT | |
| | GAATCTCACA CTCTGTCTTC CCAAAGTTCC TAACTGCATC | |
| | TGACCTTTCT GGGCCAGCCT CTCAGCCTGC CTGGCTCTGC | |
| | CACTATCAGG AAGATCTCTA ATATCTTCCA AATGCAATTA | |
| | AACACGCTCC TGTGAAAGTC AGACTTGGCA TAGCCTAAGT | |
| | CCCTTCGGTC CCTTTCACTG GGACCAACGA CCCTGAGCAG | |
| | CCAGGGTCCA AGGGATGGGG CTCTCATTTT CTTCCCCAAA | |
| | TCTCTGTGTG CCTCTCTCAA GACTCAAGAC TCACAAGCAA | |
| | AATTAGTGGC TCCTATAGTT TGTATGTATG TTTTCTTAGA | |
| | ACTCCTAGGA ACCATGGGCC TACAGAGACA TCAGAGTGTA | |
| | GAGGGAATCC CTGAACCCAG AAGATGACCT TGCTCTACAA | |
| | AGCTGCAGCT GAGACAGACA CTACTAGTAC CCCATGAAAG | |
| | CTGCTGAGCC AAAGCCCAGC CCTCACACCA TCTTTACCCT | |
| | CATCCCTCCC CTCAGTGCAG ACATAGACCA CAGGCCTGGA | |
| | AGAGACGTTA GCTGTTTCTA CACAGCTCCG TGAAACCCAG | |
| | TCACAACCCA GATGTGCTCT GTCCTTCTGG ACTCCTTGCC | |
| | AGAGTAGCAG GTAGAGGACC TCAAGCTGAA AGATAATCAC | |
| | TTGTGAGTGG GCACCAGGGA AGGCCACTGT CCCTCGCATG | |
| | CCAGCTCCAA AGCTGATACA GGAACTAGGG TGCCTCTATC | |
| | AGAGGCCCTG CAATGTCATA TCTGGCCCAC AGGCTGTTCC | |
| | TCTTTGTGCA CCATTAATAA CTTACAAAGT GACAGCCACA | |
| | CTCCCCTGAA GGGCTGCCAA AGGAACAGAA AAAGCAATGG | |
| | CGAGGGTCTA GTCCTGCCTC AGGGCAGTGA CACTCCAAAG | |
| | GGGCAGGCAT GGTGACTGCA CGCASNNCAC ACATGCAAGG | |
| | CTTTAATACG AGAGCTATGC AAGGAGACCT GGGATCAGAC | |
| | GATGGAGAAT AGAGAGCCTT GACCAGAGTG TGCAGGTGTG | |
| | TCTCCTAGAA AGAGGCCTCA CCTGAGACCC CACTGTGCCT | |
| | TAGTCAACTT CCCAAGAACA GAATCAAAAG GGAACTTCCA | |
| | AGGCTGCTAA GGCCGGGGGT TCCCACCCCA CTTTTAGCTG | |
| | AGGGCACTGA GGCAGAGCGG CCCCTAGGTA CTACCATCTG | |
| | GGCATGAATT AATGGTTACT AGAGATTCAC AACGCCTGGG | |
| | AGCCTGCACA GGGGGCAGAA GATGGCTTCG AATAAGAACA | |
| | GTCTGGCCAG CCACTCACTT ATCAGAGGAC CTCAGGTATT | |
| | ACAACCCATG GGACCCTGAG CAAAAGGGTT TGCCTAAGGA | |
| | GAAGGGACAA ACAGGTTACA GGGTCCTGGG TGGGGAAGGG | |
| | GACACCTGGG CTGCCTTCTA ATGTGGACAG TCTCTTGACC | |
| | ACCGAATGTC CTTCAGCTAT CACTTCCCTG CACTAAGGCA | |
| | CACAGGTATT AGAAACTGCT ATAGCTATTC ATGAAGACGG | |
| | GGGACTGTGG ATCTCAACCA GAGAGGGCTG AACCAAGATA | |
| | AACTGAATAT GTTGTGAGAA ACTCAAAAAC TGCAGGAGAG | |
| | GCTGGAGAGG AATCGGCCAG CAAGCCATCA GACAAGAATG | |
| | CAATGACAAA TGTCAGATCC AGATAATCAC AGCAAGGAAT | |
| | TGCCCTGTGA TGAACTAACA ACCAAGAGGA CTGTCCACAG | |
| | CTGGGCTGAC CCAGGCAGCA CTGGGCTAAA TTGGGTGGGA | |
| | TCTGTGCTGC CCTGGGCTGG TATGAGCCAG GATGAGCCAA | |
| | GTGAAGTGGG CTGGACTAGT TTGGGCTGGA CTGGCCTGGA | |
| | GTAGGCTAAA CCAGTTTAAA CTAGAGTAGG CTGGGCTGAG | |
| | GTGAATCAGA CTAGGCTAGA CTAGTCTGAG C | |
| 5 | GATCCCTGTG AAGCCCTGGG CCATGGGAAG AGATAGAAGG | 7B |
| | AAGGCTAGGT GGGGCAAGAC GAGGGAACTA AAGCCACTGT | |
| | GCTGCTGGGG ACACTGTGGA CACTGATGGA CAGAAAGGGA | |
| | GTGATCAGTC TGTGGACAGG AGGGGGAGGG GCAAGGATGA | |
| | TGCTGACAGA GAGTCACAGT GGAGTCCGTA GCAGGAAAGA | |
| | GAGAGAGCGC CCAGTGTAGT CCTAAGGCTT AGGAAGTTGC | |
| | AACTGCCTCC TCTCCTTCCA GAGGATCACT CACTGCCATC | |
| | TAGCATAGAA CTCAGATGAC CCAGAACCAG CAGCTCAGCC | |
| | CAACCTGTGT GTCACAGAAG AATCAGGCCC AGTCAGGCTA | |
| | GACACAAAGG CTCTTGGCCC TCATGCTGTG AGGGAGGTAC | |
| | ACACTGGGGG CACACCACAA ACAGTTGGAG CAGAGGCTTC | |
| | TCACCCCTAT TTTTCCCTCT GAACAATAGT TGCTTCCAGG | |
| | GAACTCTGCA TTTACCCCTC AGGCTCCCAC CCATGTCTGT | |
| | TAGGCTGAAG GCCAAGCCTG TCACCTCAGA CAGACAGTGG | |
| | ATCTGAAAGA CAGAAGGCCG TGCAAGACCA CAATTCCCTT | |
| | GAATCTCACA CTCTGTCTTC CCAAAGTTCC TAACTGCATC | |
| | TGACCTTTCT GGGCCAGCCT CTCAGCCTGC CTGGCTCTGC | |
| | CACTATCAGG AAGATCTCTA ATATCTTCCA AATGCAATTA | |
| | AACACGCTCC TGTGAAAGTC AGACTTGGCA AAGCCTAAGT | |
| | CCCTTCGGTC CCTTTCAGTG GGACCAACGA CTCTGAGCAG | |
| | CCAGGGTCCA AGGGATGGGG CTCTCATTTT CTTCCCCAAA | |
| | TCTCTGTGTG CCTCTCTCAA GACTCAAGAC TCACAAGCAA | |
| | AATTAGTGGC TCCTATAGCT TGTATGTATG TTTTCTTGGA | |
| | ACTCCTAGGA ACCATGGGCC TACAGAGACA TCAGAGTGTA | |
| | GAGGGAATCC CTGAACCCAG AAGATGACCT TGCTCTACAA | |
| | AGCTGCAGCT GAGACAGACA CTACTAGTAC CCCATGAAAG | |
| | CTGCTGAGCC AAAGCCCAGC CCTCACACCA TCTTTACCCT | |
| | CATCCCTCCC CTCAGTGCAG ACATAGACCA CAGGCCTGGA | |
| | AGAGACGTTA GCTGTTTCTA CACAGCTCCG TGAAACCCAG | |
| | TCACAACCCA GATGCGCTCT GTCCTTCTGG ACTCCTTGCC | |
| | AGAGTAGCAG GTAGAGGACC TCAAGCTGAA AGATAATCAC | |
| | TTGTGAGTGG GCACCAGGGA AGGCCACTGT CCCTCGCATG | |
| | CCAGCTCCAA AGCTGATACA GGAACTAGGG TGCCTCTATC | |
| | AGAGGCCCTG CAATGTCATA TCTGGCCCAC AGGCTGTTCC | |
| | TCTTTGTGCA CCATTAATAA CTTACAAAGT GACAGCCACA | |
| | CTCCCCTGAA GGGCTGCCAA AGGAACAGAA AAAGCAATGG | |
| | CTAGGGTCTA GTCCTGCTTC AGGGCAGTGA CACTCCAAAG | |
| | GGGCAGGCAT GGTGACTGCA CACA---CGC ACATGCAAGG | |
| | CTTTAATACG AGAGCTATGC AAGGAGACCT GGGATCAGAC | |
| | GATGGAGAAT AGAGAGCCTT GACCAGAGTG TGCAGGTGTG | |
| | TCTCCTAGAA AGAGGCCTCA CCTGAGACCC CACTGTGCCT | |
| | TAGTCAACTT CCCAAGAACA GAATCAAAAG GGAACTTCCA | |
| | AGGCTGCTAA GGCCGGGGGT TCCCACCCCA CTTTTAGCTG | |
| | AGGGCACTGA GGCAGAGCGG CCCCTAGGTA CTACCATCTG | |
| | GGCATGAATT AATGGTTACT AGAGATTCAC AACGCCTGGG | |
| | AGCCTGCACA GGGGGCAGAA GATGGCTTCG AATAAGAACA | |
| | GTCTGGCCAG CCACTCACTT ATCAGAGGAC CTCAGGTATT | |
| | ACAACCCATG GGACCCTGAG CAAAAGGGTT TGCCTAAGGA | |
| | GAAGGGACAA ACAGGTTACA GGGTCCTGGG TGGGGAAGGG | |
| | GACACCTGGG CTGCCTTCTA ATGTGGACAG TCTCTTGGTC | |
| | ACCGAATGTC CTTCAGCTAT CACTTCCCTG CACTAAGGCA | |
| | CACAGGTATT AGAAACTGCT ATAGCTATCC ATGAAGACAG | |
| | GGGACTGTGG ATCTCAACCA GAGAGGGCTG AACCAAGATA | |
| | AACTGAATAT GTTGTGAGAA ACTCAAAAAC TGCAGGAGAG | |
| | GCTGGAGAGG AATCGGCCAG CAAGCCATCA GACAAAAATG | |
| | CAATGACAAA TGTCAGATCC AGCAAATGAC AGCAAGGAAT | |
| | TGCCCTGTGA TGAACTAACA ACCAAGAGGA CTGTCCACAG | |
| | CTGGGCTGAC CCAGGCAGCA CTGGGCTAAA TTGGGTGGGA | |
| | TCTGTGCTGC CCTGGGCTGG TATGAGCCAG GATGAGCCAA | |
| | GTGAAGTGGG CTGGACTAGT TTGGGCTGGA CTGGCCTGGA | |
| | GTAGGCTAAA CCAGTTTAAA CTAGAGTAGG CTGGGCTGAG | |
| | GTGAATCAGA CTAGGCTAGA CTAGTCTGAG C | |
| 6 | agctcacccc agctcagctc AGCTCACCCC AGCTCAGCCC | 8 |
| | AGCTCAGCCC AGCTCAGCCC AGCTCAGCCC AGCTCAGCCC | |
| | AGCTCAGCTC AGCTCAGCCC AGCTCAGCCC AGCTCACCCC | |
| | AGCTCAGCTC AGCTCACCCC AGCTCAGCCC TGCTCAGCCC | |
| | AGCTCAGCTC ACCCCAGCTC AGCTCAGCTC AGCTCACCCC | |
| | AGCTCAGCTC AGCTCACCCC AGCTCAGCTC AGCTCAGCCC | |
| | AGCTCAGCTC AGCTCACCCC AGCTCAGCTC ACCCCAGCTC | |
| | AGCCCAGCTC AGCCCAGCTC AGCCCAGCTC ACCCCAGCTC | |
| | AGCCCAGCTC AGCTCAGCTC ACCCCAGCTC AGCTCAGCTC | |
| | AGCTCAGCTC AGCTCAGCTC AGCTCAGCTC AGCTCAGCTC | |
| | AGCTCAGCTC AGCTCAGCTC AGCTCAGCTC AGCCCAGCTC | |
| | ACCCCAGCTC AGCTCACCCC AGCTCAGCTC AGCTCACCCC | |
| | AGCTCAGCTC AGCTCAGCTC AGCTCACCCC AGCTCAGCCC | |
| | AGCTCACCCC AGCTCAGCCC AGCTCAGCTC ACTCCAGCTC | |
| | AGCTCAGCTC ACCCCAGCTC ACCCCAGCTC AGCTCAGCTC | |
| | AGCTCACCCC AGCTCAGCTC AGCTCAGCTC AGCTCAGCTC | |
| | ACCCCAGCTC AGCTCAGCTC ACCCCAGCTC AGCTCACCCC | |
| | AGCTCAGCTC AGCTCACCCC AGCTCAGCTC ACCCCAGCTC | |
| | ACCCCAGCTC AGCTCAGCTC ACCCCAGCTC AGCTCAGCTC | |
| | ACCCCAGCTC AGCTCACCCC AGCTCAGCCC AGCTCAGCCC | |
| | AGCTCAGCCC AGCTCACCCC AGCTCAGCCC AGCTCAGCTC | |
| | ACTCCAGCTC AGCTCAGCTC ACCCCAGCTC ATCCCAGCTT | |
| | ACCCCAGCTC AGCTCAGCTC ACCCCAGCTC AGCCCAGCTC | |
| | ACCCTAGCTC AGCCAAGCTC AGCTCAGCTC ACCCCAGCTT | |
| | AGCTCAGCTC ACCCAGCTCA GCTCACCCTA GCTCAGCTCA | |
| | GTCTAGCTCA GCCCAGCTCA GCTCACCCCA TCTCACCCCA | |
| | TCTCAGCTAC TCCAGAGTAT CTCATTTCAG ATCAGCTCAC | |
| | CCCAACACAG CGTAGCATAG CTGAGCTCAC CCCAGCTCAT | |
| | CTCAGCTCAG AACAGTCCAG TGTAGGCAGT AGAGTTTAGC | |
| | TCTATTCAAC CTAGATTAAT GAAGTTCATT CCAGTTTGGC | |
| | TCATCTCGGT TAAGCCAGCC TAGTTTAGCT TAGCGGCCCA | |
| | GCTCATTCCA GTTCATTACA GTCTACTTCA TTTTGGCTCA | |
| | AGCCCAGCTT TGCTTACCTC AGAGTAATCA CCTCAGTTTA | |
| | GCGCATTTTA GAAGCACTCA GAGAAGCCCA CCCATCTCAG | |
| | CTCAGCTGTG CTTTTTAGAG CCTCGCTTAC TAGGGCTCTC | |
| | AACCTTGTTC CCTTAATTTT GCTCAGCAAG CTTTATGAGT | |
| 7 | TATGAGATGA AAGTAAGCTG AGTTGGGCAG TTCTAGACTA | 9A |
| | TTCTAGGCTG TTCTGGGCTG ACCTGAACTG GGTGGGGTTG | |
| | AGCTGAATGA AGTAGGCTGT GCTGAGCTCT GCCAGGCTGG | |
| | ATGAGTTTAT TGATCTGAGT TGGACTGGCC TGGGCTGCAC | |
| | TAAATGGGAC TGAGATGAGA TTGGCCAGGC TAGGAGGGAT | |
| | TGAGCAAGGC TAAGCTAAGT TGTTCTGGAC TAGGCCAAAC | |
| | TATTAGAGGT CTTTTGGTTT AGTTGAACTT TCTGGTACTG | |
| | AACTAAACTG TCTTTAAGAT AGAATGCTCA AAATTATTTG | |
| | TGGGTGTTTT AACTGTCCTC AAAGAAGATT GTCCTGTTGT | |
| | AGGATACAAC AACAGCTACT AGCCAGACTG GGTAGGTAGA | |
| | GCCAATCGGG CCTAGCAGGA ATATCCTGTG CTTTCTGAGG | |
| | ACCTGGCACA GAGCTGAGCT GAGCCCCTCT CTCAGGAGAA | |
| | TGTCCTGGGC ATGTGGACAC TCTAGAGCAT CAAGGTGGCT | |
| | TCTGAAGTGG TTGTATTCTC TATGTGCTTT CTGGGATCCA | |
| | CGGAGGTCAT CTTGGAGGCA GAACACTGTG CAGGTTAGCC | |
| | TATGGTAAAG CAGAGAGCCT CATGTATCTG AAACCCAAAG | |
| | ATCCGATTAA TTGCCATTGT AAGTTTGCCT CTTCATCCAA | |
| | ACTCGTGCCC AGCTCTCCTG GAAGCCCCTG TGCTAAGCCA | |
| | GCTAGGGGCA GTGAGTGAGC AAAGCCTGAT GGGGTGTAAG | |
| | GAATCAGGGG GATCCCTAGG TCTGTGTTTG GGTTTAGTGA | |
| | ATAAAGACAA GACCCAGAAG GAATCTATGA CCAACAGCCC | |
| | TAGGAAACAA GAATCTCACC ATTCTGTCCT CAATGTGTCC | |
| | CAAAACAGAT TTAATGTGTC TCACCAAGAA ACTGGTGGTC | |
| | CTGGGAAAGC TCTGAATCCC CAGGCCCCAA GAGTGGGGAC | |
| | AGAAGAGACA ATGGCAATTC ATCGGATCTC TGGGCCACCA | |
| | AGCCCTGTGG GGTACCTATG TCCTGGACAT AAAGGACAAC | |
| | CTAGTCCCTC TGTCAACATT ACATAGCCTA CCTTAAAGCT | |
| | ACTCCATTCA TCCTGAGACC ATAATGGCTT CCAGTCTGCC | |
| | ACCCAGCTCT CATGCTTCAT TTCTGGACAT TCCCTAGATG | |
| | GCGTCACTGT CACCTGGTCT AAAGGACAGA CAGGAGATAC | |
| | CTCACACATA TCCACAAAAT TTCCCAATCA AGAAAGAGGG | |
| | CAAGTTTGCC TCTTTATCCA AACTTGTGCC CAGCTCTCCT | |
| | GGAAGCCCCT GTGCTAAGCC AGCGAGGGGC AGTGAGTGAG | |
| | CAAAGCCTGG CGGGGTGTAA GGAATCAGGG AGCTCCCTAG | |
| | GTCTGTGTTT GGGTTTAGAG AATAAAGACA AGACCCAGAA | |
| | GGAATCTAAC CATCTGTCTC CTAGACTGGA ATGGGGTCCC | |
| | CAGAGCCCTG CTCCTGTCAC AGCTGCCCTT AATCAGTTCC | |
| | CCATGCTGCA GSNNGCATGC AGTGATATAA ATAAGTCTAA | |
| | CCTAGGTCCT TCCTTTCTCA CAGCCTCTAT CAGGAACCCT | |
| | CAGCTCTACC CCTTGAAGCC CTGTAAAGGC ACTGCTTCCA | |
| | TGACCCTGGG CTGCCTGGTA AAGGACTACT TCCCTGGTCC | |
| | TGTGACTGTG ACCTGGTATT CAGACTCCCT GAACATGAGC | |
| | ACTGTGAACT TCCCTGCCCT CGGTTCTGAA CTCAAGGTCA | |
| | CCACCAGCCA AGTGACCAGC TGGGGCAAGT CAGCCAAGAA | |
| | CTTCACATGC CACGTGACAC ATCCTCCATC ATTCAACGAA | |
| | AGTAGGACTA TCCTAGGTAA GTAGGGATGG GCTGacagtt | |
| | acactgtgta ttctcccttg gagatggaac agtttctgtc | |
| | taatcaggaa cttgtcacaa tttcctttca tagaggactt | |
| | cataagagat ttttttt-ct acttctatca tgtttagtga | |
| | tccaaataga ttttaaaaac tggttgagtg catattactt | |
| | ttagcctcag aagacatcat gtatatttaa gaggcattta | |
| | actattgtaa attattctga tgactttaaa aaatgttcat | |
| | gctgagttgt atatttttaa ataaatttta ttagtttagt | |
| | ttaaaaaaag aaaagaaaat tattaatttt attaaaaaat | |
| | ctcctatatt taaaaaaaaa agagaaaaaG GCAGAGCTGG | |
| | GCTGGCTACA GTTACCACAA GAACATGGTC AGAGGAGGAA | |
| | GGGACTCTTA TACATACCTA TGACAGGAGA ACGGGAGACC | |
| | CAACATACTC GGGGGCCTAC CTTCAGAGAA CACAAGGCCA | |
| | GGGCAATACT CACAGSNNCT CATTGTTCGA CCCTGCCCTA | |
| | GTTCGACCTG TCAACATCAC TGAGCCCACC TTGGAGCTAC | |
| | TCCATTCATC CTGCGACCCC AATGCATTCC ACTCCACCAT | |
| | CCAGCTGTAC TGCTTCATTT ATGGCCACAT CCTAAATGAT | |
| | GTCTCTGTCA GCTGGCTAAT GGACGATCGG GAGATAACTG | |
| | ATACACTTGC ACAAACTGTT CTAATCAAGG AGGAAGGCAA | |
| | ACTAGCCTCT ACCTGCAGTA AACTCAACAT CACTGAGCAG | |
| | CAATGGATGT CTGAAAGCAC CTTCACCTGC AAGGTCACCT | |
| | CCCAAGGCGT AGACTATTTG GCCCACACTC GGAGATGCCC | |
| | AGGTAGGTCT ACACTCGCCT GATGCCCAGA CCTCAGAGTC | |
| | CTGAGGGAAA GGCAGGCTCT CACACAGCCC TTCCTCCCSN | |
| | NCGACAGATC ATGAGCCACG GGGTGTGATT ACCTACCTGA | |
| | TCCCACCCAG CCCCCTGGAC CTGTATCAAA ACGGTGCTCC CAA | |
| 8 | TATGAGATGA AAGTAAGCTG AGTTGGGCAG TTCTAGACTA | 9B |
| | TTCTAGGCTG TTCTGGGCTG ACCTGAACTG GGTGGGGTTG | |
| | AGCTGAATGA AGTAGGCTGT GCTGAGCTCT GCCAGGCTGG | |
| | ATGAGTTTAT TGATCTGAGT TGGACTGGCC TGGGCTGCAC | |
| | TAAATGGGAC TGAGATGAGA TTGGCCAGGC TAGGAGGGGT | |
| | TGAGCAAGGC TAAGCTAAGT TGTTCTGGAC TAGGCCAAAC | |
| | TATTAGAGGT CTTTTGGTTT AGTTGAACTT TCTGGTACTG | |
| | AACTAAACTG TCTTTAAGAT AGAATGCTCA AAATTATTTG | |
| | TGGGTGTTTT AACTGTCCTC AAAGAAGATT GTCCTGTTGT | |
| | AGGATACAAC AACAGCTACT AGCCAGACTG GGTAGGTAGA | |
| | GCCAATCGGG CCTAGCAGGA ATATCCTGTG CTTTCTGAGG | |
| | ACCTGGCACA GAGCTGAGCT GAGCCCCTCT CTCAGGAGAA | |
| | TGTCCTGGGC ATGTGGACAC TCTAGAGCAT CAAGGTGGCT | |
| | TCTGAAGTGG TTGTATTCTC TATGTGCTTT CTGGGATCCA | |
| | CGGAGGTCAT CTTGGAGGCA GAACACTGTG CAGGTTAGCC | |
| | TATGGTAAAG CAGAGAGCCT CATGTATCTG AAACCCAAAG | |
| | ATCCGATGAA TTGCCATTGT AAGTTTGCCT CTTCATCCAA | |
| | ACTCGTGCCC AGCTCTCCTG GAAGCCCCTG TGCTAAGCCA | |
| | GCTAGGGGCA GTGAGTGAGC AAAGCCTGAT GGGGTGTAAG | |
| | GAATCAGGGG GATCCCTAGG TCTGTGTTTG GGTTTAGTGA | |
| | ATAAAGACAA GACCCAGAAG GAATCTATGA CCAACAGCCC | |
| | TAGGAAACAA GAATCTCACC ATTCTGTCCT CAATGTGTCC | |
| | CAAAACAGAT TTAATGTGTC TCACCAAGAA ACTGGTGGTC | |
| | CTGGGAAAGC TCTGAATCCC CAGGCCCCAA GAGTGGGGAC | |
| | AGAAGAGACA ATGGCAATTC ATCGGATCTC TGGGCCACCA | |
| | AGCCCTGTGG GGTACCTATG TTCTGGACAT AAAGGACAAC | |
| | CTAGTCCCTC TGTCAACATT ACATAGCCTA CCTTAAAGCT | |
| | ACTCCATTCA TCCTGAGACC ATAATGGCTT CCAGTCTGCC | |
| | ACCCAGCTCT CATGCTTCAT TTCTGGACAT TCCCTAGATG | |
| | GTGTCACTGT CACCTGGTCT AAAGGACAGA CAGGAGATAC | |
| | CTCACACATA TCCACAAAAT TTCCCAATCA AGAAAGAGGG | |
| | CAAGTTTGCC TCTTTATCCA AACTTGTGCC CAGCTCTCCT | |
| | GGAAGCCCCT GTGCTAAGCC AGCGAGGGGC AGTGAGTGAG | |
| | CAAAGCCTGG CGGGGTGTAA GGAATCAGGG AGCTCCCTAG | |
| | GTCTGTGTTT GGGTTTAGAG AATAAAGACA AGACCCAGAA | |
| | TGAATCTAAC CATCTGTCTC CTAGACTGGA ATGGGGTCCC | |
| | CAGAGCCCTG CTCCTGTCAC AGCTGCCCTT AATCAGTTCC | |
| | CCATGCTGCA G---GCATGC AGTGATATAA ATAAGTCTAA | |
| | CCTAGGTCCT TCCTTTCTCA CAGCCTCTAT CAGGAACCCT | |
| | CAGCTCTACC CCTTGAAGCC CTGTAAAGGC ACTGCTTCCA | |
| | TGACCCTGGG CTGCCTGGTA AAGGACTACT TCCCTGGTCC | |
| | TGTGACTGTG ACCTGGTATT CAGACTCCCT GAACATGAGC | |
| | ACTGTGAACT TCCCTGCCCT TGGTTCTGAA CTCAAGGTCA | |
| | CCACCAGCCA AGTGACCAGC TGGGGCAAGT CAGCCAAGAA | |
| | CTTCACATGC CACGTGACAC ATCCTCCATC ATTCAACGAA | |
| | AGTAGGACTA TCCTAGGTAA GTAGGGATGG GCTGACAGTT | |
| | ACACTGTGTA TTCTCCCTTG GAGATGGAAC AGTTTCTGTC | |
| | TAATCAGGAA CTTGTCACAA TTTCCTTTCA TAGAGGACTT | |
| | CATAAGAGAT TTTTTTTTCT ACTTCTATCA TGTTTAGTGC | |
| | TCCAAATAGA TTTTTAAAAC TGGTTGAGTG CATATTACTT | |
| | TTAGCCTCAG AAGACATCAT GTATATTTAA GAGGCATTTA | |
| | ACTATTGTAA ATTATTCTGA TGACTTTAAA AAAAGTTAAT | |
| | GCTGAGTTGT ATATTTTTAA ATAAATTTTA TTAGTTTAGT | |
| | TTAAAAAAAG AAAAGAAAAT TATTAATTTT ATTTAAAAAT | |
| | CTCCTATATT TAAAAAAAAA AGAGAAAAAA GCAGAGCTGG | |
| | GCTGGCTACA GTTACCACAA GAACATGGTC AGAGGAGGAA | |
| | GGGACTCTTA TACATACCTA TGACAGGAGA ACGGGAGACC | |
| | CAACATACTC GGGGGCCTAC CTTCAGAGAA CACAAGGCCA | |
| | GGGCAATACT CACAG---CT CATTGTTCGA CCCTGCCCTA | |
| | GTTCGACCTG TCAACATCAC TGAGCCCACC TTGGAGCTAC | |
| | TCCATTCATC CTGCGACCCC AATGCTTTCC ACTCCACCAT | |
| | CCAGCTGTAC TGCTTCATTT ATGGCCACAT CCTAAATGAT | |
| | GTCTCTGTCA GCTGGCTAAT GGACGATCGG GAGATAACTG | |
| | ATACACTTGC ACAAACTGTT CTAATCAAGG AGGAAGGCAA | |
| | ACTAGCCTCT ACCTGCAGTA AACTCAACAT CACTGAGCAG | |
| | CAATGGATGT CTGAAAGCAC CTTCACCTGC AAGGTCACCT | |
| | CCCAAGGCGT AGACTATTTG GCCCACACTC GGAGATGCCC | |
| | AGGTAGGTCT ACACTCGCCT GATGTCCAGA CCTCAGAGTC | |
| | CTGAGGGAAA GGCAGGCTCT CACACAGCCC TTCCTCCC-- | |
| | -CGACAGATC ATGAGCCACG GGGTGTGATT ACCTACCTGA | |
| | TCCCACCCAG CCCCCTGGAC CTGTATCAAA ACGGTGCTCC CAA | |
| 9 | CTCTCCCTGT GGACCACAAA AGTTTATATT CTTCCTACAT | 10A |
| | ACAGTGCCCC TCCCCCCATG CCAACATCCC AAAGTCTCAT | |
| | CCTTCCAGCA TTCAGCTCTG CCCCAAGTCT CCTCCTAAGG | |
| | TCTGCTAGTC AAGTAAGGGT CAGATTGTGT GAGGTTTGTT | |
| | TTGAGATAGG ATTCAGTCCA CCTAGGCCAT AGCTGTCAGG | |
| | AGGAAGGGGA AGGAGAGAGG CACAGAAGGG AGAGGTATAC | |
| | CGTGATGAAC TGGGCAGACT GATAACATGC TGTAGAGCCA | |
| | AAAGCTGAGG GCAAGTGGGG TCCCCTCCCT CTCATGCTAA | |
| | GGTGACAGTT TCTAAGGGAG AGCAAGGGAT TTGGAGAAAG | |
| | AAGTGAAGGT TTGGTTCAGC ACTGGCCTTC CTGGTCCAGC | |
| | ACACTGCCCC TGCCTCAAAC TTTGCACATA CAGATCCCCC | |
| | ACTGTACTCC CCTCCTGCAT TTTCCCCACT ACCTTCAGCA | |
| | CACCACCAAC CTCTCTTCCA TGACTGCCTC CATCCCACCT | |
| | AGCATGGAGC CCCACTCCTG TGTGAGCCTA GTTCACTCAA | |
| | TGACCATGGG TGTCCATCTT CCAATGAAAC ATGAGCTCCA | |
| | TGGACAGGAA TATCCCTCCA GACCCATGTT CCTGCAGTTC | |
| | TATCTAACTG TTGGGCATTT ATGATGAAGT CACACCAGGT | |
| | CCCTCATTCC TAACTAACCT TCTAATCTGG CACAGTTATC | |
| | TGCTGGGAAC TAAGAGTGTG GTCAAAGTAA GATATGATGC | |
| | TGGCCGACCA GTAGGTCCAT GTGCTGGCTT TCAACTCTAA | |
| | ATCTTCCCCA CGCCTACCAT GTAGCCACAG GATCTTTCCC | |
| | CAAGGCAGGG TACAGATAGC ACATAGAGGA AGGAGGCCAA | |
| | GTGGATGGGG CTCTCCCGTA GCAGGTGTGG GAGAGGCAGG | |
| | TTGCACACAT GTGTGAATCC ATATGGTTCT GAGGTTTGGG | |
| | GGGTTTTTTT GTCCCAAAAT ATCATGTTCC AGAAGTACTT | |
| | GCTCCTTTGA CCTATACACC AGAGAAGAGA CCAAAAACTG | |
| | TGGTAGACAC AGGAGCAAGA ACAAAACCAT GCTGTTGTTT | |
| | TTCTACAGGC AAAACTTGTT GTCACCCTTA TTCCTGGCAA | |
| | AGCTATGTCG TCAAATTGCT AGTCCTGGTT GTAATTACAA | |
| | ATTTAATAAT ATATATAAAT ATATGTGCTA GACAACAGTT | |
| | AAAGAAATAA GGACAATGTT GGAGGGAGGA AATGAAAGTG | |
| | GGAAATGATA CAATAATTAT ATTTTCATCC CAAAGATAAA | |
| | GATATATGTT TTATTTCTGG TCCTGGGCTA AGGATATAGT | |
| | TCAATTGTTA AAGTACTTAT ATAGCATGCA GGAAGCCCTG | |
| | GATCTGATCT CCATAAATAA ACCAACTGTG GTCATTCATA | |
| | CCTGTAATTC CAGTACTCCA AAGGCAGACA CAGGAAGATG | |
| | GGGGCTTTGA AGTCATTCTT GGCTACATAG CAAATTAGAG | |
| | ACCAGCCTTA TCTTTAATAA TAATAATAAT CCTGACATTC | |
| | CACTTGAAGT AAATGTATCC AGTAATTGTA CCTGATACCA | |
| | TGCCATGTTA GATTTTATTT CAGGGTTTCA GAGAAAAGTA | |
| | CCCAGGGTTT TCCAGAGACG CACAAGTAGT GGAAGACAGC | |
| | AGACTGAGGA CTGGTAGATG AACGCATGAA CAACTTAGGA | |
| | CAGAGCTGGA GGACCATAAG GCTGATAGGT AGGTAGAAAC | |
| | AAGGATAACA CATAATAGGC TTACAGGTTT TATGGTTCAC | |
| | CTTGACTGGA CTAAGGGATG GAAGGCCTGT GGAGGTGTCT | |
| | GCAAAGGATT TTCCAGAAGT GTTAGGTCTT GGGGACTCTG | |
| | ATTTAACCGC ATGGTGCTGA ACAGTAGGAA GAAGGCCAGG | |
| | TGAAAGAAGC AGGTTACTGT GGGTAAGCCC GGGTAATTCT | |
| | ATCAGATCCT GGCACTGTAT TCCTGTTATC TCTACTTCCC | |
| | ATCAGTTGAG AAACAAACAG CCTCTTCCAC ACAACCCCGC | |
| | TGCCAAGATG GCCCTAAGCA CATAGGGCCA AACAAAAGTA | |
| | AATCTTTTCC CCCTTATGTT TCTTTGAGGT ATCAGGTCAT | |
| | CCTAGCTCCC GGCTCCAAGT CACACAGAGG GCTCCTATGA | |
| | GCAAGCATTT CCAACTCTAC CCTTTTTTTC TGTCACAAAC | |
| | CGCTGGCTAT TTGAGACATT TCAGACAGCT AAAAGATGGC | |
| | TCCCTTAGCA CTGATGGCTG AGTTCAAGAG GCTCACCTGC | |
| | TGGCTCGCAA ACCCAGAGGT AACCTGGGTT TGTGTTGCTT | |
| | TAGGCATAGA GAATGCTGAC ATCCAGCGGC TACGTGACTG | |
| | CTTCTGGGTT TTGGAACTTG TTCTTCAGTA CCATCCCCTG | |
| | CATCCAACTC TTCTCAGCTT GTAATCTTCT CAATATTTCC | |
| | ACCGTTCTTC ATCACCCTAG ATTCTTGCAG ATGCTGCCCT | |
| | CGATGGCTCA CTGGCTCCTC TACTCCCTGC CACTATCACC | |
| | CTCGGGGCCA GAAACCTGGA GCCATGTCTG CTCTTTGCAC | |
| | AGTAACACAG CCTACTCTAC CCATGACAAA GACCATAAGA | |
| | AGACTTGGAG ACCATTTAGG AAAAGCCTTT ATCATGGCCT | |
| | AATGCTGCAC ACGTGGATCA GGAGAAGCCT CAAAATATAG | |
| | TAGGGGGCAC ACTGTAGAGA CAGAATAGAG TCCATGATAC | |
| | GCTCATACAT GGATTATACT TCCAACAAGC ACTGCCCTGT | |
| | TTGTGCTCAT CTCCTGGTTC GACCAAGCAC AGTCTTCCCA | |
| | TATGAACCCA TCACAAGCCC TGCAGAATCA CAGATCACAG | |
| | GTCTTAGATA GGACCAGCTT TCTTTCTGAC AATAACCAGG | |
| | ATTTATTTGT TATTTCTTTT TATTGTATTT ATTTTTATTC | |
| | ATAATTTTAC ATCCCTCTCA CTGCCCCCTC CCAGTCATTC | |
| | CCTTCTGTTT TCCTCTCCCC ACTCCCCCTC CCTTTCTCCT | |
| | CTAAGCCAGT TAAGCCCCCT CTGTGTACCC CCCACCCTAA | |
| | TAATCAGGAG TTTTGAGCCA CCAGAGATGT TCTTCCTCCT | |
| | CTCTGACCTT GCTGAGAGCC TCTATGCCAA GGTCCTCTCG | |
| | AGCTGCATGT GAAGTCACTT GGAAGTCGTA GGTGAAGTGG | |
| | AGTTTTCCAG CTACAGTGCA GGCTGGAGCC CTGGTAACTA | |
| | GAACAAGGCT GTAGTTTCAG CAGCAGCCAT GATTGCAGGA | |
| | TACCTTGCAG CTCAAATATG GCCTCCTTGG GGCTCTGTGA | |
| | GGTATTCAAA GCATCTAGAA TCCCATGATG ACAGTTCTAC | |
| | CAGTCCCTAA AAGAAACCTA AGACGACTAG ATATAAGGAA | |
| | AGACCCACCT GAGTGCATCA AAAGGTCAAA TCAGCCTGGC | |
| | GCTCAACAGC TCATTTTACA TGAAGAAAAG GTGAACACTA | |
| | CCCTATTCCC AATAAAGACA TGTTGTTACA CTTACACTAA | |
| | CATCCTTGGC AGCCCTTAGC AGATGATCCT AGGGAGAGCT | |
| | GAGCAGTCTC ATCTACCTCA CCTCCACCCA GGCATCAAGT | |
| | TAACACTGTT CTAAGGTGCA CTTCTGAAAC TTACAGAGTT | |
| | GGGGTAGCAG TCAGACCTTT CCCTGACCCC CAAGATATGA | |
| | TCACACCCAC AACCACATAC ATGAGTTCGC AGACACTAAC | |
| | CGACACAGTG GATCTTAGAC CTGGCCCATT CCGGAATAGA | |
| | TCACTGTCAC AGTCACTTGA GTGAAGGAGC CACCCAAGGG | |
| | AATGGCTAAA GGACTG | |
| 10 | CGGGAGATAA CTGATACACT TGCACAAACT GTTCTAATCA | 10B |
| | AGGAGGAAGG CAAACTAGCC TCTACCTGCA GTAAACTCAA | |
| | CATCACTGAG CAGCAATGGA TGTCTGAAAG CACCTTCACC | |
| | TGCAAGGTCA CCTCCCAAGG CGTAGACTAT TTGGCCCACA | |
| | CTCGGAGATG CCCAGGTAGG TCTACACTCG CCTGATGCCC | |
| | AGACCTCAGA GTCCTGAGGG AAAGGCAGGC TCTCACACAG | |
| | CCCTTCCTCC CSNNCGACAG ATCATGAGCC ACGGGGTGTG | |
| | ATTACCTACC TGATCCCACC CAGCCCCCTG GACCTGTATC | |
| | AAAACGGTGC TCCCAAGCTT ACCTGTCTGG TGGTGGACCT | |
| | GGAAAGCGAG AAGAATGTCA ATGTGACGTG GAACCAAGAG | |
| | AAGAAGACTT CAGTCTCAGC ATCCCAGTGG TACACTAAGC | |
| | ACCACAATAA CGCCACAACT AGTATCACCT CCATCCTGCC | |
| | TGTAGTTGCC AAGGACTGGA TTGAAGGCTA CGGCTATCAG | |
| | TGCATAGTGG ACCACCCTGA TTTTCCCAAG CCCATTGTGC | |
| | GTTCCATCAC CAAGACCCCA GGTGAGTACA GGAGGTGGAG | |
| | AGTGGGCCAG CCCTSNNSMT CTTCATGTTC AGAGAACATG | |
| | GTTAACTGGT TAAGTCATGT CTGCCCACAG GCCAGCGCTC | |
| | AGCCCCCGAG GTATATGTGT TCCCACCACC AGAGGAGGAG | |
| | AGCGAGGACA AACGCACACT CACCTGTTTG ATCCAGAACT | |
| | TCTTCCCTGA GGATATCTCT GTGCAGTGGC TGGGGGATGG | |
| | CAAACTGATC TCAAACAGCC AGCACAGTAC CACAACACCC | |
| | CTGAAATCCA ATGGCTCCAA TCAAGGCTTC TTCATCTTCA | |
| | GTCGCCTAGA GGTCGCCAAG ACACTCTGGA CACAGAGAAA | |
| | ACAGTTCACC TGCCAAGTGA TCCATGAGGC ACTTCAGAAA | |
| | CCCAGGAAAC TGGAGAAAAC AATATCCACA AGCCTTGGTA | |
| | ACACCTCCCT CCGTCCCTCC TAGGCCTCCA TGTAGCTGTG | |
| | GTGGGGAAGG TGGATGACAG ACATCCGCTC ACTGTTGTAA | |
| | CACCAGGAAG CTACCCCAAT AAACACTCAG TGCCTGATTA | |
| | GAGCCCTGGG TGCCTGTTCT TGGGGAAGGC AGGTTATGGG | |
| | CAGAAATATC TTGGCCTGAA AGAAGGGACA CCCCAAGAGA | |
| | AGGACAGGAG TGAAGCATGG CTCACCCATC TGTCTATGTG | |
| | TTGAATATTT AACAAATAGG ACATCACAGG ACTTCAGCAT | |
| | AGTCCTTCAG CATACCCCTG GTCCTTCCTG CTCTTCACTG | |
| | GATATCATGC ACCTGATCTC TAGAGATGCA GCTAAAATGA | |
| | GCCAGTCTGA GAAGCCTCAG CACCCACCTC TCGGTCTTGC | |
| | AAGCTCCTGC TCCCAGGCTT TCCTGGATAC TAAACCCCTT | |
| | CAGGTAGAGA AACAGCCAAA GTCAACATCT AGGACGCAGG | |
| | ACTCAACATG GTCCTGCTCC TTCCCTCTCT ACTCAACAGC | |
| | CATTGAGGCT GAGCCCACCG CCCCAACCGC CTGCCTTGCC | |
| | AAATGATCAC GCCAGGCCTG GTGCTCCTCG ACTTACTACC | |
| | TAGACTCACT CCAACCCAAA TTCATCCCAA GGACCAGAAT | |
| | GGGCTGCCAG CCTCATACAG TCAGGTTCCC CCATCTATGA | |
| | CATGTTTTCA CACACATGCA CACACACACA CACACACACA | |
| | CACACACACA GAGCTAGGCT TCATTGAGCT CTCTGGTTTA | |
| | GCAATAGCCC AAAGCAAGCC ATACATCCAT CCCAGTTCCA | |
| | GAAGGATAAG AAAACCAGAA CCAAGACACA CCCACACCTA | |
| | TTCCATACCC AACCACCAGC ACATATGGCT TACACACCTG | |
| | AGATCAGTGG CTCCCATCAT GTACACACAC ATGCACACAA | |
| | AGGAGACCAT ACATACCCAT CATTTCCAGA GGTAAGTATC | |
| | TAACCTTTGG ATCTGAGATA CCTCTGAGGA ACACCAATGG | |
| | CAGAGTCGAC CAGCACCTCA GCCTCCAGAC TAAATCCTTA | |
| | CATTTTGGCC CACCCCAAGC CATGAGAGAT GGAGGAGGGT | |
| | AGAGGCCTGA GCTGCGGGAA AGCAGAGACA GGAAGATGGG | |
| | CTGTTTGGTG AGAGTAGTAA ACCAGACAAT GGGGAGACTA | |
| | AGGCAGGAGT AGAGCCCCTA CAAGGCCCAG AGTCTGCTTT | |
| | AGAGTCCATG TGTCCTGACC TGCCCCTCAG ATGCCACAAC | |
| | CAAGATTTCT GGTTCCAGAG CATGCATGCA GGCCCTAGAA | |
| | ATGGACCTAT GAGCTCAGAG CCTTCCTAGA GAGCCCTGGG | |
| | TACTCTCTGA ACAAAAGGCA ATTCTGTGTA GAGGCATCCT | |
| | GTGGCCAAAG ACCCTAAGAC AGTCATACAC ACACACAACA | |
| | CACACAACAC AGGTAGGCTT TATCATGCTC TTTGATTTAG | |
| | CAATAGCCCT GTTGATGGTG GGGGATACTG GGTCACTGTG | |
| | GGCACCGGAG TAGAAAGAGG GAATGAACAG TCAGTGGGGA | |
| | AAGGACATCT GCCTCTAGGG CTGAACAGAG ACTGGAGCAG | |
| | TCTCAGAGCA GGTGGGATGG GGACCTCTGC CACTCTAGCT | |
| | TCATCAGAAC TGCATGAGAC AAATATGGGG CCTACCCCCT | |
| | CCCCACTGTC ACCTGGAGTS NNMCTGGGGA AGCTAACTGG | |
| | CTGGTCCCAC CCCATCCCAG AGCTAGACCT CCAGGACCTA | |
| | TGTATTGAAG AGGTGGAGGG CGAGGAGCTG GAAGAGCTGT | |
| | GGACCAGTAT TTGTGTCTTC ATCACCCTGT TCCTGCTCAG | |
| | TGTGAGCTAT GGGGCCACTG TCACCGTCCT CAAGGTGGGA | |
| | TCCTGCACCT CAGCGGGTGG GTCTGGGAGG GCTAGGCCAA | |
| | GCCGCAGAGC CATCCTCACA TACSNNMACC TTTCCCCCAG | |
| | GTGAAGTGGG TCTTCTCCAC ACCGATGCAG GATACACCCC | |
| | AGACCTTCCA AGACTATGCC AACATCCTCC AGACCAGGGC | |
| | ATAGGTGCGA TGCCAGCACC CATGCAGGCC TGCAGCCATG | |
| | TGTGCTTGAG CCTCCTGAGG TGCCTGTTTG CCCGGGTGAT | |
| | AGGAGGGAGC AGAGACCCCT AAAGGCACCA ACGTTGATGA | |
| | GATATCAGCA TCCCAGAAAG TTGCAGCTCA GAGCACCTAG | |
| | GTGGGCTGTC CTACACAGAT ACTTTGAGAC AAAGCTTAGA | |
| | AGAACATCTA TCCCTCATCG ATTTGACCTA CCAGATGCTA | |
| | GCCACCTGGG CTAATCCCAG GTCTATGGGC ATCAGGACCA | |
| | CTCCATTTTG ACTGAATAAC CACAAAAACA CAAGAACTCA | |
| | GAGTCTAGAG TTCCCACTAG ACCCCACCTA GAGCACAGAG | |
| | TCAAAGCTGG GACACTCAGA ATCAACCCTA AGTCCAGACG | |
| | CTGGCTCCTC AGAAGG | |
| 11 | CTCTGGCCCTGCTTATTGTTG | 11A |
| 12 | CCTGATAGAGGCTGTGAGAAAGGAAGGACC | 11B |
| 13 | AGACCTGGGAATGTATGGTT | 11C |
| 14 | TAGGTTAGACTTATTTATATCACTGCATGC | 11D |
| 15 | GTTGAGAGCCCTAGTAAGCG | 11E |
| 16 | TTGAGAGCCCTAGTAAGCG | 11F |
| 17 | TGAGCTCAGCTATGCTACGCGTGTTG | 11G |
| 18 | GCCCGATTGGCTCTACCTACCCAGTCTGGC | 11H |
| 19 | gggaagagat agaaggaagg ctaggtgggg caagacgagg | 12 |
| | gaactaaagc cactgtgctg ctggggacac tgtggacact | |
| | gatggacaga aagggagtga tcagtctgtg gacaggaggg | |
| | ggaggggcaa ggatgatgct gacagagagt cacagtggag | |
| | tccgtagcag gaaagagaga gagcgcccag tgtagtccta | |
| | aggcttagga agttgcaact gcctcctctc cttccagagg | |
| | atcactcact gccatctagc atagaactca gatgacccag | |
| | aaccagcagc tcagcccaac ctgtgtgtca cagaagaatc | |
| | aggcccagtc aggctagaca caaaggctct tggccctcat | |
| | gctgtgaggg aggtacacac tgggggcaca ccacaaacag | |
| | ttggagcaga ggcttctcac ccctattttt ccctctgaac | |
| | aatagttgct tccagggaac tctgcattta cccctcaggc | |
| | tcccacccat gtctgttagg ctgaaggcca agcctgtcac | |
| | ctcagacaga cagtggatct gaaagacaga aggccgtgca | |
| | agaccacaat tcccttgaat ctcacactct gtcttcccaa | |
| | agttcctaac tgcatctgac ctttctgggc cagcctctca | |
| | gcctgcctgg ctctgccact atcaggaaga tctctaatat | |
| | cttccaaatg caattaaaca cgctcctgtg aaagtcagac | |
| | ttggcaaagc ctaagtccct tcggtccctt tcagtgggac | |
| | caacgactct gagcagccag ggtccaaggg atggggctct | |
| | cattttcttc cccaaatctc tgtgtgcctc tctcaagact | |
| | caagactcac aagcaaaatt agtggctcct atagcttgta | |
| | tgtatgtttt cttggaactc ctaggaacca tgggcctaca | |
| | gagacatcag agtgtagagg gaatccctga acccagaaga | |
| | tgaccttgct ctacaaagct gcagctgaga cagacactac | |
| | tagtacccca tgaaagctgc tgagccaaag cccagccctc | |
| | acaccatctt taccctcatc cctcccctca gtgcagacat | |
| | agaccacagg cctggaagag acgttagctg tttctacaca | |
| | gctccgtgaa acccagtcac aacccagatg cgctctgtcc | |
| | ttctggactc cttgccagag tagcaggtag aggacctcaa | |
| | gctgaaagat aatcacttgt gagtgggcac cagggaaggc | |
| | cactgtccct cgcatgccag ctccaaagct gatacaggaa | |
| | ctagggtgcc tctatcagag gccctgcaat gtcatatctg | |
| | gcccacaggc tgttcctctt tgtgcaccat taataactta | |
| | caaagtgaca gccacactcc cctgaagggc tgccaaagga | |
| | acagaaaaag caatggctag ggtctagtcc tgcttcaggg | |
| | cagtgacact ccaaaggggc aggcatggtg actgcacaca | |
| | cgcacatgca aggctttaat acgagagcta tgcaaggaga | |
| | cctgggatca gacgatggag aatagagagc cttgaccaga | |
| | gtgtgcaggt gtgtctccta gaaagaggcc tcacctgaga | |
| | ccccactgtg ccttagtcaa cttcccaaga acagaatcaa | |
| | aagggaactt ccaaggctgc taaggccggg ggttcccacc | |
| | ccacttttag ctgagggcac tgaggcagag cggcccctag | |
| | gtactaccat ctgggcatga attaatggtt actagagatt | |
| | cacaacgcct gggagcctgc acagggggca gaagatggct | |
| | tcgaataaga acagtctggc cagccactca cttatcagag | |
| | gacctcaggt attacaaccc atgggaccct gagcaaaagg | |
| | gtttgcctaa ggagaaggga caaacaggtt acagggtcct | |
| | gggtggggaa ggggacacct gggctgcctt ctaatgtgga | |
| | cagtctcttg gtcaccgaat gtccttcagc tatcacttcc | |
| | ctgcactaag gcacacaggt attagaaact gctatagcta | |
| | tccatgaaga caggggactg tggatctcaa ccagagaggg | |
| | ctgaaccaag ataaactgaa tatgttgtga gaaactcaaa | |
| | aactgcagga gaggctggag aggaatcggc cagcaagcca | |
| | tcagacaaaa atgcaatgac aaatgtcaga tccagcaaat | |
| | gacagcaagg aattgccctg tgatgaacta acaaccaaga | |
| | ggactgtcca cagctgggct gacccaggca gcactgggct | |
| | aaattgggtg ggatctgtgc tgccctgggc tggtatgagc | |
| | caggatgagc caagtgaagt gggctggact agtttgggct | |
| | ggactggcct ggagtaggct aaaccagttt aaactagagt | |
| | aggctgggct gaggtgaatc agactaggct agactagtct | |
| | gagctgggtt aagcagagct gtgctgggct ggtatgagct | |
| | ggtccaagtt gggctaaaca gagctgggcc aggctagtat | |
| | gagctggtct gaactacact aagcaggact aggctgggct | |
| | gagctgagct ggactggctg gacttggctg agatgtgttg | |
| | agctgggtta agtatggctg ggctgggctg gcctgggctg | |
| | ggctggactg gattggtatg agctggtcca agttgggcta | |
| | agcagagctg ggccaggctg gtatgagctg gtctaaactg | |
| | aactaagtag ggctgggcta agctgagctg gtctacacta | |
| | gcctgacctg agctagggta ggctggactg ggctgagcta | |
| | agttgcactg ggcaggggtg ggctggaccg agctgatttg | |
| | agctgggatg ggctgagatg ggttcagcag gcctaagcag | |
| | gcctagctgg gtttagctag atttagctag gcaaggctga | |
| | gctaggctgg gcggggcggg gctaggctgg gcagggctgg | |
| | actgagctag cttttgtata ttcggttgaa atgggttggt | |
| | ctggtctgga ctgaactgac tgagctgggc tagcctgagc | |
| | tcgatggggg gtatactcag ctgagatggg ctggtctggc | |
| | tagactgaac tggattgggc taggctgagc taggctgacc | |
| | tgaactggcc tggtctgggc tggactgggc agggctggtc | |
| | tcagctagac tacactgagt taacctgggc tggaccatac | |
| | tgggttaaac taggttgcac tggctgggtt agacttggct | |
| | gagctgggct tggctgagct gagtcaagat ggtctgagtt | |
| | gatttgagtt ggctaagcta agctgagcta cactgaacta | |
| | ggcaaggctg ggctggaaag gtctgggtta agttaggagg | |
| | gacttggctt ggcttagctg ggccaagcta ggctgaactg | |
| | ggctgaactg agctgagctg ggctgagctg ggctgagctg | |
| | ggctgagctg ggcaaggcta aactggaatg gactgaattg | |
| | gcctaagatg ggcccggcta agctaagtaa ggctgccctg | |
| | aactgagcag gactggcctg gcctggattg acctggcatg | |
| | agcttaactt gactagacta gtctatcttg ggtgaactgg | |
| | gctaagcagg actaatctgg cctgatctga gctagactga | |
| | actaggctaa gctgagctga gtttagcttg gctgaactgg | |
| | gctgggctgc actgaactgt attgagctat gtagaactga | |
| | gctggtcttg tctgaggtgg gttgggctgg tctgggctga | |
| | accagattgc actagactga gcttagctgg acctggctga | |
| | gctggactgc attgtgctaa actggctctc tttagaccga | |
| | gcttagctgg actggactga gctaggttgg gtgggctgat | |
| | ctaagctgag ctaggctggt ctcacctgag gaatgctgtg | |
| | ctgtgctgag ctgaactaaa ctgagctcag ctaaggaagt | |
| | gtgagctaga ctgagctgag ctaggctggg ttgggctgaa | |
| | ctgagctacc ttgggtggac taggctgagc tgagctgggt | |
| | tgagctgagc tatagatttg gttggactgg actggattgg | |
| | gctaaactga actggtttgg ggtaggctgg gatgagctgg | |
| | actgagctag gctgtactgg tctgagctaa actaagttga | |
| | gtggggctaa gaggagctga gtgaggctgg gctggaatga | |
| | gctaggctag ggttgtgagc tagggttgta ctggtctaag | |
| | ctgagtttag ctgagagagg ctgggctaga cttccataag | |
| | gtggctgagt catactacag tgcactgagc tgtgttgagc | |
| | ttaacttgga ttaagtggaa tgggttgagc tggctgaact | |
| | gggctgaact gagataaact agactgagct gggacacgct | |
| | gggacgagct ggaacgagct agaattactg ttctaatctg | |
| | atctgggctg aggtaaactg ggcctggttg agctctacta | |
| | ggctaagtag agttgagcta tgagatgaaa gtaagctgag | |
| | ttgggcagtt ctagactatt ctaggctgtt ctgggctgac | |
| | ctgaactggg tggggttgag ctgaatgaag taggctgtgc | |
| | tgagctctgc caggctggat gagtttattg atctgagttg | |
| | gactggcctg ggctgcacta aatgggactg agatgagatt | |
| | ggccaggcta ggaggggttg agcaaggcta agctaagttg | |
| | ttctggacta ggccaaacta ttagaggtct tttggtttag | |
| | ttgaactttc tggtactgaa ctaaactgtc tttaagatag | |
| | aatgctcaaa attatttgtg ggtgttttaa ctgtcctcaa | |
| | agaagattgt cctgttgtag gatacaacaa cagctactag | |
| | ccagactggg taggtagagc caatcgggcc tagcaggaat | |
| | atcctgtgct ttctgaggac ctggcacaga gctgagctga | |
| | gcccctctct caggagaatg tcctgggcat gtggacactc | |
| | tagagcatca aggtggcttc tgaagtggtt gtattctcta | |
| | tgtgctttct gggatccacg gaggtcatct tggaggcaga | |
| | acactgtgca ggttagccta tggtaaagca gagagcctca | |
| | tgtatctgaa acccaaagat ccgatgaatt gccattgtaa | |
| | gtttgcctct tcatccaaac tcgtgcccag ctctcctgga | |
| | agcccctgtg ctaagccagc taggggcagt gagtgagcaa | |
| | agcctgatgg ggtgtaagga atcaggggga tccctaggtc | |
| | tgtgtttggg tttagtgaat aaagacaaga cccagaagga | |
| | atctatgacc aacagcccta ggaaacaaga atctcaccat | |
| | tctgtcctca atgtgtccca aaacagattt aatgtgtctc | |
| | accaagaaac tggtggtcct gggaaagctc tgaatcccca | |
| | ggccccaaga gtggggacag aagagacaat ggcaattcat | |
| | cggatctctg ggccaccaag ccctgtgggg tacctatgtc | |
| | ctggacataa aggacaacct agtccctctg tcaacattac | |
| | atagcctacc ttaaagctac tccattcatc ctgagaccat | |
| | aatggcttcc agtctgccac ccagctctca tgcttcattt | |
| | ctggacattc cctagatggt gtcactgtca cctggtctaa | |
| | aggacagaca ggagatacct cacacatatc cacaaaattt | |
| | cccaatcaag aaagagggca agtttgcctc tttatccaaa | |
| | cttgtgccca gctctcctgg aagcccctgt gctaagccag | |
| | cgaggggcag tgagtgagca aagcctggcg gggtgtaagg | |
| | aatcagggag ctccctaggt ctgtgtttgg gtttagagaa | |
| | taaagacaag acccagaatg aatctaacca tctgtctcct | |
| | agactggaat ggggtcccca gagccctgct cctgtcacag | |
| | ctgcccttaa tcagttcccc atgctgcagg catgcagtga | |
| | tataaataag tctaacctag gtccttcctt tctcacagcc | |
| | tctatcagga accctcagct ctaccccttg aagccctgta | |
| | aaggcactgc ttccatgacc ctgggctgcc tggtaaagga | |
| | ctacttccct ggtcctgtga ctgtgacctg gtattcagac | |
| | tccctgaaca tgagcactgt gaacttccct gcccttggtt | |
| | ctgaactcaa ggtcaccacc agccaagtga ccagctgggg | |
| | caagtcagcc aagaacttca catgccacgt gacacatcct | |
| | ccatcattca acgaaagtag gactatccta ggtaagtagg | |
| | gatgggctga cagttacact gtgtattctc ccttggagat | |
| | ggaacagttt ctgtctaatc aggaacttgt cacaatttcc | |
| | tttcatagag gacttcataa gagatttttt tttctacttc | |
| | tatcatgttt agtgctccaa atagattttt aaaactggtt | |
| | gagtgcatat tacttttagc ctcagaagac atcatgtata | |
| | tttaagaggc atttaactat tgtaaattat tctgatgact | |
| | ttaaaaaaag ttaatgctga gttgtatatt tttaaataaa | |
| | ttttattagt ttagtttaaa aaaagaaaag aaaattatta | |
| | attttattta aaaatctcct atatttaaaa aaaaaagaga | |
| | aaaaagcaga gctgggctgg ctacagttac cacaagaaca | |
| | tggtcagagg aggaagggac tcttatacat acctatgaca | |
| | ggagaacggg agacccaaca tactcggggg cctaccttca | |
| | gagaacacaa ggccagggca atactcacag ctcattgttc | |
| | gaccctgccc tagttcgacc tgtcaacatc actgagccca | |
| | ccttggagct actccattca tcctgcgacc ccaatgcttt | |
| | ccactccacc atccagctgt actgcttcat ttatggccac | |
| | atcctaaatg atgtctctgt cagctggcag atct | |
| 20 | taatggacga tcgggagata actgatacac ttgcacaaac | N/A |
| | tgttctaatc aaggaggaag gcaaactagc ctctacctgc | |
| | agtaaactca acatcactga gcagcaatgg atgtctgaaa | |
| | gcaccttcac ctgcaaggtc acctcccaag gcgtagacta | |
| | tttggcccac actcggagat gcccaggtag gtctacactc | |
| | gcctgatgtc cagacctcag agtcctgagg gaaaggcagg | |
| | ctctcacaca gcccttcctc cccgacagat catgagccac | |
| | ggggtgtgat tacctacctg atcccaccca gccccctgga | |
| | cctgtatcaa aacggtgctc ccaagctt | |

N/A = not applicable

### CITATION LIST

### Patent Literature

Karasuyama et al., U.S. Pat. No. 6,118,044 —Sep. 12, 2000—Transgenic non-human animal allergy models

### Non-Patent Literature

Gerstein et al., Isotype switching of an immunoglobulin heavy chain transgene occurs by DNA recombination between different chromosomes, Cell (1990) 63:537-548
Liu et al., A highly efficient recombineering-based method for generating conditional knockout mutations. Genome Research (2003) vol. 13 (3) pp. 476-84.
Pan et al., Characterization of Human y4 Switch Region Polymorphisms Suggests a Meiotic Recombinational Hot Spot Within the Ig Locus: Influence of S Region Length on IgG4 Production, J. Immunol. (1998) 161:3520-3526.
Schmidtz, J and Radbruch, A, Immunoglobulin Class Switching in Encyclopedia of Immunology, Delves and Roitt (eds.), pages 1302-1306.
Szurek et al., Complete nucleotide sequence of the murine gamma-3 switch region and analysis of switch recombination in two gamma-3 expressing hybridomas, J. Immunol. 135:620-626 (1985).
Warming et al., Mol. Cell. Biol. (2006) 26 (18):6913-22) for subsequent use in embryonic stem (ES) cell targeting, resulting in “pBlight-DTA-IgE”.
Waterston et al., Initial sequencing and comparative analysis of the mouse genome, Nature. (2002) 420(6915):520-62.
Zarrin et al., Influence of switch region length on immunoglobulin class switch recombination, Proc Natl Acad Sci (2005) 102(7):2466-2470.
Zarrin et al., Antibody Class Switching Mediated by Yeast Endonuclease-Generated DNA Breaks, Science (2007) 315:377-381
Zarrin et al., Sgamma3 switch sequences function in place of endogenous Sgamma1 to mediate antibody class switching, (2008) J. Exp. Med. 205, 1567

What is claimed is:

1. A targeting vector comprising:
a. a fragment of DNA homologous to the 5′ end of the switch region to be altered (the 5′ arm/acceptor) is selected from the group consisting of at least 1500 nucleotides, at least 1800 nucleotides, at least 2000 nucleotides, at least 2200 nucleotides and at least 2400 nucleotides corresponding to Nucleotides 25470628 to 25468161 of NCBI Accession number NT_{—}166318 (Mus musculus chromosome 12 genomic contig, strain C57BU6J);
b. a selectable gene marker;
c. a desired/donor DNA sequence encoding a donor switch region; and
d. a second fragment of DNA homologous to the 3′ end of the switch region to be altered (the 3′ arm/acceptor) is selected from the group consisting of at least 1500 nucleotides, at least 1800 nucleotides, at least 2000 nucleotides, at least 2200 nucleotides, at least 2400 nucleotides and at least 2800 nucleotides corresponding to Nucleotides 25470628 to 25468161 of NCBI Accession Number NT_{—}166318 (Mus musculus chromosome 12 genomic contig, strain C57BU6J).

2. The targeting vector of claim 1 wherein the 5′ arm comprises SEQ ID NO:4 or 5.

3. The targeting vector of claim 1 wherein the 5′ arm is homologous to a region 3′ of the endogenous Iε and 5′ of the endogenous Sε.

4. The targeting vector of claim 1 wherein the 3′ arm comprises SEQ ID NO:7 or 8.

5. The targeting vector of claim 1 wherein the selectable gene marker is selected from the group consisting of Neomycin and thymidine kinase.

6. The targeting vector of claim 1 wherein the selectable gene marker is Neomycin.

7. The targeting vector of claim 1 wherein the selectable gene marker is flanked by loxp sites.

8. The targeting vector of claim 1 wherein the desired switch region is from a mouse.

9. The targeting vector of claim 1 wherein the desired switch region is selected from Sμ, Sγ1, Sγ2a, Sγ2b and Sγ3.

10. The targeting vector of claim 1 wherein the desired switch region is the HindIII/NheI fragment containing most of mouse Sm region.

11. The targeting vector of claim 1 wherein the desired switch region comprises Nucleotides corresponding to 25617172 to 25615761 of NCBI Accession Number NT_{—}166318 (Mus musculus chromosome 12 genomic contig, strain C57BL/6J).

12. A method for producing an altered embryonic stem cell in vitro, comprising the steps of:
a. Altering the genomic DNA in said cell to enhance the probability of CSR to express the Cc selected from
i. increasing the Sε length by adding at least one additional Sε copy in tandem with the endogenous Sε region;
ii. Sε region substitution; and
b. Selecting the cell for correctly altered genomic DNA.

13. The method according to claim 12 wherein the alteration is a substitution of a switch region selected from Sμ, Sγ1, Sγ2a, Sγ2b and Sγ3 for the Sε region.

14. The method according to claim 12 wherein the alteration is a substitution of a Sμ region for the Sε region.

15. A method for producing an altered embryonic stem cell in vitro, comprising the steps of:
a. Using the vector according to claim 1 to exchange the Sμ for the Sε region
b. Selecting the cell for correctly altered genomic DNA.

16. The method according to claim 15 wherein the alteration is a substitution of a switch region selected from Sμ, Sγ1, Sγ2a, Sγ2b and Sγ3 for the Sε region.

17. The method according to claim 15 wherein the alteration is a substitution of a Sμ region for the Sε region.

18. The method of claim 15, wherein the ESC are from a mouse strain selected from BALB/c or C57BL/6.

19. A non-human animal wherein
a. At least one allele of the IgH locus has been altered to enhance the rate of IgE expression/production/secretion/relative to a non-altered allele; and
b. Has an IgE profile selected from the group consisting of
i. The IgE fraction of all serum antibodies is greater than 0.04%;
ii. The IgE serum concentration is above 4,000 ng/ml
iii. The IgG/IgE ratio is less than 10.

20. A non-human mammal having a genome which has been altered to express an IgE molecule at a level greater than 4000 ng/ml.

21. A non-human mammal having an IgG/IgE ratio that is between 0.1 and 10.

22. A non-human mammal having an unchallenged (i.e., resting) IgE serum concentration of between 100 ng/mL and 10000 ng/mL.

23. A non-human mammal having a challenged (i.e., activated) IgE serum concentration of between 1000 ng/mL and 1000000 ng/mL.

24. The animal model of claim 19, wherein the animal model is a nonhuman vertebrate.

25. The animal model of claim 19, wherein the animal model is a mouse, rat, guinea pig, rabbit, or primate.

26. The non-human animal/mammal model of claim 19, wherein the genome of said non-animal has had the Sε region of the IgH locus altered to express/produce more IgE.

27. The non-human animal/mammal model of claim 19, wherein the alteration is by gene targeting.

28. A method of testing an allergy therapy using the animal model of claim 19 comprising exposing said animal to an allergen prior to, simultaneous with or after the administration of said method of treatment for allergic disorders and evaluating the IgE response.

29. The method of claim 28 wherein the IgE response is less than without the allergy therapy.

30. The method of claim 28, wherein the test animal and the control animal are littermates.

31. Use of a compound identified by the method of claim 28 as a medicament for the treatment of an allergy.

32. A cell line obtainable from the animal model of claim 19.

33. A cell isolated from an animal model of claim 19.

34. A process for making a non-human animal model, said process comprising:
a. microinjecting linearized fragments of plasmids encoding SEQ ID NO:6 (Sμ) into a fertilized egg of a mouse such that the fragment is incorporated in the genomic DNA upstream from and operably linked to the Cε-encoding region,
b. transferring said fertilized egg to the oviduct of a female mouse which has previously been treated to induce pseudopregnancy, and
c. allowing said egg to develop in the uterus of the female mouse.

35. A recombinant mouse comprising in its germline a modified genome wherein said modification comprises at least one allele of the IgH locus altered to enhance the rate of IgE production.

36. The recombinant mouse of claim 35 wherein the alteration comprises a replacing the Sε with the Sμ region or a functional portion thereof.

37. The recombinant mouse of claim 35 wherein the Sμ functional portion is between at least 1 kb and 10 kb in length.


SEQUENCE LISTING (20 sequences)

SEQ ID NO: 1 - DNA, 11 bp, Mus musculus
gggctgggctg

SEQ ID NO: 2 - DNA, 10 bp, Mus musculus
gagctgagct

SEQ ID NO: 3 - DNA, 2055 bp, Mus musculus
tgggttaagcagagctgtgctgggctggtatgagctggtccaagttgggctaaacagagctgggccaggctagtatgagctggtctgaactacactaagcaggactaggctgggctgagctgagctggactggctggacttggctgagatgtgttgagctgggttaagtatggctgggctgggctggcctgggctgggctggactggattggtatgagctggtccaagttgggctaagcagagctgggccaggctggtatgagctggtctaaactgaactaagtagggctgggctaagctgagctggtctacactagcctgacctgagctagggtaggctggactgggctgagctaagttgcactgggcaggggtgggctggaccgagctgatttgagctgggatgggctgagatgggttcagcaggcctaagcaggcctagctgggtttagctagatttagctaggcaaggctgagctaggctgggcggggcggggctaggctgggcagggctggactgagctagcttttgtatattcggttgaaatgggttggtctggtctggactgaactgactgagctgggctagcctgagctcgatggggggtatactcagctgagatgggctggtctggctagactgaactggattgggctaggctgagctaggctgacctgaactggcctggtctgggctggactgggcagggctggtctcagctagactacactgagttaacctgggctggaccatactgggttaaactaggttgcactggctgggttagacttggctgagctgggcttggctgagctgagtcaagatggtctgagttgatttgagttggctaagctaagctgagctacactgaactaggcaaggctgggctggaaaggtctgggttaagttaggagggacttggcttggcttagctgggccaagctaggctgaactgggctgaactgagctgagctgggctgagctgggctgagctgggctgagctgggcaaggctaaactggaatggactgaattggcctaagatgggcccggctaagctaagtaaggctgccctgaactgagcaggactggcctggcctggattgacctggcatgagcttaacttgactagactagtctatcttgggtgaactgggctaagcaggactaatctggcctgatctgagctagactgaactaggctaagctgagctgagtttagcttggctgaactgggctgggctgcactgaactgtattgagctatgtagaactgagctggtcttgtctgaggtgggttgggctggtctgggctgaaccagattgcactagactgagcttagctggacctggctgagctggactgcattgtgctaaactggctctctttagaccgagcttagctggactggactgagctaggttgggtgggctgatctaagctgagctaggctggtctcacctgaggaatgctgtgctgtgctgagctgaactaaactgagctcagctaaggaagtgtgagctagactgagctgagctaggctgggttgggctgaactgagctaccttgggtggactaggctgagctgagctgggttgagctgagctatagatttggttggactggactggattgggctaaactgaactggtttggggtaggctgggatgagctggactgagctaggctgtactggtctgagctaaactaagttgagtggggctaagaggagctgagtgaggctgggctggaatgagctaggctagggttgtgagctagggttgtactggtctaagctgagtttagctgagagaggctgggctagacttccataaggtggctgagtcatactacagtgcactgagctgtgttgagcttaacttggattaagtggaatgggttgagctggctgaactgggctgaactgagataaactagactgagctgggacacgctgggacgagctggaacgagctagaattactgttctaatctgatctgggctgaggtaaactgggcctggttgagctctactaggctaagtagagttgagct

SEQ ID NO: 4 - DNA, 2471 bp, Mus musculus
  modified_base (1506)..(1507): a, c, t, g, unknown or other
gatccctgtgaagccctgggccatgggaagagatagaaggaaggctaggtggggcaagacgagggaactaaagccactgtgctgctggggacactgtggacactgatggacagaaagggagtgatcagtctgtggacaggagggggaggggcaaggatgatgctgacagagagtcacagtggagtccgtagcaggaaagagagagagcgcccagtgtagtcctaaggcttaggaagttgcaactgcctcctctccttccagaggatcactcactgccacctagcatagaactcagaggacccagaaccagcagctcagcccaacctgtgtgtcacagaagaatcaggcccggtcaggctagacacaaaggctcttggccctcatgctgtgagggaggtacacactggaggcacaccacaaacagttggagcagaggcttctcgcccctatttttccctctgaacaatagttgcttccagggaactctgcatttacccctcaggctcccacccatgtctattaggctgaaggccaagcctgtcacctcagacagacagtgtatctgaaagacagaaggccgtgcaagaccacaattcccttgaatctcacactctgtcttcccaaagttcctaactgcatctgacctttctgggccagcctctcagcctgcctggctctgccactatcaggaagatctctaatatcttccaaatgcaattaaacacgctcctgtgaaagtcagacttggcatagcctaagtcccttcggtccctttcactgggaccaacgaccctgagcagccagggtccaagggatggggctctcattttcttccccaaatctctgtgtgcctctctcaagactcaagactcacaagcaaaattagtggctcctatagtttgtatgtatgttttcttagaactcctaggaaccatgggcctacagagacatcagagtgtagagggaatccctgaacccagaagatgaccttgctctacaaagctgcagctgagacagacactactagtaccccatgaaagctgctgagccaaagcccagccctcacaccatctttaccctcatccctcccctcagtgcagacatagaccacaggcctggaagagacgttagctgtttctacacagctccgtgaaacccagtcacaacccagatgtgctctgtccttctggactccttgccagagtagcaggtagaggacctcaagctgaaagataatcacttgtgagtgggcaccagggaaggccactgtccctcgcatgccagctccaaagctgatacaggaactagggtgcctctatcagaggccctgcaatgtcatatctggcccacaggctgttcctctttgtgcaccattaataacttacaaagtgacagccacactcccctgaagggctgccaaaggaacagaaaaagcaatggcgagggtctagtcctgcctcagggcagtgacactccaaaggggcaggcatggtgactgcacgcasnncacacatgcaaggctttaatacgagagctatgcaaggagacctgggatcagacgatggagaatagagagccttgaccagagtgtgcaggtgtgtctcctagaaagaggcctcacctgagaccccactgtgccttagtcaacttcccaagaacagaatcaaaagggaacttccaaggctgctaaggccgggggttcccaccccacttttagctgagggcactgaggcagagcggcccctaggtactaccatctgggcatgaattaatggttactagagattcacaacgcctgggagcctgcacagggggcagaagatggcttcgaataagaacagtctggccagccactcacttatcagaggacctcaggtattacaacccatgggaccctgagcaaaagggtttgcctaaggagaagggacaaacaggttacagggtcctgggtggggaaggggacacctgggctgccttctaatgtggacagtctcttgaccaccgaatgtccttcagctatcacttccctgcactaaggcacacaggtattagaaactgctatagctattcatgaagacgggggactgtggatctcaaccagagagggctgaaccaagataaactgaatatgttgtgagaaactcaaaaactgcaggagaggctggagaggaatcggccagcaagccatcagacaagaatgcaatgacaaatgtcagatccagcaaatgacagcaaggaattgccctgtgatgaactaacaaccaagaggactgtccacagctgggctgacccaggcagcactgggctaaattgggtgggatctgtgctgccctgggctggtatgagccaggatgagccaagtgaagtgggctggactagtttgggctggactggcctggagtaggctaaaccagtttaaactagagtaggctgggctgaggtgaatcagactaggctagactagtctgagc

SEQ ID NO: 5 - DNA, 2468 bp, Mus musculus
gatccctgtgaagccctgggccatgggaagagatagaaggaaggctaggtggggcaagacgagggaactaaagccactgtgctgctggggacactgtggacactgatggacagaaagggagtgatcagtctgtggacaggagggggaggggcaaggatgatgctgacagagagtcacagtggagtccgtagcaggaaagagagagagcgcccagtgtagtcctaaggcttaggaagttgcaactgcctcctctccttccagaggatcactcactgccatctagcatagaactcagatgacccagaaccagcagctcagcccaacctgtgtgtcacagaagaatcaggcccagtcaggctagacacaaaggctcttggccctcatgctgtgagggaggtacacactgggggcacaccacaaacagttggagcagaggcttctcacccctatttttccctctgaacaatagttgcttccagggaactctgcatttacccctcaggctcccacccatgtctgttaggctgaaggccaagcctgtcacctcagacagacagtggatctgaaagacagaaggccgtgcaagaccacaattcccttgaatctcacactctgtcttcccaaagttcctaactgcatctgacctttctgggccagcctctcagcctgcctggctctgccactatcaggaagatctctaatatcttccaaatgcaattaaacacgctcctgtgaaagtcagacttggcaaagcctaagtcccttcggtccctttcagtgggaccaacgactctgagcagccagggtccaagggatggggctctcattttcttccccaaatctctgtgtgcctctctcaagactcaagactcacaagcaaaattagtggctcctatagcttgtatgtatgttttcttggaactcctaggaaccatgggcctacagagacatcagagtgtagagggaatccctgaacccagaagatgaccttgctctacaaagctgcagctgagacagacactactagtaccccatgaaagctgctgagccaaagcccagccctcacaccatctttaccctcatccctcccctcagtgcagacatagaccacaggcctggaagagacgttagctgtttctacacagctccgtgaaacccagtcacaacccagatgcgctctgtccttctggactccttgccagagtagcaggtagaggacctcaagctgaaagataatcacttgtgagtgggcaccagggaaggccactgtccctcgcatgccagctccaaagctgatacaggaactagggtgcctctatcagaggccctgcaatgtcatatctggcccacaggctgttcctctttgtgcaccattaataacttacaaagtgacagccacactcccctgaagggctgccaaaggaacagaaaaagcaatggctagggtctagtcctgcttcagggcagtgacactccaaaggggcaggcatggtgactgcacacacgcacatgcaaggctttaatacgagagctatgcaaggagacctgggatcagacgatggagaatagagagccttgaccagagtgtgcaggtgtgtctcctagaaagaggcctcacctgagaccccactgtgccttagtcaacttcccaagaacagaatcaaaagggaacttccaaggctgctaaggccgggggttcccaccccacttttagctgagggcactgaggcagagcggcccctaggtactaccatctgggcatgaattaatggttactagagattcacaacgcctgggagcctgcacagggggcagaagatggcttcgaataagaacagtctggccagccactcacttatcagaggacctcaggtattacaacccatgggaccctgagcaaaagggtttgcctaaggagaagggacaaacaggttacagggtcctgggtggggaaggggacacctgggctgccttctaatgtggacagtctcttggtcaccgaatgtccttcagctatcacttccctgcactaaggcacacaggtattagaaactgctatagctatccatgaagacaggggactgtggatctcaaccagagagggctgaaccaagataaactgaatatgttgtgagaaactcaaaaactgcaggagaggctggagaggaatcggccagcaagccatcagacaaaaatgcaatgacaaatgtcagatccagcaaatgacagcaaggaattgccctgtgatgaactaacaaccaagaggactgtccacagctgggctgacccaggcagcactgggctaaattgggtgggatctgtgctgccctgggctggtatgagccaggatgagccaagtgaagtgggctggactagtttgggctggactggcctggagtaggctaaaccagtttaaactagagtaggctgggctgaggtgaatcagactaggctagactagtctgagc

SEQ ID NO: 6 - DNA, 1440 bp, Mus musculus
agctcaccccagctcagctcagctcaccccagctcagcccagctcagcccagctcagcccagctcagcccagctcagcccagctcagctcagctcagcccagctcagcccagctcaccccagctcagctcagctcaccccagctcagccctgctcagcccagctcagctcaccccagctcagctcagctcagctcaccccagctcagctcagctcaccccagctcagctcagctcagcccagctcagctcagctcaccccagctcagctcaccccagctcagcccagctcagcccagctcagcccagctcaccccagctcagcccagctcagctcagctcaccccagctcagctcagctcagctcagctcagctcagctcagctcagctcagctcagctcagctcagctcagctcagctcagctcagctcagcccagctcaccccagctcagctcaccccagctcagctcagctcaccccagctcagctcagctcagctcagctcaccccagctcagcccagctcaccccagctcagcccagctcagctcactccagctcagctcagctcaccccagctcaccccagctcagctcagctcagctcaccccagctcagctcagctcagctcagctcagctcaccccagctcagctcagctcaccccagctcagctcaccccagctcagctcagctcaccccagctcagctcaccccagctcaccccagctcagctcagctcaccccagctcagctcagctcaccccagctcagctcaccccagctcagcccagctcagcccagctcagcccagctcaccccagctcagcccagctcagctcactccagctcagctcagctcaccccagctcatcccagcttaccccagctcagctcagctcaccccagctcagcccagctcaccctagctcagccaagctcagctcagctcaccccagcttagctcagctcacccagctcagctcaccctagctcagctcagtctagctcagcccagctcagctcaccccatctcaccccatctcagctactccagagtatctcatttcagatcagctcaccccaacacagcgtagcatagctgagctcaccccagctcatctcagctcagaacagtccagtgtaggcagtagagtttagctctattcaacctagattaatgaagttcattccagtttggctcatctcggttaagccagcctagtttagcttagcggcccagctcattccagttcattacagtctacttcattttggctcaagcccagctttgcttacctcagagtaatcacctcagtttagcgcattttagaagcactcagagaagcccacccatctcagctcagctgtgctttttagagcctcgcttactagggctctcaaccttgttcccttaattttgctcagcaagctttatgagt

SEQ ID NO: 7 - DNA, 2842 bp, Mus musculus
  modified_base (1493)..(1494): a, c, t, g, unknown or other
tatgagatgaaagtaagctgagttgggcagttctagactattctaggctgttctgggctgacctgaactgggtggggttgagctgaatgaagtaggctgtgctgagctctgccaggctggatgagtttattgatctgagttggactggcctgggctgcactaaatgggactgagatgagattggccaggctaggagggattgagcaaggctaagctaagttgttctggactaggccaaactattagaggtcttttggtttagttgaactttctggtactgaactaaactgtctttaagatagaatgctcaaaattatttgtgggtgttttaactgtcctcaaagaagattgtcctgttgtaggatacaacaacagctactagccagactgggtaggtagagccaatcgggcctagcaggaatatcctgtgctttctgaggacctggcacagagctgagctgagcccctctctcaggagaatgtcctgggcatgtggacactctagagcatcaaggtggcttctgaagtggttgtattctctatgtgctttctgggatccacggaggtcatcttggaggcagaacactgtgcaggttagcctatggtaaagcagagagcctcatgtatctgaaacccaaagatccgattaattgccattgtaagtttgcctcttcatccaaactcgtgcccagctctcctggaagcccctgtgctaagccagctaggggcagtgagtgagcaaagcctgatggggtgtaaggaatcagggggatccctaggtctgtgtttgggtttagtgaataaagacaagacccagaaggaatctatgaccaacagccctaggaaacaagaatctcaccattctgtcctcaatgtgtcccaaaacagatttaatgtgtctcaccaagaaactggtggtcctgggaaagctctgaatccccaggccccaagagtggggacagaagagacaatggcaattcatcggatctctgggccaccaagccctgtggggtacctatgtcctggacataaaggacaacctagtccctctgtcaacattacatagcctaccttaaagctactccattcatcctgagaccataatggcttccagtctgccacccagctctcatgcttcatttctggacattccctagatggcgtcactgtcacctggtctaaaggacagacaggagatacctcacacatatccacaaaatttcccaatcaagaaagagggcaagtttgcctctttatccaaacttgtgcccagctctcctggaagcccctgtgctaagccagcgaggggcagtgagtgagcaaagcctggcggggtgtaaggaatcagggagctccctaggtctgtgtttgggtttagagaataaagacaagacccagaaggaatctaaccatctgtctcctagactggaatggggtccccagagccctgctcctgtcacagctgcccttaatcagttccccatgctgcagsnngcatgcagtgatataaataagtctaacctaggtccttcctttctcacagcctctatcaggaaccctcagctctaccccttgaagccctgtaaaggcactgcttccatgaccctgggctgcctggtaaaggactacttccctggtcctgtgactgtgacctggtattcagactccctgaacatgagcactgtgaacttccctgccctcggttctgaactcaaggtcaccaccagccaagtgaccagctggggcaagtcagccaagaacttcacatgccacgtgacacatcctccatcattcaacgaaagtaggactatcctaggtaagtagggatgggctgacagttacactgtgtattctcccttggagatggaacagtttctgtctaatcaggaacttgtcacaatttcctttcatagaggacttcataagagattttttttctacttctatcatgtttagtgatccaaatagattttaaaaactggttgagtgcatattacttttagcctcagaagacatcatgtatatttaagaggcatttaactattgtaaattattctgatgactttaaaaaatgttcatgctgagttgtatatttttaaataaattttattagtttagtttaaaaaaagaaaagaaaattattaattttattaaaaaatctcctatatttaaaaaaaaaagagaaaaaggcagagctgggctggctacagttaccacaagaacatggtcagaggaggaagggactcttatacatacctatgacaggagaacgggagacccaacatactcgggggcctaccttcagagaacacaaggccagggcaatactcacagsnnctcattgttcgaccctgccctagttcgacctgtcaacatcactgagcccaccttggagctactccattcatcctgcgaccccaatgcattccactccaccatccagctgtactgcttcatttatggccacatcctaaatgatgtctctgtcagctggctaatggacgatcgggagataactgatacacttgcacaaactgttctaatcaaggaggaaggcaaactagcctctacctgcagtaaactcaacatcactgagcagcaatggatgtctgaaagcaccttcacctgcaaggtcacctcccaaggcgtagactatttggcccacactcggagatgcccaggtaggtctacactcgcctgatgcccagacctcagagtcctgagggaaaggcaggctctcacacagcccttcctcccsnncgacagatcatgagccacggggtgtgattacctacctgatcccacccagccccctggacctgtatcaaaacggtgctcccaa

SEQ ID NO: 8 - DNA, 2834 bp, Mus musculus
tatgagatgaaagtaagctgagttgggcagttctagactattctaggctgttctgggctgacctgaactgggtggggttgagctgaatgaagtaggctgtgctgagctctgccaggctggatgagtttattgatctgagttggactggcctgggctgcactaaatgggactgagatgagattggccaggctaggaggggttgagcaaggctaagctaagttgttctggactaggccaaactattagaggtcttttggtttagttgaactttctggtactgaactaaactgtctttaagatagaatgctcaaaattatttgtgggtgttttaactgtcctcaaagaagattgtcctgttgtaggatacaacaacagctactagccagactgggtaggtagagccaatcgggcctagcaggaatatcctgtgctttctgaggacctggcacagagctgagctgagcccctctctcaggagaatgtcctgggcatgtggacactctagagcatcaaggtggcttctgaagtggttgtattctctatgtgctttctgggatccacggaggtcatcttggaggcagaacactgtgcaggttagcctatggtaaagcagagagcctcatgtatctgaaacccaaagatccgatgaattgccattgtaagtttgcctcttcatccaaactcgtgcccagctctcctggaagcccctgtgctaagccagctaggggcagtgagtgagcaaagcctgatggggtgtaaggaatcagggggatccctaggtctgtgtttgggtttagtgaataaagacaagacccagaaggaatctatgaccaacagccctaggaaacaagaatctcaccattctgtcctcaatgtgtcccaaaacagatttaatgtgtctcaccaagaaactggtggtcctgggaaagctctgaatccccaggccccaagagtggggacagaagagacaatggcaattcatcggatctctgggccaccaagccctgtggggtacctatgtcctggacataaaggacaacctagtccctctgtcaacattacatagcctaccttaaagctactccattcatcctgagaccataatggcttccagtctgccacccagctctcatgcttcatttctggacattccctagatggtgtcactgtcacctggtctaaaggacagacaggagatacctcacacatatccacaaaatttcccaatcaagaaagagggcaagtttgcctctttatccaaacttgtgcccagctctcctggaagcccctgtgctaagccagcgaggggcagtgagtgagcaaagcctggcggggtgtaaggaatcagggagctccctaggtctgtgtttgggtttagagaataaagacaagacccagaatgaatctaaccatctgtctcctagactggaatggggtccccagagccctgctcctgtcacagctgcccttaatcagttccccatgctgcaggcatgcagtgatataaataagtctaacctaggtccttcctttctcacagcctctatcaggaaccctcagctctaccccttgaagccctgtaaaggcactgcttccatgaccctgggctgcctggtaaaggactacttccctggtcctgtgactgtgacctggtattcagactccctgaacatgagcactgtgaacttccctgcccttggttctgaactcaaggtcaccaccagccaagtgaccagctggggcaagtcagccaagaacttcacatgccacgtgacacatcctccatcattcaacgaaagtaggactatcctaggtaagtagggatgggctgacagttacactgtgtattctcccttggagatggaacagtttctgtctaatcaggaacttgtcacaatttcctttcatagaggacttcataagagatttttttttctacttctatcatgtttagtgctccaaatagatttttaaaactggttgagtgcatattacttttagcctcagaagacatcatgtatatttaagaggcatttaactattgtaaattattctgatgactttaaaaaaagttaatgctgagttgtatatttttaaataaattttattagtttagtttaaaaaaagaaaagaaaattattaattttatttaaaaatctcctatatttaaaaaaaaaagagaaaaaagcagagctgggctggctacagttaccacaagaacatggtcagaggaggaagggactcttatacatacctatgacaggagaacgggagacccaacatactcgggggcctaccttcagagaacacaaggccagggcaatactcacagctcattgttcgaccctgccctagttcgacctgtcaacatcactgagcccaccttggagctactccattcatcctgcgaccccaatgctttccactccaccatccagctgtactgcttcatttatggccacatcctaaatgatgtctctgtcagctggctaatggacgatcgggagataactgatacacttgcacaaactgttctaatcaaggaggaaggcaaactagcctctacctgcagtaaactcaacatcactgagcagcaatggatgtctgaaagcaccttcacctgcaaggtcacctcccaaggcgtagactatttggcccacactcggagatgcccaggtaggtctacactcgcctgatgtccagacctcagagtcctgagggaaaggcaggctctcacacagcccttcctccccgacagatcatgagccacggggtgtgattacctacctgatcccacccagccccctggacctgtatcaaaacggtgctcccaa

SEQ ID NO: 9 - DNA, 3736 bp, Mus musculus
ctctccctgtggaccacaaaagtttatattcttcctacatacagtgcccctccccccatgccaacatcccaaagtctcatccttccagcattcagctctgccccaagtctcctcctaaggtctgctagtcaagtaagggtcagattgtgtgaggtttgttttgagataggattcagtccacctaggccatagctgtcaggaggaaggggaaggagagaggcacagaagggagaggtataccgtgatgaactgggcagactgataacatgctgtagagccaaaagctgagggcaagtggggtcccctccctctcatgctaaggtgacagtttctaagggagagcaagggatttggagaaagaagtgaaggtttggttcagcactggccttcctggtccagcacactgcccctgcctcaaactttgcacatacagatcccccactgtactcccctcctgcattttccccactaccttcagcacaccaccaacctctcttccatgactgcctccatcccacctagcatggagccccactcctgtgtgagcctagttcactcaatgaccatgggtgtccatcttccaatgaaacatgagctccatggacaggaatatccctccagacccatgttcctgcagttctatctaactgttgggcatttatgatgaagtcacaccaggtccctcattcctaactaaccttctaatctggcacagttatctgctgggaactaagagtgtggtcaaagtaagatatgatgctggccgaccagtaggtccatgtgctggctttcaactctaaatcttccccacgcctaccatgtagccacaggatctttccccaaggcagggtacagatagcacatagaggaaggaggccaagtggatggggctctcccgtagcaggtgtgggagaggcaggttgcacacatgtgtgaatccatatggttctgaggtttggggggtttttttgtcccaaaatatcatgttccagaagtacttgctcctttgacctatacaccagagaagagaccaaaaactgtggtagacacaggagcaagaacaaaaccatgctgttgtttttctacaggcaaaacttgttgtcacccttattcctggcaaagctatgtcgtcaaattgctagtcctggttgtaattacaaatttaataatatatataaatatatgtgctagacaacagttaaagaaataaggacaatgttggagggaggaaatgaaagtgggaaatgatacaataattatattttcatcccaaagataaagatatatgttttatttctggtcctgggctaaggatatagttcaattgttaaagtacttatatagcatgcaggaagccctggatctgatctccataaataaaccaactgtggtcattcatacctgtaattccagtactccaaaggcagacacaggaagatgggggctttgaagtcattcttggctacatagcaaattagagaccagccttatctttaataataataataatcctgacattccacttgaagtaaatgtatccagtaattgtacctgataccatgccatgttagattttatttcagggtttcagagaaaagtacccagggttttccagagacgcacaagtagtggaagacagcagactgaggactggtagatgaacgcatgaacaacttaggacagagctggaggaccataaggctgataggtaggtagaaacaaggataacacataataggcttacaggttttatggttcaccttgactggactaagggatggaaggcctgtggaggtgtctgcaaaggattttccagaagtgttaggtcttggggactctgatttaaccgcatggtgctgaacagtaggaagaaggccaggtgaaagaagcaggttactgtgggtaagcccgggtaattctatcagatcctggcactgtattcctgttatctctacttcccatcagttgagaaacaaacagcctcttccacacaaccccgctgccaagatggccctaagcacatagggccaaacaaaagtaaatcttttcccccttatgtttctttgaggtatcaggtcatcctagctcccggctccaagtcacacagagggctcctatgagcaagcatttccaactctaccctttttttctgtcacaaaccgctggctatttgagacatttcagacagctaaaagatggctcccttagcactgatggctgagttcaagaggctcacctgctggctcgcaaacccagaggtaacctgggtttgtgttgctttaggcatagagaatgctgacatccagcggctacgtgactgcttctgggttttggaacttgttcttcagtaccatcccctgcatccaactcttctcagcttgtaatcttctcaatatttccaccgttcttcatcaccctagattcttgcagatgctgccctcgatggctcactggctcctctactccctgccactatcaccctcggggccagaaacctggagccatgtctgctctttgcacagtaacacagcctactctacccatgacaaagaccataagaagacttggagaccatttaggaaaagcctttatcatggcctaatgctgcacacgtggatcaggagaagcctcaaaatatagtagggggcacactgtagagacagaatagagtccatgatacgctcatacatggattatacttccaacaagcactgccctgtttgtgctcatctcctggttcgaccaagcacagtcttcccatatgaacccatcacaagccctgcagaatcacagatcacaggtcttagataggaccagctttctttctgacaataaccaggatttatttgttatttctttttattgtatttatttttattcataattttacatccctctcactgccccctcccagtcattcccttctgttttcctctccccactccccctccctttctcctctaagccagttaagccccctctgtgtaccccccaccctaataatcaggagttttgagccaccagagatgttcttcctcctctctgaccttgctgagagcctctatgccaaggtcctctcgagctgcatgtgaagtcacttggaagtcgtaggtgaagtggagttttccagctacagtgcaggctggagccctggtaactagaacaaggctgtagtttcagcagcagccatgattgcaggataccttgcagctcaaatatggcctccttggggctctgtgaggtattcaaagcatctagaatcccatgatgacagttctaccagtccctaaaagaaacctaagacgactagatataaggaaagacccacctgagtgcatcaaaaggtcaaatcagcctggcgctcaacagctcattttacatgaagaaaaggtgaacactaccctattcccaataaagacatgttgttacacttacactaacatccttggcagcccttagcagatgatcctagggagagctgagcagtctcatctacctcacctccacccaggcatcaagttaacactgttctaaggtgcacttctgaaacttacagagttggggtagcagtcagacctttccctgacccccaagatatgatcacacccacaaccacatacatgagttcgcagacactaaccgacacagtggatcttagacctggcccattccggaatagatcactgtcacagtcacttgagtgaaggagccacccaagggaatggctaaaggactg

SEQ ID NO: 10 - DNA, 3416 bp, Mus musculus
  modified_base (253)..(254): a, c, t, g, unknown or other
cgggagataactgatacacttgcacaaactgttctaatcaaggaggaaggcaaactagcctctacctgcagtaaactcaacatcactgagcagcaatggatgtctgaaagcaccttcacctgcaaggtcacctcccaaggcgtagactatttggcccacactcggagatgcccaggtaggtctacactcgcctgatgcccagacctcagagtcctgagggaaaggcaggctctcacacagcccttcctcccsnncgacagatcatgagccacggggtgtgattacctacctgatcccacccagccccctggacctgtatcaaaacggtgctcccaagcttacctgtctggtggtggacctggaaagcgagaagaatgtcaatgtgacgtggaaccaagagaagaagacttcagtctcagcatcccagtggtacactaagcaccacaataacgccacaactagtatcacctccatcctgcctgtagttgccaaggactggattgaaggctacggctatcagtgcatagtggaccaccctgattttcccaagcccattgtgcgttccatcaccaagaccccaggtgagtacaggaggtggagagtgggccagccctsnnsmtcttcatgttcagagaacatggttaactggttaagtcatgtctgcccacaggccagcgctcagcccccgaggtatatgtgttcccaccaccagaggaggagagcgaggacaaacgcacactcacctgtttgatccagaacttcttccctgaggatatctctgtgcagtggctgggggatggcaaactgatctcaaacagccagcacagtaccacaacacccctgaaatccaatggctccaatcaaggcttcttcatcttcagtcgcctagaggtcgccaagacactctggacacagagaaaacagttcacctgccaagtgatccatgaggcacttcagaaacccaggaaactggagaaaacaatatccacaagccttggtaacacctccctccgtccctcctaggcctccatgtagctgtggtggggaaggtggatgacagacatccgctcactgttgtaacaccaggaagctaccccaataaacactcagtgcctgattagagccctgggtgcctgttcttggggaaggcaggttatgggcagaaatatcttggcctgaaagaagggacaccccaagagaaggacaggagtgaagcatggctcacccatctgtctatgtgttgaatatttaacaaataggacatcacaggacttcagcatagtccttcagcatacccctggtccttcctgctcttcactggatatcatgcacctgatctctagagatgcagctaaaatgagccagtctgagaagcctcagcacccacctctcggtcttgcaagctcctgctcccaggctttcctggatactaaaccccttcaggtagagaaacagccaaagtcaacatctaggacgcaggactcaacatggtcctgctccttccctctctactcaacagccattgaggctgagcccaccgccccaaccgcctgccttgccaaatgatcacgccaggcctggtgctcctcgacttactacctagactcactccaacccaaattcatcccaaggaccagaatgggctgccagcctcatacagtcaggttcccccatctatgacatgttttcacacacatgcacacacacacacacacacacacacacacacagagctaggcttcattgagctctctggtttagcaatagcccaaagcaagccatacatccatcccagttccagaaggataagaaaaccagaaccaagacacacccacacctattccatacccaaccaccagcacatatggcttacacacctgagatcagtggctcccatcatgtacacacacatgcacacaaaggagaccatacatacccatcatttccagaggtaagtatctaacctttggatctgagatacctctgaggaacaccaatggcagagtcgaccagcacctcagcctccagactaaatccttacattttggcccaccccaagccatgagagatggaggagggtagaggcctgagctgcgggaaagcagagacaggaagatgggctgtttggtgagagtagtaaaccagacaatggggagactaaggcaggagtagagcccctacaaggcccagagtctgctttagagtccatgtgtcctgacctgcccctcagatgccacaaccaagatttctggttccagagcatgcatgcaggccctagaaatggacctatgagctcagagccttcctagagagccctgggtactctctgaacaaaaggcaattctgtgtagaggcatcctgtggccaaagaccctaagacagtcatacacacacacaacacacacaacacaggtaggctttatcatgctctttgatttagcaatagccctgttgatggtgggggatactgggtcactgtgggcaccggagtagaaagagggaatgaacagtcagtggggaaaggacatctgcctctagggctgaacagagactggagcagtctcagagcaggtgggatggggacctctgccactctagcttcatcagaactgcatgagacaaatatggggcctaccccctccccactgtcacctggagtsnnmctggggaagctaactggctggtcccaccccatcccagagctagacctccaggacctatgtattgaagaggtggagggcgaggagctggaagagctgtggaccagtatttgtgtcttcatcaccctgttcctgctcagtgtgagctatggggccactgtcaccgtcctcaaggtgggatcctgcacctcagcgggtgggtctgggagggctaggccaagccgcagagccatcctcacatacsnnmacctttcccccaggtgaagtgggtcttctccacaccgatgcaggatacaccccagaccttccaagactatgccaacatcctccagaccagggcataggtgcgatgccagcacccatgcaggcctgcagccatgtgtgcttgagcctcctgaggtgcctgtttgcccgggtgataggagggagcagagacccctaaaggcaccaacgttgatgagatatcagcatcccagaaagttgcagctcagagcacctaggtgggctgtcctacacagatactttgagacaaagcttagaagaacatctatccctcatcgatttgacctaccagatgctagccacctgggctaatcccaggtctatgggcatcaggaccactccattttgactgaataaccacaaaaacacaagaactcagagtctagagttcccactagaccccacctagagcacagagtcaaagctgggacactcagaatcaaccctaagtccagacgctggctcctcagaagg

SEQ ID NO: 11 - DNA, 21 bp, Artificial Sequence
  Description of Artificial Sequence Synthetic oligonucleotide
ctctggccctgcttattgttg

SEQ ID NO: 12 - DNA, 30 bp, Artificial Sequence
  Description of Artificial Sequence Synthetic oligonucleotide
cctgatagaggctgtgagaaaggaaggacc

SEQ ID NO: 13 - DNA, 20 bp, Artificial Sequence
  Description of Artificial Sequence Synthetic oligonucleotide
agacctgggaatgtatggtt

SEQ ID NO: 14 - DNA, 30 bp, Artificial Sequence
  Description of Artificial Sequence Synthetic oligonucleotide
taggttagacttatttatatcactgcatgc

SEQ ID NO: 15 - DNA, 20 bp, Artificial Sequence
  Description of Artificial Sequence Synthetic oligonucleotide
gttgagagccctagtaagcg

SEQ ID NO: 16 - DNA, 19 bp, Artificial Sequence
  Description of Artificial Sequence Synthetic oligonucleotide
ttgagagccctagtaagcg

SEQ ID NO: 17 - DNA, 26 bp, Artificial Sequence
  Description of Artificial Sequence Synthetic oligonucleotide
tgagctcagctatgctacgcgtgttg

SEQ ID NO: 18 - DNA, 30 bp, Artificial Sequence
  Description of Artificial Sequence Synthetic oligonucleotide
gcccgattggctctacctacccagtctggc

SEQ ID NO: 19 - DNA, 6994 bp, Mus musculus
gggaagagatagaaggaaggctaggtggggcaagacgagggaactaaagccactgtgctgctggggacactgtggacactgatggacagaaagggagtgatcagtctgtggacaggagggggaggggcaaggatgatgctgacagagagtcacagtggagtccgtagcaggaaagagagagagcgcccagtgtagtcctaaggcttaggaagttgcaactgcctcctctccttccagaggatcactcactgccatctagcatagaactcagatgacccagaaccagcagctcagcccaacctgtgtgtcacagaagaatcaggcccagtcaggctagacacaaaggctcttggccctcatgctgtgagggaggtacacactgggggcacaccacaaacagttggagcagaggcttctcacccctatttttccctctgaacaatagttgcttccagggaactctgcatttacccctcaggctcccacccatgtctgttaggctgaaggccaagcctgtcacctcagacagacagtggatctgaaagacagaaggccgtgcaagaccacaattcccttgaatctcacactctgtcttcccaaagttcctaactgcatctgacctttctgggccagcctctcagcctgcctggctctgccactatcaggaagatctctaatatcttccaaatgcaattaaacacgctcctgtgaaagtcagacttggcaaagcctaagtcccttcggtccctttcagtgggaccaacgactctgagcagccagggtccaagggatggggctctcattttcttccccaaatctctgtgtgcctctctcaagactcaagactcacaagcaaaattagtggctcctatagcttgtatgtatgttttcttggaactcctaggaaccatgggcctacagagacatcagagtgtagagggaatccctgaacccagaagatgaccttgctctacaaagctgcagctgagacagacactactagtaccccatgaaagctgctgagccaaagcccagccctcacaccatctttaccctcatccctcccctcagtgcagacatagaccacaggcctggaagagacgttagctgtttctacacagctccgtgaaacccagtcacaacccagatgcgctctgtccttctggactccttgccagagtagcaggtagaggacctcaagctgaaagataatcacttgtgagtgggcaccagggaaggccactgtccctcgcatgccagctccaaagctgatacaggaactagggtgcctctatcagaggccctgcaatgtcatatctggcccacaggctgttcctctttgtgcaccattaataacttacaaagtgacagccacactcccctgaagggctgccaaaggaacagaaaaagcaatggctagggtctagtcctgcttcagggcagtgacactccaaaggggcaggcatggtgactgcacacacgcacatgcaaggctttaatacgagagctatgcaaggagacctgggatcagacgatggagaatagagagccttgaccagagtgtgcaggtgtgtctcctagaaagaggcctcacctgagaccccactgtgccttagtcaacttcccaagaacagaatcaaaagggaacttccaaggctgctaaggccgggggttcccaccccacttttagctgagggcactgaggcagagcggcccctaggtactaccatctgggcatgaattaatggttactagagattcacaacgcctgggagcctgcacagggggcagaagatggcttcgaataagaacagtctggccagccactcacttatcagaggacctcaggtattacaacccatgggaccctgagcaaaagggtttgcctaaggagaagggacaaacaggttacagggtcctgggtggggaaggggacacctgggctgccttctaatgtggacagtctcttggtcaccgaatgtccttcagctatcacttccctgcactaaggcacacaggtattagaaactgctatagctatccatgaagacaggggactgtggatctcaaccagagagggctgaaccaagataaactgaatatgttgtgagaaactcaaaaactgcaggagaggctggagaggaatcggccagcaagccatcagacaaaaatgcaatgacaaatgtcagatccagcaaatgacagcaaggaattgccctgtgatgaactaacaaccaagaggactgtccacagctgggctgacccaggcagcactgggctaaattgggtgggatctgtgctgccctgggctggtatgagccaggatgagccaagtgaagtgggctggactagtttgggctggactggcctggagtaggctaaaccagtttaaactagagtaggctgggctgaggtgaatcagactaggctagactagtctgagctgggttaagcagagctgtgctgggctggtatgagctggtccaagttgggctaaacagagctgggccaggctagtatgagctggtctgaactacactaagcaggactaggctgggctgagctgagctggactggctggacttggctgagatgtgttgagctgggttaagtatggctgggctgggctggcctgggctgggctggactggattggtatgagctggtccaagttgggctaagcagagctgggccaggctggtatgagctggtctaaactgaactaagtagggctgggctaagctgagctggtctacactagcctgacctgagctagggtaggctggactgggctgagctaagttgcactgggcaggggtgggctggaccgagctgatttgagctgggatgggctgagatgggttcagcaggcctaagcaggcctagctgggtttagctagatttagctaggcaaggctgagctaggctgggcggggcggggctaggctgggcagggctggactgagctagcttttgtatattcggttgaaatgggttggtctggtctggactgaactgactgagctgggctagcctgagctcgatggggggtatactcagctgagatgggctggtctggctagactgaactggattgggctaggctgagctaggctgacctgaactggcctggtctgggctggactgggcagggctggtctcagctagactacactgagttaacctgggctggaccatactgggttaaactaggttgcactggctgggttagacttggctgagctgggcttggctgagctgagtcaagatggtctgagttgatttgagttggctaagctaagctgagctacactgaactaggcaaggctgggctggaaaggtctgggttaagttaggagggacttggcttggcttagctgggccaagctaggctgaactgggctgaactgagctgagctgggctgagctgggctgagctgggctgagctgggcaaggctaaactggaatggactgaattggcctaagatgggcccggctaagctaagtaaggctgccctgaactgagcaggactggcctggcctggattgacctggcatgagcttaacttgactagactagtctatcttgggtgaactgggctaagcaggactaatctggcctgatctgagctagactgaactaggctaagctgagctgagtttagcttggctgaactgggctgggctgcactgaactgtattgagctatgtagaactgagctggtcttgtctgaggtgggttgggctggtctgggctgaaccagattgcactagactgagcttagctggacctggctgagctggactgcattgtgctaaactggctctctttagaccgagcttagctggactggactgagctaggttgggtgggctgatctaagctgagctaggctggtctcacctgaggaatgctgtgctgtgctgagctgaactaaactgagctcagctaaggaagtgtgagctagactgagctgagctaggctgggttgggctgaactgagctaccttgggtggactaggctgagctgagctgggttgagctgagctatagatttggttggactggactggattgggctaaactgaactggtttggggtaggctgggatgagctggactgagctaggctgtactggtctgagctaaactaagttgagtggggctaagaggagctgagtgaggctgggctggaatgagctaggctagggttgtgagctagggttgtactggtctaagctgagtttagctgagagaggctgggctagacttccataaggtggctgagtcatactacagtgcactgagctgtgttgagcttaacttggattaagtggaatgggttgagctggctgaactgggctgaactgagataaactagactgagctgggacacgctgggacgagctggaacgagctagaattactgttctaatctgatctgggctgaggtaaactgggcctggttgagctctactaggctaagtagagttgagctatgagatgaaagtaagctgagttgggcagttctagactattctaggctgttctgggctgacctgaactgggtggggttgagctgaatgaagtaggctgtgctgagctctgccaggctggatgagtttattgatctgagttggactggcctgggctgcactaaatgggactgagatgagattggccaggctaggaggggttgagcaaggctaagctaagttgttctggactaggccaaactattagaggtcttttggtttagttgaactttctggtactgaactaaactgtctttaagatagaatgctcaaaattatttgtgggtgttttaactgtcctcaaagaagattgtcctgttgtaggatacaacaacagctactagccagactgggtaggtagagccaatcgggcctagcaggaatatcctgtgctttctgaggacctggcacagagctgagctgagcccctctctcaggagaatgtcctgggcatgtggacactctagagcatcaaggtggcttctgaagtggttgtattctctatgtgctttctgggatccacggaggtcatcttggaggcagaacactgtgcaggttagcctatggtaaagcagagagcctcatgtatctgaaacccaaagatccgatgaattgccattgtaagtttgcctcttcatccaaactcgtgcccagctctcctggaagcccctgtgctaagccagctaggggcagtgagtgagcaaagcctgatggggtgtaaggaatcagggggatccctaggtctgtgtttgggtttagtgaataaagacaagacccagaaggaatctatgaccaacagccctaggaaacaagaatctcaccattctgtcctcaatgtgtcccaaaacagatttaatgtgtctcaccaagaaactggtggtcctgggaaagctctgaatccccaggccccaagagtggggacagaagagacaatggcaattcatcggatctctgggccaccaagccctgtggggtacctatgtcctggacataaaggacaacctagtccctctgtcaacattacatagcctaccttaaagctactccattcatcctgagaccataatggcttccagtctgccacccagctctcatgcttcatttctggacattccctagatggtgtcactgtcacctggtctaaaggacagacaggagatacctcacacatatccacaaaatttcccaatcaagaaagagggcaagtttgcctctttatccaaacttgtgcccagctctcctggaagcccctgtgctaagccagcgaggggcagtgagtgagcaaagcctggcggggtgtaaggaatcagggagctccctaggtctgtgtttgggtttagagaataaagacaagacccagaatgaatctaaccatctgtctcctagactggaatggggtccccagagccctgctcctgtcacagctgcccttaatcagttccccatgctgcaggcatgcagtgatataaataagtctaacctaggtccttcctttctcacagcctctatcaggaaccctcagctctaccccttgaagccctgtaaaggcactgcttccatgaccctgggctgcctggtaaaggactacttccctggtcctgtgactgtgacctggtattcagactccctgaacatgagcactgtgaacttccctgcccttggttctgaactcaaggtcaccaccagccaagtgaccagctggggcaagtcagccaagaacttcacatgccacgtgacacatcctccatcattcaacgaaagtaggactatcctaggtaagtagggatgggctgacagttacactgtgtattctcccttggagatggaacagtttctgtctaatcaggaacttgtcacaatttcctttcatagaggacttcataagagatttttttttctacttctatcatgtttagtgctccaaatagatttttaaaactggttgagtgcatattacttttagcctcagaagacatcatgtatatttaagaggcatttaactattgtaaattattctgatgactttaaaaaaagttaatgctgagttgtatatttttaaataaattttattagtttagtttaaaaaaagaaaagaaaattattaattttatttaaaaatctcctatatttaaaaaaaaaagagaaaaaagcagagctgggctggctacagttaccacaagaacatggtcagaggaggaagggactcttatacatacctatgacaggagaacgggagacccaacatactcgggggcctaccttcagagaacacaaggccagggcaatactcacagctcattgttcgaccctgccctagttcgacctgtcaacatcactgagcccaccttggagctactccattcatcctgcgaccccaatgctttccactccaccatccagctgtactgcttcatttatggccacatcctaaatgatgtctctgtcagctggcagatct

SEQ ID NO: 20 - DNA, 348 bp, Mus musculus
taatggacgatcgggagataactgatacacttgcacaaactgttctaatcaaggaggaaggcaaactagcctctacctgcagtaaactcaacatcactgagcagcaatggatgtctgaaagcaccttcacctgcaaggtcacctcccaaggcgtagactatttggcccacactcggagatgcccaggtaggtctacactcgcctgatgtccagacctcagagtcctgagggaaaggcaggctctcacacagcccttcctccccgacagatcatgagccacggggtgtgattacctacctgatcccacccagccccctggacctgtatcaaaacggtgctcccaagctt